SEQUENCE LISTING (3 sequences)

SEQ ID NO: 1 - protein, 300 aa, Homo sapiens
  Human CD38
MANCEFSPVSGDKPCCRLSRRAQLCLGVSILVLILVVVLAVVVPRWRQQWSGPGTTKRFPETVLARCVKYTEIHPEMRHVDCQSVWDAFKGAFISKHPCNITEEDYQPLMKLGTQTVPCNKILLWSRIKDLAHQFTQVQRDMFTLEDTLLGYLADDLTWCGEFNTSKINYQSCPDWRKDCSNNPVSVFWKTVSRRFAEAACDVVHVMLNGSRSKIFDKNSTFGSVEVHNLQPEKVQTLEAWVIHGGREDSRDLCQDPTIKELESIISKRNIQFSCKNIYRPDKFLQCVKNPEDSSCTSEI

SEQ ID NO: 2 - protein, 888 aa, Homo sapiens
  Human NAADP receptor Two Pore Channels TPC1
MESCYIAQAGLELLGSSSSPTLTSQSAEITEDASNGGVSEQHPWPSGFERELKPETISSPGYHILRATGEENMAVSLDDDVPLILTLDEGGSAPLAPSNGLGQEELPSKNGGSYAIHDSQAPSLSSGGESSPSSPAHNWEMNYQEAAIYLQEGENNDKFFTHPKDAKALAAYLFAHNHLFYLMELATALLLLLLSLCEAPAVPALRLGIYVHATLELFALMVVVFELCMKLRWLGLHTFIRHKRTMVKTSVLVVQFVEAIVVLVRQMSHVRVTRALRCIFLVDCRYCGGVRRNLRQIFQSLPPFMDILLLLLFFMIIFAILGFYLFSPNPSDPYFSTLENSIVSLFVLLTTANFPDVMMPSYSRNPWSCVFFIVYLSIELYFIMNLLLAVVFDTFNDIEKRKFKSLLLHKRTAIQHAYRLLISQRRPAGISYRQFEGLMRFYKPRMSARERYLTFKALNQNNTPLLSLKDFYDIYEVAALKWKAKKNREHWFDELPRTALLIFKGINILVKSKAFQYFMYLVVAVNGVWILVETFMLKGGNFFSKHVPWSYLVFLTIYGVELFLKVAGLGPVEYLSSGWNLFDFSVTVFAFLGLLALALNMEPFYFIVVLRPLQLLRLFKLKERYRNVLDTMFELLPRMASLGLTLLIFYYSFAIVGMEFFCGIVFPNCCNTSTVADAYRWRNHTVGNRTVVEEGYYYLNNFDNILNSFVTLFELTVVNNWYIIMEGVTSQTSHWSRLYFMTFYIVTMVVMTIIVAFILEAFVFRMNYSRKNQDSEVDGGITLEKEISKEELVAVLELYREARGASSDVTRLLETLSQMERYQQHSMVFLGRRSRTKSDLSLKMYQEEIQEWYEEHAREQEQQRQLSSSAAPAAQQPPGSRQRSQTVT

SEQ ID NO: 3 - protein, 752 aa, Homo sapiens
  Human NAADP receptor Two Pore Channels TPC2
MAEPQAESEPLLGGARGGGGDWPAGLTTYRSIQVGPGAAARWDLCIDQAVVFIEDAIQYRSINHRVDASSMWLYRRYYSNVCQRTLSFTIFLILFLAFIETPSSLTSTADVRYRAAPWEPPCGLTESVEVLCLLVFAADLSVKGYLFGWAHFQKNLWLLGYLVVLVVSLVDWTVSLSLVCHEPLRIRRLLRPFFLLQNSSMMKKTLKCIRWSLPEMASVGLLLAIHLCLFTMFGMLLFAGGKQDDGQDRERLTYFQNLPESLTSLLVLLTTANNPDVMIPAYSKNRAYAIFFIVFTVIGSLFLMNLLTAIIYSQFRGYLMKSLQTSLFRRRLGTRAAFEVLSSMVGEGGAFPQAVGVKPQNLLQVLQKVQLDSSHKQAMMEKVRSYGSVLLSAEEFQKLFNELDRSVVKEHPPRPEYQSPFLQSAQFLFGHYYFDYLGNLIALANLVSICVFLVLDADVLPAERDDFILGILNCVFIVYYLLEMLLKVFALGLRGYLSYPSNVFDGLLTVVLLVLEISTLAVYRLPHPGWRPEMVGLLSLWDMTRMLNMLIVFRFLRIIPSMKLMAVVASTVLGLVQNMRAFGGILVVVYYVFAIIGINLFRGVIVALPGNSSLAPANGSAPCGSFEQLEYWANNFDDFAAALVTLWNLMVVNNWQVFLDAYRRYSGPWSKIYFVLWWLVSSVIWVNLFLALILENFLHKWDPRSHLQPLAGTPEATYQMTVELLFRDILEEPGEDELTERLSQHPHLWLCR

(19) United States
(12) Patent Application Publication
(10) Pub. No.: US 2021/0087290 A1
(43) Pub. Date: Mar. 25, 2021

(54) COMPOUNDS WHICH SPECIFICALLY BIND TO CD38 FOR USE IN THE TREATMENT OF NEURODEGENERATIVE AND INFLAMMATORY DISEASES

(71) Applicant: ENCEFA, Saint-Maur-des-Fossés (FR)

(72) Inventors: Damien TOULORGE, Paris (FR); Serge GUERREIRO DA SILVA, Paris (FR); Laurence BRESSAC, Saint-Maur-des-Fossés (FR)

(73) Assignee: ENCEFA, Saint-Maur-des-Fossés (FR)

(21) Appl. No.: 16/633,749

(22) PCT Filed: Jul. 24, 2018
(86) PCT No.: PCT/EP2018/070064

§ 371 Date: Jan. 24, 2020

(30) Foreign Application Priority Data
Jul. 24, 2017 (EP) 17315006.1

(51) Int. Cl. C07K 16/28; A61K 39/395; A61P 25/00; A61P 39/00; A61P 29/00; A61K 45/06

(52) U.S. Cl. CPC C07K 16/2896; A61K 39/39541; A61P 25/00; A61P 39/00; A61K 2039/505; A61K 45/06; C07K 2317/92; C07K 2317/24; A61P 29/00

(57) ABSTRACT

A compound, which specifically binds to CD38, for use as a medicament in the prevention and/or treatment of a neurodegenerative disease and/or an inflammatory disease, by the opening of NAADP receptors Two Pore Channels TPC1 and/or TPC2, wherein the compound activates the opening of NAADP receptors Two Pore Channels TPC1 and/or TPC2.

### FIELD OF THE INVENTION

The present invention relates to the field of prevention and/or treatment of neurodegenerative diseases and/or of inflammatory diseases.

In particular, the invention relates to a compound, useful for the prevention and/or treatment of neurodegenerative and/or inflammatory diseases, said compound specifically binding to CD38 and activating the opening of NAADP receptors Two Pore Channels TPC1 and/or TPC2.

### BACKGROUND

Neurodegenerative disease is an umbrella term for a range of conditions which primarily affect the neurons in the human brain and spinal cord. Neurons are the building blocks of the nervous system, which includes the brain and spinal cord. Neurons normally don't reproduce or replace themselves, so when they become damaged or die, they cannot be replaced by the body. Examples of neurodegenerative diseases include Parkinson's disease, Alzheimer's disease or amyotrophic lateral sclerosis, which result in progressive degeneration and/or death of nerve cells. Symptoms of such diseases relate to impairments of movement (called ataxias) or mental functioning (called dementias).

The process of neurodegeneration is not well-understood so the diseases that stem from it have, as yet, no cures. Medications, brain surgery and multidisciplinary management are the therapeutic strategies used to provide relief from the symptoms in some patients. In the case of Parkinson's disease, levodopa has been the most widely used drug to reduce motor symptoms, however, its effect is temporary and invalidating side-effects occur with time. In Alzheimer's disease, drugs such as acetylcholine esterase inhibitors have also positive effects on the patients' symptoms but their efficacy is mainly observed at an early stage of the disease.

Strategies targeting the inhibition of degeneration of neurons are now explored. Agents currently under investigation include in particular calcium channel blockers (isradipine) or antagonists of the NAADP receptor, such as those described in WO2016024246. However, none of these agents are clinically validated.

Besides, known agents target only one type of neurodegenerative mechanism (e.g., excitotoxicity, oxidative stress or energy deficit due to mitochondrial abnormalities, etc.).

Consequently, there remains a significant need in the art for new and improved treatment for all type of patients affected by a neurodegenerative disorder, using agents able to target different types of neurodegenerative mechanisms.

CD38 is a 45 kDa type II transmembrane glycoprotein with a long C-terminal extracellular domain and a short N-terminal cytoplasmic domain CD38 have both a receptor-mediated function and an enzyme-mediated function. As a receptor, CD38 can interact with its ligand CD31. As an ectoenzyme, CD38 is a multi-functional protein that catalyzes several reactions, through:
(i) an NAD⁺ nucleosidase (NADase) activity, converting nicotinamide adenine dinucleotide (NAD⁺) into adenosine diphosphate-ribose (ADPR);
(ii) an ADP-ribosyl cyclase activity, converting NAD⁺ into cyclic ADPR (cADPR);
(iii) a cyclic ADP-ribose hydrolase activity, hydrolyzing cADPR into ADPR;
(iv) an NAADP synthase activity, converting, in the presence of nicotinic acid, NADP⁺ (the phosphorylated equivalent of NAD⁺) into nicotinic acid adenine dinucleotide phosphate (NAADP); and
(v) an NAADP hydrolase activity, converting NAADP into ADPR phosphate (ADPRP) (Malavasi et al., 2008. Physiol Rev. 88(3):841-886).

CD38 has been extensively studied in cancer research since this protein is upregulated in many hematopoietic malignancies and its expression is correlated with disease progression. Several antibodies were developed and used first as a research tool (clone 90, AT1, AT13/5, AT2, HB-7, IB4, IB6, OKT-10, SUN-4B7, T16) and then as therapeutics against cancer (including SAR650984 currently under phase III clinical trial [NCT02990338], MOR202 currently under phase II [NCT01421186]and Ab79 under preclinical development). In November 2015 and May 2016, the U.S. Food and Drug Administration and the European Medicines Agency, respectively, approved daratumumab for the treatment of multiple myeloma patients who previously received three or more prior lines of therapy.

Since CD38 is the main cellular NADase (Chini, 2009. Curr Pharm Des. 15(1):57-63), and that NAD⁺ or its precursors were shown to be neuroprotective in a large number of studies (Harlan et al., 2016. J Biol Chem. 291(20):10836-46; Lu et al., 2014. Exp Ther Mad. 8(3):943-50; Gong et al., 2013. Neurobiol Aging. 34(6):1581-8; US patent application US20120328526), CD38 was proposed as a new therapeutic target for the treatment of acute or chronic neurodegenerative diseases (Kristian et al., 2011. J Neurosci Res. 89(12):1946-55). Indeed, several studies using CD38 knock-out (KO) mice demonstrated that these animals showed significant protection against ischemic brain damage (Long et al., 2017. Neurochem Res. 42(1):283-93; US patent application US20120328526) as well as reduced Aβ production and neuroinflammation when crossed with Alzheimer's disease experimental mouse model APPswePS1ΔE9 (Blacher et al., 2015. Ann Neurol. 78(1):88-103) due to increased NAD⁺ levels, suggesting that inhibition of CD38 enzymatic activity might be neuroprotective by increasing NAD⁺ levels.

Unexpectedly, the Inventors have demonstrated herein that compounds, which specifically bind to CD38 and activate the opening of NAADP receptors Two Pore Channels TPC1 and/or TPC2, are able to efficiently prevent and protect neurons from degeneration by increasing cytosolic calcium levels.

These results are surprising since, contrary to what has been suggested in the state of the art, compounds which specifically bind to CD38 are demonstrated to be neuroprotective, not by increasing NAD⁺ levels as suggested in the literature, but by activating NAADP receptors TPCs, which are only very weakly activated following NAD⁺ treatment. This is in total contradiction with previously cited studies using CD38 KO mice (Long et al., 2017. Neurochem Res. 42(1):283-93; Blacher et al., 2015. Ann Neurol. 78(1):88-103; US patent application US20120328526); with a publication of Hockey et al. (2015. J Cell Sci. 128(2):232-8) who showed that lysosomal abnormalities in fibroblasts from Parkinson's disease patients (i) were corrected by NAADP receptors' inhibition and (ii) were exaggerated by NAADP; as well as with a study published by Fernandez et al. (2016. Autophagy. 12(9):1487-506) who showed that the inhibition of NAADP receptors reduces cytoplasmic iron levels, which are elevated in various neurodegenerative diseases.

This is also in total contradiction with current investigations aiming to develop agents able to inhibit the degeneration of neurons by blocking calcium channel (e.g., isradipine) or by antagonizing NAADP receptors (e.g., such as those described in WO2016024246), thus reducing cytoplasmic calcium levels.

CD38 is strongly expressed in peripheral blood mononuclear cells (PBMCs) (Malavasi et al., 2008. Physiological Review. 88:841-886) including B cells, T cells, monocytes and NK cells (Krejcik et al., 2016. Blood. 128(3):384-94) and its expression was shown to be induced by inflammatory cytokines, endotoxins and interferon (for review see Chini et al., 2018. Trends Pharmacol Sci. 39(4):424-36). More generally, increased CD38 expression is considered as a marker of pro-inflammatory M1 state of activation (Jablonski et al., 2015. PLOS One. 10(12):e0145342). In line with these evidences, increased CD38 expression was observed in a rat model of experimental autoimmune encephalomyelitis (Herrmann et al., 2016. Dis Model Mech. 9(10):1211-20), in human T cells from systemic lupus erythematosus patients (Pavon et al., 2006. Mol Immunol. 43(7):1029-39) as well as in rheumatoid arthritis synovial tissues (Chang et al., 2014. Clin Exp Immunol. 176(2):222-31).

Targeting CD38 with anti-CD38 antibodies daratumumab or isatuximab was found to decrease plasma levels of anti-inflammatory interleukin-10 (IL-10) (Krejcik et al., 2016. Blood. 128(3):384-94; Feng et al., 2017. Clin Cancer Res. 23(15):4290-4300). Unexpectedly, and in contradiction with these results, we found that compounds, which specifically bind to CD38 and activate the opening of NAADP receptors Two Pore Channels TPC1 and/or TPC2, strongly increased plasma IL-10 levels in vivo.

### SUMMARY

The present invention relates to a compound which specifically binds to CD38, for use as a medicament in the prevention and/or treatment of a neurodegenerative disease and/or an inflammatory disease, by the opening of NAADP receptors Two Pore Channels TPC1 and/or TPC2, wherein said compound activates the opening of NAADP receptors Two Pore Channels TPC1 and/or TPC2.

In one embodiment, the compound for use according to the invention is selected from the group comprising:
an antibody, an antigen-binding fragment thereof or an antigen-binding antibody mimetic; and
a small organic molecule.

In one embodiment, the compound for use according to the invention increases intracellular NAADP levels in neurons and/or immune cells, by inhibiting the NAADP hydrolase activity of CD38 or by activating the NAADP synthase activity of CD38.

In one embodiment, the compound for use according to the invention is an anti-CD38 antibody, an antigen binding fragment thereof or an antigen-binding antibody mimetic, which specifically binds to a peptide comprising amino acids 220 to 285 of SEQ ID NO: 1.

In one embodiment, the compound for use according to the invention is an anti-CD38 antibody, an antigen binding fragment thereof or an antigen-binding antibody mimetic, which specifically binds to cysteine 254 and/or cysteine 275 of SEQ ID NO: 1.

In one embodiment, the compound for use according to the invention is an anti-CD38 antibody or an antigen binding fragment thereof or an antigen-binding antibody mimetic, which specifically binds to the 5^{th} C-terminal disulfide loop involving cysteine 254 and cysteine 275 of SEQ ID NO: 1.

In one embodiment, the compound for use according to the invention induces CD38 internalization.

In one embodiment, the compound for use according to the invention is an anti-CD38 antibody, an antigen binding fragment thereof or an antigen-binding antibody mimetic, which specifically binds to human CD38 with a K_{D} inferior or equal to 10⁻⁷, preferably as may be determined by biosensor analysis.

In one embodiment, the compound for use according to the invention is a humanized monoclonal antibody.

In one embodiment, the compound for use according to the invention is a small organic molecule which inhibits the NAADP hydrolase activity of CD38 with an IC₅₀ inferior or equal to 5 μM or activates the NAADP synthase activity of CD38 with an EC₅₀ inferior or equal to 5 μM.

In one embodiment, the compound for use according to the invention is a small organic molecule, which specifically binds to at least one amino acid of human CD38 with SEQ ID NO: 1 selected from the group comprising glutamic acid 146, aspartic acid 155 and glutamic acid 226.

In one embodiment, the said neurodegenerative disease is selected from the group comprising Parkinson's disease and related disorders including Parkinson's disease, Parkinson-dementia, autosomal recessive PARK2 and PARK6-linked Parkinsonism, atypical parkinsonian syndromes, including, progressive supranuclear palsy, corticobasal degeneration syndrome, Lewy bodies dementia, multiple system atrophy, Guadeloupean Parkinsonism and Lytigo-bodig disease; motor neuron diseases including amyotrophic lateral sclerosis, frontotemporal dementia, progressive bulbar palsy, pseudobulbar palsy, primary lateral sclerosis, progressive muscular atrophy, spinal muscular atrophy and post-polio syndrome; neuro-inflammatory diseases; Alzheimer's disease and related disorders including early stage of an Alzheimer's disorder, mild stage of an Alzheimer's disorder, moderate stage of an Alzheimer's disorder, mild to moderate stage of an Alzheimer's disorder, advanced stage of an Alzheimer's disorder, mild cognitive impairment, vascular dementia, mixed dementia, Pick's disease, argyrophilic grain disease, posterior cortical atrophy, Wernicke-Korsakoff Syndrome; prion diseases; lysosomal storage diseases; leukodystrophies; Huntington's Disease; multiple sclerosis; Down syndrome; spinal and bulbar muscular atrophy; HIV-Associated Neurocognitive Disorder; Tourette Syndrome; autosomal dominant spinocerebellar ataxia; Friedreich's Ataxia; Dentatorubral pallidoluysian atrophy; myotonic dystrophy; schizophrenia; age associated memory impairment; autism and autism spectrum disorders; attention-deficit hyperactivity disorder; chronic pain; alcohol-induced dementia; progressive non-fluent aphasia; semantic dementia; spastic paraplegia; fibromyalgia; post-Lyme disease; neuropathies; withdrawal symptoms; Alpers' disease; cerebro-oculo-facio-skeletal syndrome; Wilson's disease; Cockayne syndrome; Leigh's disease; neurodegeneration with brain iron accumulation; opsoclonus myoclonus syndrome; alpha-methylacyl-CoA racemase deficiency; Andermann syndrome; Arts syndrome; Marinesco-Sjögren syndrome; mitochondrial membrane protein-associated neurodegeneration; pantothenate kinase-associated neurodegeneration; polycystic lipomembranous osteodysplasia with sclerosing leukoencephalopathy; riboflavin transporter deficiency neuronopathy; and ataxia telangiectasia.

In one embodiment, the said neurodegenerative disease is selected from the group comprising Parkinson's disease, Lewy body dementia, multiple system atrophy, Alzheimer's disease, progressive supranuclear palsy, corticobasal degeneration syndrome, frontotemporal dementia, amyotrophic lateral sclerosis, spinal and bulbar muscular atrophy, stroke, traumatic brain injuries, Huntington's disease, multiple sclerosis, Friedreich's ataxia, Charcot-Marie-Tooth disease, Creutzfeld-Jacob disease and other prion diseases, leukodistrophies, lysosomal storage disorders.

In one embodiment, the said inflammatory disease is selected from the group comprising neuroinflammatory diseases, Gaucher's disease, autoimmune diseases, allergy, asthma, hepatitis, reperfusion injury, type 2 diabetes and transplant rejection.

The present invention also relates to combination product comprising as active ingredients:
at least one compound according to the invention; and
at least one second therapeutic agent selected from the group comprising neuroprotective agents, symptomatic agents, probiotics and antibodies used to neutralize aggregated or aggregation-prone proteins;
for use as a medicament for the prevention and/or treatment of a neurodegenerative disease and/or inflammatory disease, by the opening of NAADP receptors Two Pore Channels TPC1 and/or TPC2, wherein said active ingredients are formulated for separate, simultaneous, or sequential administration.

The present invention also relates to a method of manufacturing a compound according to the invention, which comprises the step of selecting a compound which specifically binds to SEQ ID NO: 1 and which activates the opening of NAADP receptors Two Pore Channels TPC1 and/or TPC2.

In one embodiment, the method of the invention further comprises a step of selecting a compound which inhibits the NAADP hydrolase activity of CD38 or which activates the NAADP synthase activity of CD38.

### DETAILED DESCRIPTION OF THE INVENTION

In one aspect, the present invention relates to a compound, which specifically binds to CD38, particularly to human CD38, for use as a medicament in the prevention and/or treatment of a neurodegenerative disease and/or inflammatory disease, by the opening of NAADP receptors Two Pore Channels TPC1 and/or TPC2, wherein said compound activates the opening of NAADP receptors Two Pore Channels TPC1 and/or TPC2.

The use of a compound, which specifically binds to CD38 and activates the opening of NAADP receptors Two Pore Channels TPC1 and/or TPC2, as a medicament has in particular the following advantages:
it efficiently prevents directly and indirectly different types of neurons from different types of degeneration:
   the neuroprotection is not specific to a particular type of neuron. On the contrary, it is able to efficiently protect in vitro different types of neurons including cortical neurons comprising glutamatergic and GABAergic cells, as well as dopaminergic neurons from midbrain cultures;
   it is able to directly protect neurons from different types of degeneration mechanisms including excitotoxicity, oxidative stress, energy deficit due to mitochondrial complex I inhibition, electrical activity deficit, as well as neurotrophic factor deprivation;
   it is able to indirectly protect neurons from degeneration, having an anti-inflammatory effect by limiting the number of microglial cells and astrocytes.
it is useful for all type of patients affected by a neurodegenerative disorder considering that neurodegenerative disorders involve different types of neurons and neurodegenerative mechanisms;
it has reduced side effects due to the specificity of compound;
it is efficient at every stage of neurodegenerative diseases;
its effect is not temporary;
it is highly efficient using low doses compared to the other tested agents (e.g., HB7 antibody totally prevented dopaminergic neurons cell death at a concentration of 7.5 nM, whereas NAD⁺ exerted its neuroprotective effect at a 3 mM concentration);
it has anti-inflammatory properties by increasing plasma interleukin-10 levels.

Unless defined otherwise, all technical and scientific terms used herein have the same meaning as commonly understood to one skilled in the relevant art.

For convenience, the meaning of certain terms and phrases employed in the specification, examples and claims are provided.

As used herein, the term “compound, which specifically binds to CD38” relates to any molecule suitable for pharmaceutical uses, which specifically binds to CD38. It encompasses antibodies, antigen-binding fragments thereof, antigen-binding antibody mimetics, small organic molecules, oligonucleotides and recombinant proteins.

In an embodiment, the compound, which specifically binds to CD38, according to the invention is selected from the group comprising or consisting of:
an antibody, an antigen-binding fragment thereof, an antigen-binding antibody mimetic; and
a small organic molecule.

As used herein, the term “antibody” comprises polyclonal antibodies, monoclonal antibodies, recombinant, bispecific, multispecific or modified antibodies.

As used herein, a “monoclonal antibody” is intended to refer to a preparation of antibody molecules, antibodies which share a common heavy chain and common light chain amino acid sequence, in contrast with “polyclonal antibody” preparations which contain a mixture of antibodies of different amino acid sequence. Monoclonal antibodies can be generated by several known technologies like phage, bacteria, yeast or ribosomal display, as well as by classical methods exemplified by hybridoma-derived antibodies. Thus, the term “monoclonal” is used to refer to all antibodies derived from one nucleic acid clone.

The antibodies of the present invention include recombinant antibodies. As used herein, the term “recombinant antibody” refers to antibodies which are produced, expressed, generated or isolated by recombinant means, such as antibodies which are expressed using a recombinant expression vector transfected into a host cell; antibodies isolated from a recombinant combinatorial antibody library; antibodies isolated from an animal (e.g., a mouse) which is transgenic due to human immunoglobulin genes; or antibodies which are produced, expressed, generated or isolated in any other way in which particular immunoglobulin gene sequences (such as human immunoglobulin gene sequences) are assembled with other DNA sequences. Recombinant antibodies include, for example, chimeric and humanized antibodies.

As used herein, a “chimeric antibody” refers to an antibody in which the sequence of the variable domain derived from the germline of a mammalian species, such as a mouse, have been grafted onto the sequence of the constant domain derived from the germline of another mammalian species, such as a human.

As used herein, a “humanized antibody” refers to an antibody in which CDR sequences derived from the germline of another mammalian species, such as a mouse, have been grafted onto human framework sequences.

In an embodiment, the invention relates to an anti-CD38 antibody or antigen-binding fragment thereof as defined above that is a humanized monoclonal antibody.

As used herein, an “antigen-binding fragment of an antibody” means a part of an antibody, i.e., a molecule corresponding to a portion of the structure of the antibody of the invention, that exhibits antigen-binding capacity for CD38, possibly in its native form; such fragment especially exhibits the same or substantially the same antigen-binding specificity for said antigen compared to the antigen-binding specificity of the corresponding four-chain antibody. Advantageously, the antigen-binding fragments have a similar binding affinity as the corresponding 4-chain antibodies. However, antigen-binding fragments that have a reduced antigen-binding affinity with respect to corresponding 4-chain antibodies are also encompassed within the invention. The antigen-binding capacity can be determined by measuring the affinity between the antibody and the target fragment. These antigen-binding fragments may also be designated as “functional fragments” of antibodies.

Antigen-binding fragments of antibodies are fragments which comprise their hypervariable domains designated CDRs (Complementary Determining Regions) or part(s) thereof encompassing the recognition site for the antigen, i.e., CD38, thereby defining antigen recognition specificity.

Each light and heavy chain variable domains (respectively VL and VH) of a four-chain immunoglobulin has three CDRs, designated VL-CDR1 (or LCDR1), VL-CDR2 (or LCDR2), VL-CDR3 (or LCDR3) and VH-CDR1 (or HCDR1), VH-CDR2 (or HCDR2), VH-CDR3 (or HCDR3), respectively.

The skilled person is able to determine the location of the various regions/domains of antibodies by reference to the standard definitions in this respect set forth, including a reference numbering system, a reference to the numbering system of KABAT or by application of the IMGT “collier de perle” algorithm. In this respect, for the definition of the sequences of the invention, it is noted that the delimitation of the regions/domains may vary from one reference system to another. Accordingly, the regions/domains as defined in the present invention encompass sequences showing variations in length or localization of the concerned sequences within the full-length sequence of the variable domains of the antibodies, of approximately +/−10%.

Based on the structure of four-chain immunoglobulins, antigen-binding fragments can thus be defined by comparison with sequences of antibodies in the available databases and prior art, and especially by comparison of the location of the functional domains in these sequences, noting that the positions of the framework and constant domains are well defined for various classes of antibodies, especially for IgGs, in particular for mammalian IgGs. Such comparison also involves data relating to 3-dimensional structures of antibodies.

For illustration purpose of specific embodiments of the invention, antigen binding fragments of an antibody that contain the variable domains comprising the CDRs of said antibody encompass Fv, dsFv, scFv, Fab, Fab′, F(ab′)2, and single-domain antibody. Fv fragments consist of the VL and VH domains of an antibody associated together by hydrophobic interactions; in dsFv fragments, the VH:VL heterodimer is stabilised by a disulfide bond; in scFv fragments, the VL and VH domains are connected to one another via a flexible peptide linker thus forming a single-chain protein. Fab fragments are monomeric fragments obtainable by papain digestion of an antibody; they comprise the entire L chain, and a VH-CH1 fragment of the H chain, bound together through a disulfide bond. The F(ab′)2 fragment can be produced by pepsin digestion of an antibody below the hinge disulfide; it comprises two Fab′ fragments, and additionally a portion of the hinge region of the immunoglobulin molecule. The Fab′ fragments are obtainable from F(ab′)2 fragments by cutting a disulfide bond in the hinge region. F(ab′)2 fragments are divalent, i.e., they comprise two antigen binding sites, like the native immunoglobulin molecule; on the other hand, Fv (a VHVL dimmer constituting the variable part of Fab), dsFv, scFv, Fab, and Fab′ fragments are monovalent, i.e., they comprise a single antigen-binding site. These basic antigen-binding fragments of the invention can be combined together to obtain multivalent antigen-binding fragments, such as diabodies, tribodies or tetrabodies. These multivalent antigen-binding fragments are also part of the present invention.

As used herein, the term “bispecific antibodies” refers to antibodies that recognize two different antigens by virtue of possessing at least one region (e.g., derived from a variable region of a first antibody) that is specific for a first antigen, and at least a second region (e.g., derived from a variable region of a second antibody) that is specific for a second antigen. A bispecific antibody specifically binds to two target antigens and is thus one type of multispecific antibody. Multispecific antibodies, which recognize two or more different antigens, can be produced by recombinant DNA methods or include, but are not limited to, antibodies produced chemically by any convenient method. Bispecific antibodies include all antibodies or conjugates of antibodies, or polymeric forms of antibodies which are capable of recognizing two different antigens. Bispecific antibodies include antibodies that have been reduced and reformed so as to retain their bivalent characteristics and to antibodies that have been chemically coupled so that they can have several antigen recognition sites for each antigen such as BiME (Bispecific Macrophage Enhancing antibodies), BiTE (bispecific T cell engager), DART (Dual affinity retargeting); DNL (dock-and-lock), DVD-Ig (dual variable domain immunoglobulins), HAS (human serum albumin), kih (knobs into holes).

Accordingly, bispecific antibodies of the invention specifically bind to CD38 and a second antigen and activate the opening of NAADP receptors Two Pore Channels TPC1 and/or TPC2.

In an embodiment, the invention also encompasses anti-CD38 antibodies or antigen-binding fragments thereof or antigen-binding antibody mimetics as defined herein, that are bispecific, in particular for their use as a medicament in the prevention and/or treatment of a neurodegenerative and/or inflammatory disease, by the opening of NAADP receptors Two Pore Channels TPC1 and/or TPC2.

Several researches to develop therapeutic antibodies had led to engineer the Fc regions to optimize antibody properties allowing the generation of molecules that are better suited to the pharmacology activity required of them. The Fc region of an antibody mediates its serum half-life and effector functions, such as complement-dependent cytotoxicity (CDC), antibody-dependent cellular cytotoxicity (ADCC) and antibody-dependent cell phagocytosis (ADCP). Several mutations located at the interface between the CH2 and CH3 domains, such as T250Q/M428L and M252Y/S254T/T256E+H433K/N434F, have been shown to increase the binding affinity to FcRn and the half-life of IgG1 in vivo. However, there is not always a direct relationship between increased FcRn binding and improved half-life. One approach to improve the efficacy of a therapeutic antibody is to increase its serum persistence, thereby allowing higher circulating levels, less frequent administration and reduced doses. Engineering Fc regions may be desired to either reduce or increase the effector function of the antibody. For antibodies that target cell-surface molecules, especially those on immune cells or neurons, abrogating effector functions is required. Conversely, for antibodies intended for oncology use, increasing effector functions may improve the therapeutic activity. The four human IgG isotypes bind the activating Fcγ receptors (FcγRI, FcγRIIa, FcγRIIIa), the inhibitory FcγRIIb receptor, and the first component of complement (Clq) with different affinities, yielding very different effector functions. Binding of IgG to the FcγRs or C1q depends on residues located in the hinge region and the CH2 domain. Two regions of the CH2 domain are critical for FcγRs and C1q binding, and have unique sequences in IgG2 and IgG4.

As used herein, a “modified antibody” corresponds to a molecule comprising an antibody or an antigen-binding fragment thereof, wherein said antibody or fragment thereof is associated with a functionally different molecule. A modified antibody of the invention may be either a fusion chimeric protein or a conjugate resulting from any suitable form of attachment including covalent attachment, grafting, chemical bonding with a chemical or biological group or with a molecule, such as a PEG polymer or another protective group or molecule suitable for protection against proteases cleavage in vivo, for improvement of stability and/or half-life of the antibody or functional fragment. With similar techniques, especially by chemical coupling or grafting, a modified antibody can be prepared with a biologically active molecule, said active molecule being for example chosen among toxins, in particular Pseudomonas exotoxin A, the A-chain of plant toxin ricin or saporin toxin, especially a therapeutic active ingredient, a vector (including especially a protein vector) suitable for targeting the antibody or functional fragment to specific cells or tissues of the human body, or it may be associated with a label or with a linker, especially when fragments of the antibody are used. PEGylation of the antibody or functional fragments thereof is a particular interesting embodiment as it improves the delivery conditions of the active substance to the host, especially for a therapeutic application. PEGylation can be site specific to prevent interference with the recognition sites of the antibodies or functional fragments, and can be performed with high molecular weight PEG. PEGylation can be achieved through free cysteine residues present in the sequence of the antibody or functional fragment or through added free Cysteine residues in the amino sequence of the antibody or functional fragment.

In an embodiment, the invention also encompasses anti-CD38 antibodies or antigen-binding fragments thereof or antigen-binding antibody mimetics as defined herein that are modified, in particular for their use as a medicament in the prevention and/or treatment of a neurodegenerative and/or inflammatory disease, by the opening of NAADP receptors Two Pore Channels TPC1 and/or TPC2.

As used herein the term “antigen-binding antibody mimetic” refers to artificial proteins, peptides and any chemical compounds with the capacity to bind antigens mimicking that of antibodies.

Such mimetics comprise oligonucleotide aptamers. As used herein, the term “oligonucleotide” relates to a short molecule of nucleotides including DNA, RNA but also modified nucleotides (such as nucleotides comprising at least one chemical modification). In particular, said oligonucleotide consists of less than 200 nucleotides, more particularly of less than 50 nucleotides. As used herein, the term “oligonucleotide aptamer” refers to short oligonucleotides that can selectively bind to small molecular ligands or protein targets with high affinity and specificity, when folded into their unique three-dimensional structures.

Such mimetics comprise affitins and anticalins as well as peptide aptamers. Affitins are artificial proteins with the ability to selectively bind antigens. They are structurally derived from the DNA binding protein Sac7d, found in Sulfolobus acidocaldarius, a microorganism belonging to the archaeal domain. By randomizing the amino acids on the binding surface of Sac7d, e.g., by generating variants corresponding to random substitutions of 11 residues of the binding interface of Sac7d, an affitin library may be generated and subjecting the resulting protein library to rounds of ribosome display, the affinity can be directed towards various targets, such as peptides, proteins, viruses and bacteria. Affitins are antibody mimetics and are being developed as tools in biotechnology. They have also been used as specific inhibitors for various enzymes (Krehenbrink et al., 2008. J Mol Biol. 383(5):1058-68). The skilled person may readily develop anticalins with the required binding properties using methods know in the art, in particular as disclosed in International patent application WO2008068637 and the above-cited publication, in particular the generation of phage display and/or ribosome display libraries and their screening using an antigen as dislosed herein. Anticalins are artificial proteins that are able to bind to antigens, either to proteins or to small molecules. They are antibody mimetic derived from human lipocalins which are a family of naturally binding proteins. Anticalins are about eight times smaller with a size of about 180 amino acids and a mass of about 20 kDa (Kolmar & Skerra, 2008. FEBS J. 275(11):2667). Anticalin phage display libraries have been generated which allow for the screening and selection, in particular of anticalins with specific binding properties. The skilled person may readily develop affitins with the required binding properties using methods known in the art, in particular as disclosed in EP patent EP1270725, US patent U.S. Pat. No. 8,536,307, Schlehuber & Skerra (2002. Biophys Chem. 96(2-3):213-28) and the above-cited publication, in particular the generation of phage display and/or ribosome display libraries and their screening using an antigen as disclosed herein. As used herein the term “peptide aptamers” refers to small combinatorial proteins that are selected to bind to specific sites on their target molecules (antigens). Anticalins and affitins and peptide aptamers may be produced in a number of expression system comprising bacterial expression systems or by combinatorial chemistry. Thus, the invention provides affitins, anticalins, peptide aptamers and other similar antibody mimetics with the features of the antibodies described herein, in particular with regard to the specific binding to CD38 and to the activation of the opening of NAADP receptors Two Pore Channels TPC1 and/or TPC2.

All the embodiments disclosed herein for antibodies or fragments thereof are transposed mutatis mutandis to the antigen-binding antibody mimetics of the invention.

As used herein, the term “CD38” refers to the 45 kDa type II transmembrane glycoprotein also known as T10, cyclic ADP-ribose hydrolase 1, ADPRC1 as described in Malavasi et al. (2008. Physiological Review. 88:841-886), particularly from a mammal species, more particularly a human CD38.

Preferably, the term “human CD38” refers to the protein of amino acid sequence SEQ ID NO: 1, referenced by the NP_001766 NCBI accession number. The numbering of amino acids of human CD38 as described herein corresponds to the numbering of amino acids of the human CD38 sequence set forth in SEQ ID NO: 1, and referenced by the NP_001766 NCBI accession number.

SEQ ID NO: 1
MANCEFSPVSGDKPCCRLSRRAQLCLGVSILVLILVVVLAVVVPRWRQQW
SGPGTTKRFPETVLARCVKYTEIHPEMRHVDCQSVWDAFKGAFISKHPCN
ITEEDYQPLMKLGTQTVPCNKILLWSRIKDLAHQFTQVQRDMFTLEDTLL
GYLADDLTWCGEFNTSKINYQSCPDWRKDCSNNPVSVFWKTVSRRFAEAA
CDVVHVMLNGSRSKIFDKNSTFGSVEVHNLQPEKVQTLEAWVIHGGREDS
RDLCQDPTIKELESIISKRNIQFSCKNIYRPDKFLQCVKNPEDSSCTSEI

As used herein, the term “anti-CD38 antibody” refers to an antibody which specifically binds to CD38, in particular to a human CD38.

The specific binding between the compound according to the invention (in particular the antibody or antigen-binding fragment thereof or antigen-binding antibody mimetic according to the invention) and CD38 (in particular the epitope within CD38) implies that the compound according to the invention (in particular, the antibody or antigen-binding fragment thereof or antigen-binding antibody mimetic according to the invention; the small organic molecule according to the invention; or the oligonucleotide according to the invention) exhibits appreciable affinity for CD38 (in particular the epitope within CD38).

When the compound is an antibody or antigen-binding fragment thereof or antigen-binding antibody mimetic, “appreciable affinity” includes binding with an affinity of about 10⁻⁷, preferably of about 10⁻⁸ M (K_{D}-affinity constant) or stronger. Preferably, binding is considered specific when the binding affinity ranges between about 10⁻⁷ M and about 10⁻¹² M, optionally between about 10⁻⁸ and about 10⁻¹², optionally between about 10⁻⁹M and about 10⁻¹¹M, in particular is of about 10⁻¹⁹ M.

When the compound is a small organic molecule, “appreciable affinity” includes binding with an affinity of about 10⁻⁶ M (K_{D}-affinity constant) or stronger. Preferably, binding is considered specific when the binding affinity ranges between about 10⁻⁶ M and about 10⁻¹⁰M, optionally between about 10⁻⁷M and about 10⁻⁹M, in particular is of about 10⁻⁸ M.

When the compound is an oligonucleotide, “appreciable affinity” includes binding with an affinity of about 200 nM (K_{D}-affinity constant) or stronger. Preferably, binding is considered specific when the binding affinity ranges between about 10 nM and about 200 nM, optionally between about 50 nM and about 150 nM, in particular is of about 100 nM.

Whether a binding domain specifically reacts with or binds to a target can be tested readily by, inter alia, comparing the reaction of said binding domain with a target protein or antigen with the reaction of said binding domain with proteins or antigens other than the target protein.

The affinity can be determined by various methods well-known from one skilled in the art. These methods include, but are not limited to, Biacore Analysis, Blitz analysis and Scatchard plot.

In an embodiment, the anti-CD38 antibody or antigen-binding fragment thereof or antigen-binding antibody mimetic according to the invention has a K_{D} value (affinity constant) inferior or equal to about 10⁻⁷M, preferably inferior or equal to about 10⁻⁸, more preferably inferior or equal to about 10⁻⁹ M for CD38, even more preferably inferior or equal to about 10⁻¹⁹ M, preferably as may be determined by biosensor analysis, particularly by Biacore Analysis.

As used herein, the term “NAADP receptors Two Pore Channels TPC1 and/or TPC2” relates to the receptors also known as TPCN1 and TPCN2 as described in Patel et al. (2015. Sci Signal. 8(384):re7).

Preferably, the term “NAADP receptor Two Pore Channels TPC1” refers to the human TPC1 of amino acid sequence SEQ ID NO: 2, referenced by the NP_001137291.1 NCBI accession number.

SEQ ID NO: 2
MESCYIAQAGLELLGSSSSPTLTSQSAEITEDASNGGVSEQHPWPSGFER
ELKPETISSPGYHILRATGEENMAVSLDDDVPLILTLDEGGSAPLAPSNG
LGQEELPSKNGGSYAIHDSQAPSLSSGGESSPSSPAHNWEMNYQEAAIYL
QEGENNDKFFTHPKDAKALAAYLFAHNHLFYLMELATALLLLLLSLCEAP
AVPALRLGIYVHATLELFALMVVVFELCMKLRWLGLHTFIRHKRTMVKTS
VLVVQFVEAIVVLVRQMSHVRVTRALRCIFLVDCRYCGGVRRNLRQIFQS
LPPFMDILLLLLFFMIIFAILGFYLFSPNPSDPYFSTLENSIVSLFVLLT
TANFPDVMMPSYSRNPWSCVFFIVYLSIELYFIMNLLLAVVFDTFNDIEK
RKFKSLLLHKRTAIQHAYRLLISQRRPAGISYRQFEGLMRFYKPRMSARE
RYLTFKALNQNNTPLLSLKDFYDIYEVAALKWKAKKNREHWPDELPRTAL
LIFKGINILVKSKAFQYFMYLVVAVNGVWILVETFMLKGGNPPSKHVPWS
YLVFLTIYGVELFLKVAGLGPVEYLSSGWNLFDFSVTVFAFLGLLALALN
MEPFYFIVVLRPLQLLRLFKLKERYRNVLDTMFELLPRMASLGLTLLIFY
YSFAIVGMEFFCGIVFPNCCNTSTVADAYRWRNHTVGNRTVVEEGYYYLN
NFDNILNSFVTLFELTVVNNWYIIMEGVTSQTSHWSRLYFMTFYIVTMVV
MTIIVAFILEAFVFRMNYSRKNQDSEVDGGITLEKEISKEELVAVLELYR
EARGASSDVTRLLETLSQMERYQQHSMVFLGRRSRTKSDLSLKMYQEEIQ
EWYEEHAREQEQQRQLSSSAAPAAQQPPGSRQRSQTVT

Preferably, the term “NAADP receptor Two Pore Channels TPC2” refers to the human TPC2 of amino acid sequence SEQ ID NO: 3, referenced by the NP_620714.2 NCBI accession number.

SEQ ID NO: 3
MAEPQAESEPLLGGARGGGGDWPAGLTTYRSIQVGPGAAARWDLCIDQAV
VFIEDAIQYRSINHRVDASSMWLYRRYYSNVCQRTLSFTIFLILFLAFIE
TPSSLTSTADVRYRAAPWEPPCGLTESVEVLCLLVFAADLSVKGYLFGWA
HFQKNLWLLGYLVVLVVSLVDWTVSLSLVCHEPLRIRRLLRPFFLLQNSS
MMKKTLKCIRWSLPEMASVGLLLAIHLCLFTMFGMLLFAGGKQDDGQDRE
RLTYFQNLPESLTSLLVLLTTANNPDVMIPAYSKNRAYAIFFIVFTVIGS
LFLMNLLTAIIYSQFRGYLMKSLQTSLFRRRLGTRAAFEVLSSMVGEGGA
FPQAVGVKPQNLLQVLQKVQLDSSHKQAMMEKVRSYGSVLLSAEEFQKLF
NELDRSVVKEHPPRPEYQSPFLQSAQFLFGHYYFDYLGNLIALANLVSIC
VFLVLDADVLPAERDDFILGILNCVFIVYYLLEMLLKVFALGLRGYLSYP
SNVFDGLLTVVLLVLEISTLAVYRLPHPGWRPEMVGLLSLWDMTRMLNML
IVFRFLRIIPSMKLMAVVASTVLGLVQNMRAFGGILVVVYYVFAIIGINL
FRGVIVALPGNSSLAPANGSAPCGSFEQLEYWANNFDDFAAALVTLWNLM
VVNNWQVFLDAYRRYSGPWSKIYFVLWWLVSSVIWVNLFLALILENFLHK
WDPRSHLQPLAGTPEATYQMTVELLFRDILEEPGEDELTERLSQHPHLWL
CR

As used herein, the term “activates the opening of NAADP receptors Two Pore Channels TPC1 and/or TPC2” relates to the activation of NAADP receptors Two Pore Channels TPC1 and/or TPC2 leading to their opening and the release of Ca²⁺ (as described in Patel et al., 2015. Sci Signal. 8(384):re7) from lysosomal Ca²⁺ stores to the cytoplasm.

The capacity of a compound to activate the opening of NAADP receptors Two Pore Channels TPC1 and/or TPC2 can be tested by using an inhibitor of TPC1 and/or TPC2 activation, such as NedK (Davidson et al., 2015. Cardiovasc Res. 108(3):357-66) or Ned-19 (Arndt et al., 2014. Mol Biol Cell. 25(6):948-64; Calcraft et al., 2009. Nature. 459(7246):596-600; Naylor et al., 2009. Nat Chem Biol. 5(4):220-6; Lee et al., 2016. Sci Rep. 6:20282), as will be further described in the Examples section below.

In particular, the neuroprotective effect of a compound according to the invention, which specifically binds to CD38, is antagonized by at least 20%, 30%, 40%, 50%, particularly at least 60% and more particularly at least 70% in the presence of NedK and/or Ned-19, as compared to a negative control in the absence of NedK and/or Ned-19 in a neuroprotective assay as will be further described in the Examples section below. These neuroprotective assays include, but are not limited to, assays on prevention of spontaneous and progressive dopaminergic (DA) neuron death in midbrain cultures, protection of dopaminergic neurons against the mitochondrial neurotoxin MPP⁺, protection of dopaminergic neurons from neurotrophic factor deprivation, protection of cortical neurons from oxidative stress, and protection of cortical neurons from excitotoxicity, in the presence or absence of Ned-19, as will be further described in the Examples section below.

In particular, the anti-inflammatory effect of a compound according to the invention, which specifically binds to CD38, is antagonized by at least 10%, 20%, 25%, 30%, particularly at least 35% and more particularly at least 40% in the presence of NedK and/or Ned-19, as compared to a negative control in the absence of NedK and/or Ned-19 in an inflammation assay as will be further described in the Examples section below. These inflammation assays include, but are not limited to, assays quantifying in vitro the increase in microglial cell number upon treatment with the mitochondrial neurotoxin MPP⁺, or assays quantifying in vivo the increase of microglial cell and/or astrocytes number upon treatment with Conduritol β epoxide (CBE), in the presence or absence of Ned-19, as will be further described in the Examples section below.

In one embodiment, the compound according to the invention, which specifically binds to CD38 and activates the opening of NAADP receptors Two Pore Channels TPC1 and/or TPC2 according to the invention, has the advantage to directly and/or indirectly protect different type of neurons from different types of neurodegenerative mechanisms.

The compound according to the invention, which specifically binds to CD38 and activates the opening of NAADP receptors Two Pore Channels TPC1 and/or TPC2 according to the present invention has at least one of the following properties, in particular the whole properties:
it prevents spontaneous and progressive cell death of neurons, in particular of midbrain dopaminergic neurons;
it protects dopaminergic neurons against energy deficit due to mitochondrial complex I inhibition, in particular against the mitochondrial neurotoxin MPP+;
it protects dopaminergic neurons against neurotrophic factor deprivation, in particular GDNF deprivation;
it protects neurons, in particular cortical neurons, from excitotoxicity, in particular against glutamate insult;
it protects cortical neurons from oxidative stress, in particular against H₂O₂ insult; and
it has an anti-inflammatory effect by limiting the number of microglial cells and astrocytes and/or by increasing levels of anti-inflammatory interleukin-10 (IL-10) levels when injected in vivo.

In one embodiment, the compound according to the invention increases intracellular NAADP levels, in particular in neurons, by at least 10%, 20%, 30%, 40%, 50%, particularly at least 60% and more particularly at least 70%, as compared to a negative control molecule, in an NAADP level measurement assay.

Methods for determining the intracellular NAADP levels are well-known in the art, such as by HPLC and Mass Spectrum analyses (see, Berridge et al., 2002. Biochem J. 365(Pt 1):295-301).

In particular, the compound according to the invention increases intracellular NAADP levels, in particular in neurons, by inhibiting the NAADP hydrolase activity of CD38 (particularly of human CD38) or by activating the NAADP synthase activity of CD38 (particularly of human CD38).

In particular, the compound according to the present invention increases intracellular NAADP levels, in particular in neurons, by inhibiting the ability of CD38 (particularly of human CD38) to degrade NAADP (in particular, into ADPRP) (i.e., by inhibiting the NAADP hydrolase activity of CD38) or by activating the ability of CD38 (particularly of human CD38) to synthetize NAADP (in particular, from NAM⁺, in the presence of nicotinic acid) (i.e., by activating the NAADP synthase activity of CD38).

The ability of a compound to inhibit the NAADP hydrolase activity of CD38 (i.e., to inhibit the ability of CD38 to degrade NAADP, in particular, into ADPRP) can be assayed using a method well-known in the art, such as in vitro using a solution comprising at least NAADP and CD38 (from recombinant CD38 proteins, extracted cellular proteins or whole cells expressing CD38; biochemical or cell-based assay) as described in Schmid et al. (2011. FEBS Lett. 585(22):3544-8).

The ability of a compound to activate the NAADP synthase activity of CD38 (i.e., to activate the ability of CD38 to synthetize NAADP, in particular, from NADP⁺ in the presence of nicotinic acid; NAADP synthase activity) can be assayed using a method well-known in the art, such as in vitro using a solution comprising at least NADP, nicotinic acid and CD38 (from recombinant CD38 proteins, extracted cellular proteins or whole cells expressing CD38; biochemical or cell-based assay) as described in Schmid et al. (2011. FEBS Lett. 585(22):3544-8).

In an embodiment, the compound according to the invention increases cytosolic calcium levels, in particular in neurons, by at least 10%, 20%, particularly at least 30% and more particularly at least 40%, as compared to a negative control molecule, in an intracellular calcium level measurement assay.

Methods for determining the cytosolic calcium levels are well-known in the art, such as by microscopy using fluorescent calcium indicator as will be further described in the Examples section below.

Several articles demonstrated that CD38 binding with certain antibodies triggered CD38 internalization and subsequent relocalization to the lysosome (Funaro et al., 1998. J Immunol. 160(5):2238-47). Moreover, NAADP synthesis by CD38 only occurs at acidic pH (Aarhus et al., 1995. J Biol Chem. 270(51):30327-33). Such pH conditions can only be found intracellularly in the lysosomal compartment. Fang et al. (2018. J Biol Chem. 293(21):8151-8160) have reported that upon binding of antibodies to CD38, the protein is internalized via clathrin-dependent endocytosis into lysosomal compartments. Fang et al. also found that the relocalization of CD38 into lysosomes increases intracellular NAADP level.

In one embodiment, the compound according to the invention triggers, upon its binding to CD38, the endocytic internalization of CD38. In one embodiment, the compound according to the invention triggers, upon its binding to CD38, the endocytic internalization of CD38 into clathrin-coated vesicles. In one embodiment, the compound according to the invention triggers, upon its binding to CD38, the endocytic internalization of CD38 into lysosome.

In one embodiment, the compound according to the invention is internalized in an endocytic compartment.

Methods to determine endocytic internalization of a particular cellular component (such as, e.g., CD38) or of an exogenously supplied compound (such as, e.g., the compound according to the present invention) are well-known in the art and include, for example, labelling and microscopic observation of said component or compound, quantification of the degree of protection from protease of said component or compound. Co-labeling with early endocytic markers such as for example, RabS or clathrin, or with lysosomal marker such as for example, lysotracker or LAMP1, may also be used to identify the endocytic compartment in which internalized CD38 is localized.

In one embodiment, the neuroprotective effect of the compound according to the invention is antagonized by at least 20%, 30%, 40%, 50%, particularly at least 60% and more particularly at least 70% in the presence of an endocytic internalization inhibitor when compared to a control without said inhibitor in a neuroprotective assay as will be further described in the Examples section below. A non-limiting example of an endocytic internalization inhibitor that can be used in such assay is jasplakinolide.

In one embodiment, the increase of intracellular calcium concentrations due to the compound according to the invention is antagonized by at least 5%, 10%, 15%, particularly at least 20% and more particularly at least 25% in the presence of an endocytic internalization inhibitor when compared to a control without such inhibitor in an intracellular calcium level measurement assay as will be further described in the Examples section below. A non-limiting example of an endocytic internalization inhibitor that can be used in such assay is jasplakinolide.

In one embodiment, the compound according to the invention triggers, upon binding to CD38, the endocytic internalization of CD38 in an endolysosomal, preferably a lysosomal compartment.

In one embodiment, the compound according to the invention is endocytosed in a lysosomal compartment.

In one embodiment, the neuroprotective effect of the compound according to the invention is antagonized by at least 20%, 30%, 40%, 50%, particularly at least 60% and more particularly at least 70% in the presence of a lysosomal acidification inhibitor when compared to a control without such inhibitor in a neuroprotective assay as will be further described in the Examples section below. A non-limiting example of a lysosomal acidification inhibitor that can be used in such assay is bafilomycin A1.

In one embodiment, the neuroprotective effect of the compound according to the invention is antagonized by at least 20%, 30%, 40%, 50%, particularly at least 60% and more particularly at least 70% in the presence of a lysosomal maturation and/or Ca²⁺-dependent exocytosis inhibitor when compared to a control without such inhibitor in a neuroprotective assay as will be further described in the Examples section below. Non-limiting examples of lysosomal maturation and/or Ca²⁺-dependent exocytosis inhibitors that can be used in such assay are vacuolin-1 and endosidin2.

In one embodiment, the neuroprotective effect of the compound according to the invention is not antagonized by more than 50%, 40%, 30%, 20%, 15%, particularly not more than 10% and more particularly not more than 5% in the presence of a sirtuin-1 inhibitor when compared to a control without such inhibitor in a neuroprotective assay as will be further described in the Examples section below. A non-limiting example of a sirtuin-1 inhibitor that can be used in such assay is Ex-527.

In one embodiment, the neuroprotective effect of the compound according to the invention is not antagonized by more than 50%, 40%, 30%, 20%, 15%, particularly not more than 10% and more particularly not more than 5% in the presence of a ryanodine receptor inhibitor when compared to a control without such inhibitor in a neuroprotective assay as will be further described in the Examples section below. A non-limiting example of a ryanodine receptor inhibitor that can be used in such assay is dantrolene.

In one embodiment, the compound according to the invention increases glucose uptake, in particular in neurons, by at least 30%, 40%, 50%, particularly at least 60% and more particularly at least 65%, as compared to a negative control molecule, in a glucose uptake assay as will be further described in the Examples section below.

In one embodiment, the compound according to the invention is selected from the group comprising or consisting of an anti-CD38 antibody, an antigen-binding fragment thereof and an antigen-binding antibody mimetic.

In particular, said anti-CD38 antibody or antigen-binding fragment thereof or antigen-binding antibody mimetic according to the invention specifically binds to the peptide comprising or consisting of amino acids 70 to 300, more particularly to the peptide comprising or consisting of amino acids 220 to 285 of human CD38 with SEQ ID NO: 1.

The numbering of amino acids of human CD38 as described herein corresponds to the numbering of amino acids of the human CD38 sequence set forth in SEQ ID NO: 1 and referenced by the NP_001766 NCBI accession number. Thus, said anti-CD38 antibody or antigen-binding fragment thereof or antigen-binding antibody mimetic according to the invention, which specifically bind to the peptide comprising or consisting of amino acids 70 to 300, more particularly to the peptide comprising or consisting of amino acids 220 to 285 of human CD38, specifically bind to the peptide comprising or consisting of amino acids at position 70 to 300, more particularly to the peptide comprising or consisting of amino acids 220 to 285 in the human CD38 sequence set forth in SEQ ID NO: 1 and referenced by the NP_001766 NCBI accession number.

In one embodiment, said anti-CD38 antibody or antigen-binding fragment thereof or antigen-binding antibody mimetic according to the invention specifically binds to cysteine 254 and/or cysteine 275 of human CD38 with SEQ ID NO: 1.

In one embodiment, said anti-CD38 antibody or antigen-binding fragment thereof or antigen-binding antibody mimetic according to the invention specifically binds to a region comprising the 5th (penultimate) C-terminal disulfide loop involving cysteine 254 and cysteine 275 of human CD38, in particular said region comprising or consisting of amino acids 220 to 285 of human CD38 with SEQ ID NO: 1.

In one embodiment, said anti-CD38 antibody or antigen-binding fragment thereof or antigen-binding antibody mimetic according to the invention specifically binds to the 5^{th} C-terminal disulfide loop involving cysteine 254 and cysteine 275 of human CD38 with SEQ ID NO: 1.

In one embodiment, said anti-CD38 antibody or antigen-binding fragment thereof or antigen-binding antibody mimetic according to the invention does not specifically bind to a region comprising the 6^{th} C-terminal disulfide loop involving cysteine 287 and cysteine 296 of human CD38 with SEQ ID NO: 1, more particularly said region comprising or consisting of amino acids 285 to 300 of human CD38 with SEQ ID NO: 1.

In one embodiment, said anti-CD38 antibody or antigen-binding fragment thereof or antigen-binding antibody mimetic according to the invention does not specifically bind to the 6^{th} C-terminal disulfide loop involving cysteine 287 and cysteine 296 of human CD38 with SEQ ID NO: 1.

In one embodiment, the anti-CD38 antibody according to the invention is selected in the group comprising or consisting of HB7, clone 90, AT1, AT13/5 (as described in Table 1) and mutated, recombinant (including chimeric and humanized), bispecific and modified antibodies thereof which are able to specifically bind to CD38 and activate the opening of NAADP receptors Two Pore Channels TPC1 and/or TPC2; particularly in the group comprising or consisting of HB7, clone 90, AT1 and mutated, recombinant (including chimeric and humanized), bispecific and modified antibodies thereof which are able to specifically bind to CD38 and activate the opening of NAADP receptors Two Pore Channels TPC1 and/or TPC2; more particularly in the group comprising or consisting of HB7, clone 90 and mutated, recombinant (including chimeric and humanized), bispecific and modified antibodies thereof which are able to specifically bind to CD38 and activate the opening of NAADP receptors Two Pore Channels TPC1 and/or TPC2; and even more particularly in the group comprising or consisting of HB7 and mutated, recombinant (including chimeric and humanized), bispecific and modified antibodies thereof which are able to specifically bind to CD38 and activate the opening of NAADP receptors Two Pore Channels TPC1 and/or TPC2.

In one embodiment, the anti-human CD38 antibody of the invention is a monoclonal antibody, in particular a humanized monoclonal antibody, more particularly wherein the antibody light chain constant domain is derived from a human kappa light chain constant domain and wherein the antibody heavy chain constant domain is derived from a human IgG1, IgG2, IgG3 or IgG4 (wild type or mutated) heavy chain constant domain. The humanized antibody or antigen-binding fragment thereof according to the invention has the advantage to be less immunogenic (or completely non-immunogenic) than murine versions in human subject to which they are administered.

As well-known by one skilled in the art, the choice of IgG isotypes of the heavy chain constant domain centers on whether specific functions are required and the need for a suitable in vivo half-life. For example, antibodies designed for selective eradication of cancer cells typically require an active isotype that permits complement activation and effector-mediated cell killing by antibody-dependent cell-mediated cytotoxicity. Both human IgG1 and IgG3 (shorter half-life) isotypes meet these criteria, particularly human IgG1 isotype (wild type and variants). In particular, depending of the IgG isotype of the heavy chain constant domain (particularly human wild type and variants IgG1 isotype), the antibody can be cytotoxic towards cells via a CDC, ADCC and/or ADCP mechanism (Salfeld, 2007. Nat Biotechnol. 25(12):1369-72; Irani et al., 2015. Mol Immunol. 67(2 Pt A):171-82). In fact, the fragment crystallizable (Fc) region interacts with a variety of accessory molecules to mediate indirect effector functions such as antibody-dependent cellular cytotoxicity (ADCC), antibody-dependent cellular phagocytosis (ADCP) and complement-dependent cytotoxicity (CDC).

In one embodiment, the antibody, antigen-binding fragment thereof or antigen-binding antibody mimetic is modified to facilitate delivery across the blood-brain barrier (BBB). Means and methods to modify antibodies to facilitate their crossing through the BBB, e.g., when parentally administered, are well-known in the art, and are described in, e.g., Yu et al., 2011. Sci Transl Med. 3(84):84ra44; Atwal et al., 2011. Sci Transl Med. 3(84):84ra43; and International patent applications WO2015031673 and WO2016208695.

As used herein, the term “small organic molecule” refers to a low molecular weight organic compound (up to 5000 Da, more in particular up to 2000 Da, and most in particular up to about 1000 Da).

In one embodiment, the small organic molecule according to the invention is selected in the group comprising or consisting of ara-2′-F-NAD⁺ (also named as ARA-F-NAD, (3-ara-2′-deoxy-2′-fluoro-nicotinamide adenine dinucleotide) and mutated small organic molecules thereof which are able to specifically bind to CD38 and activates the opening of NAADP receptors Two Pore Channels TPC1 and/or TPC2.kwong et al.

In one embodiment, the small organic molecule according to the invention is selected in the group comprising or consisting of those described in Kwong et al. (2012. Biochemistry. 51(1):555-64, which is incorporated herein by reference.

In one embodiment, the small organic molecule according to the invention is selected from ara-2′F-NMN and derivatives thereof such as for example:

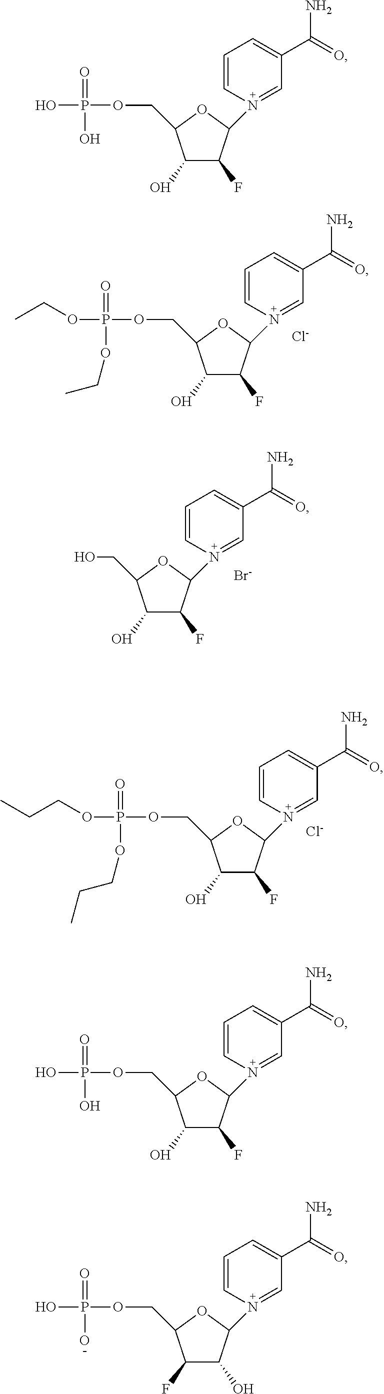

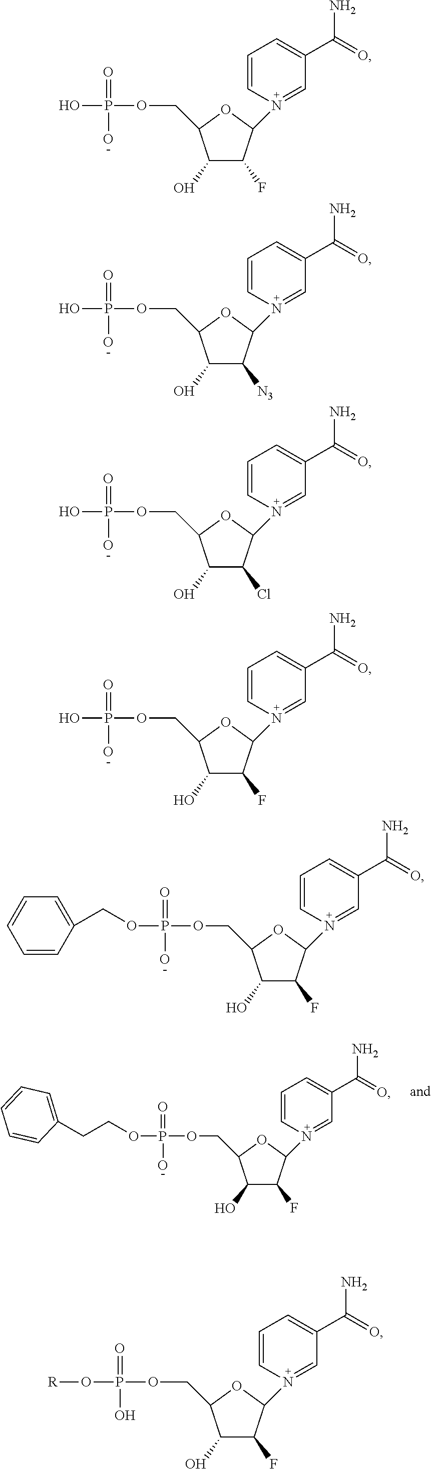

wherein R is selected from C₃H₇, C₄H₉, C₆H₁₃ or C₁₂H₂₅, and derivatives thereof.

In one embodiment, the small organic molecule according to the invention is selected in the group comprising or consisting of those described in Zhou et al. (2012. ChemMedChem. 7(2):223-8, which is incorporated herein by reference.

In one embodiment, the small organic molecule according to the invention is selected from

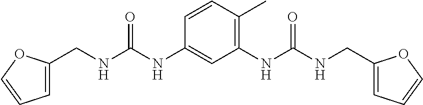 [SMILES: Cc1ccc(NC(=O)NCc2ccco2)cc1NC(=O)NCc1ccco1]

and derivative thereof such as for example:

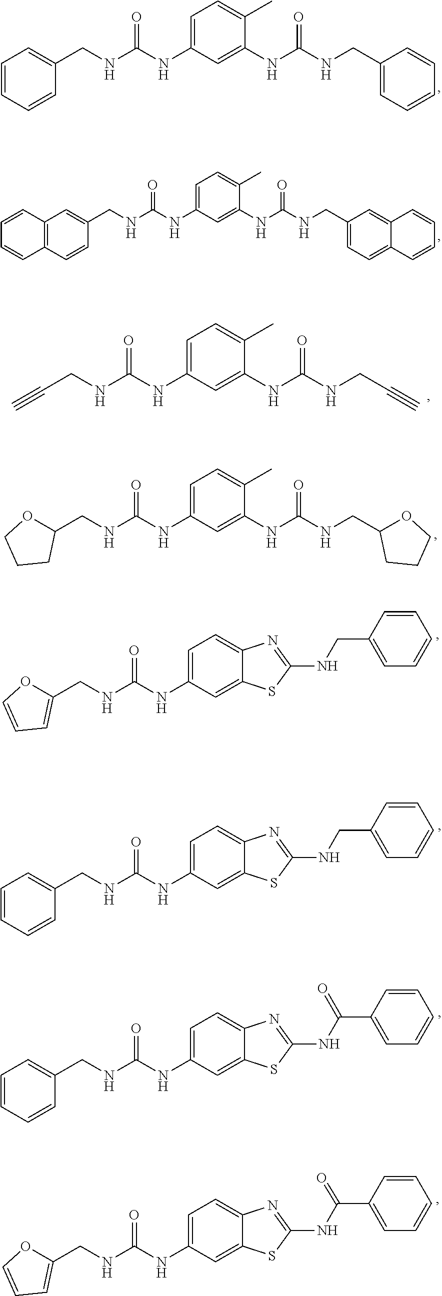

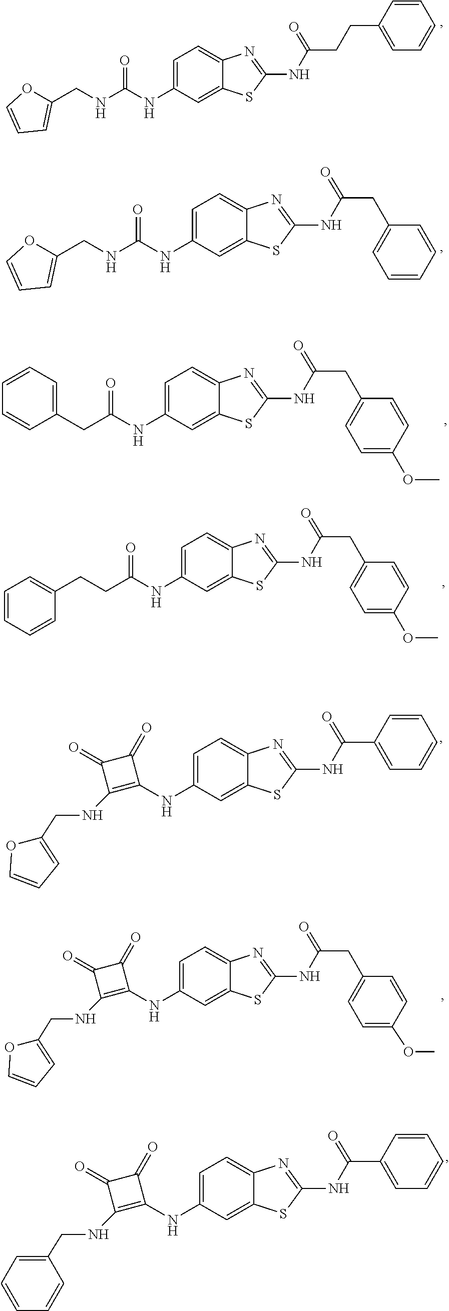

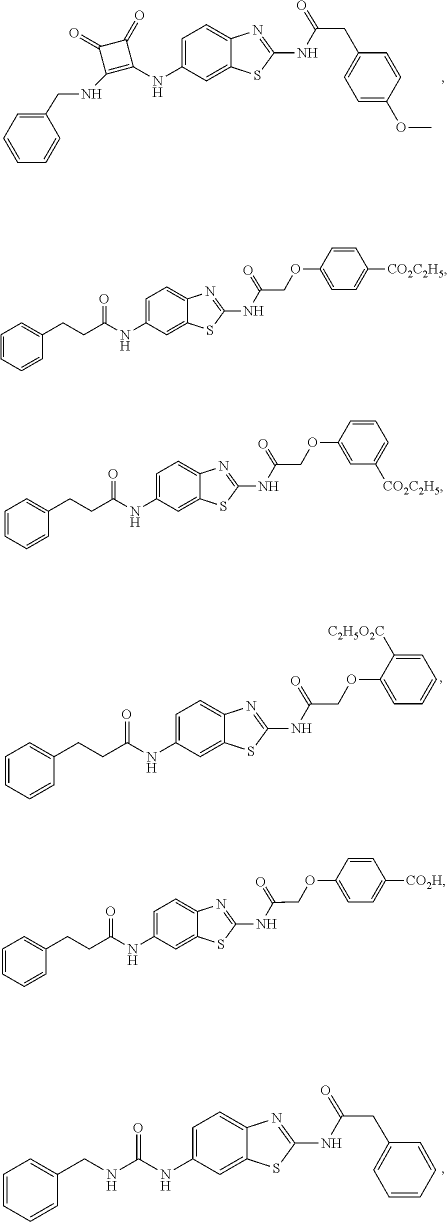

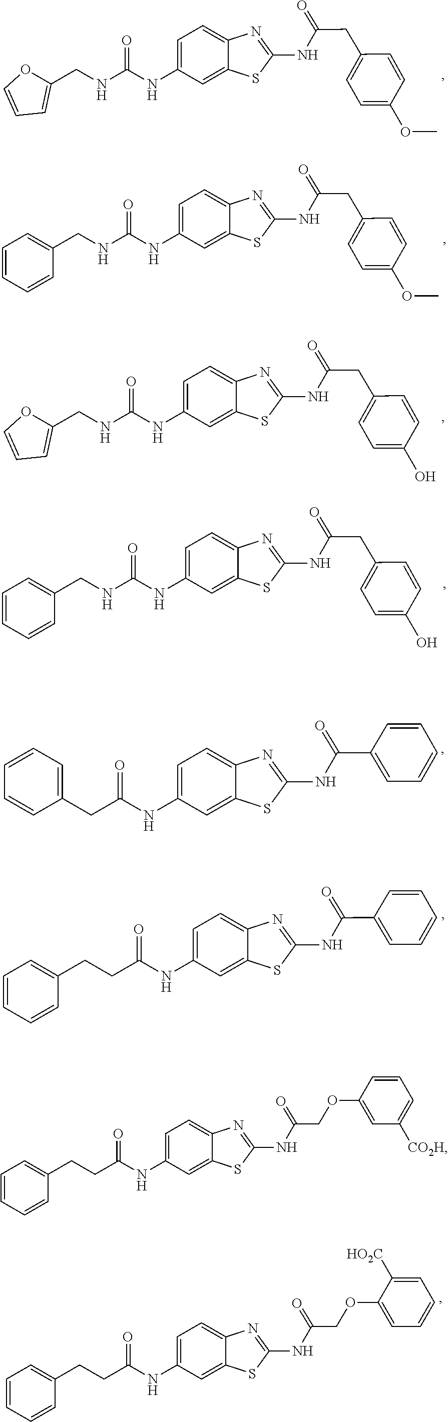

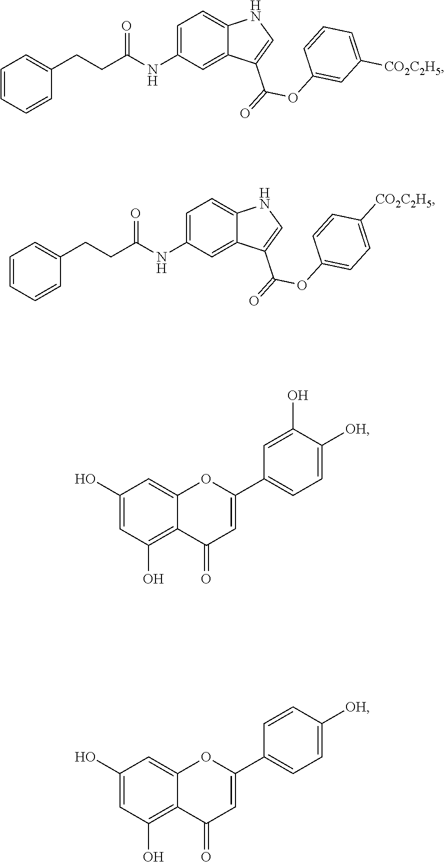 [SMILES: CCOC(=O)c1cccc(OC(=O)c2c[nH]c3ccc(NC(=O)CCc4ccccc4)cc23)c1 ; CCOC(=O)c1ccc(OC(=O)c2c[nH]c3ccc(NC(=O)CCc4ccccc4)cc23)cc1 ; O=c1cc(-c2ccc(O)c(O)c2)oc2cc(O)cc(O)c12 ; O=c1cc(-c2ccc(O)cc2)oc2cc(O)cc(O)c12]

and derivatives thereof.

In one embodiment, the small organic molecule according to the invention is selected in the group comprising or consisting of those described in Wu et al. (2013. Acta Pharm Sin B. 3(4):245-53), which is incorporated herein by reference.

In one embodiment, the small organic molecule according to the invention is selected from indole derivatives such as for example: (S)-4-(Ethoxycarbonyl)phenyl-5-[5-amino-2-(tert-butoxycarbonylamino)-5-oxopentanamido]-1H-indole-3-carboxylate; 4-(Ethoxycarbonyl)phenyl-5-propionamido-1H-indole-3-carboxylate; 4-(Ethoxycarbonyl)phenyl-5-(2-phenylacetamido)-1H-indole-3-carboxylate; 4-(Ethoxycarbonyl)phenyl-5-[2-(4-methoxyphenyl)acetami-do]-1H-indole-3-carboxylate; 4-(Ethoxycarbonyl)phenyl-5-(4-phenylbutanamido)-1H-indole-3-carboxylate; 4-(Ethoxycarbonyl)phenyl-5-[3-(pyridin-3-yl)propanamido]-1H-indole-3-carboxylate; 4-(Ethoxycarbonyl)phenyl-5-[3-(pyridin-4-yl)propanamido]-1H-indole-3-carboxylate; 4-(Ethoxycarbonyl)phenyl-5-[3-(furan-2-yl)propanamido]-1H-indole-3-carboxylate; 4-(Ethoxycarbonyl)phenyl-5-[5-oxo-5-(thiophen-2-yl)pentan-amido]-1H-indole-3-carboxylate; (S)-4-(Ethoxycarbonyl)phenyl-5-[2-(tert-butoxycarbonylamino)-3-(1H-imidazol-4-yl)-propanamido]-1H-indole-3-carboxylate; (S)-4-(Ethoxycarbonyl)phenyl-5-[2-(tert-butoxycarbonylamino)-3-(1H-indol-3-yl)-propanamido]-1H-indole-3-carboxylate; 4-(Ethoxycarbonyl)phenyl-5-[3-(4-chlorophenyl)propanam-ido]-1H-indole-3-carboxylate; 4-(Ethoxycarbonyl)phenyl-5-[3-(3-chlorophenyl)propanamido]-1H-indole-3-carboxylate; 4-(Ethoxycarbonyl)phenyl-5-[3-(4-fluorophenyl)propanamido]-1H-indole-3-carboxylate; 4-(Ethoxycarbonyl)phenyl-5-[3-(2-fluorophenyl)propanamido]-1H-indole-3-carboxylate; 4-(Ethoxycarbonyl)phenyl-5-{3-[3-(trifluoromethyl)phenyl]propanamido}-1H-indole-3-carboxylate; 4-(Ethoxycarbonyl)phenyl-5-[3-(4-nitrophenyl)propanamido]-1H-indole-3-carboxylate; 4-(Ethoxycarbonyl)phenyl-5-[3-(3-nitrophenyl)propanamido]-1H-indole-3-carboxylate; 4-(Ethoxycarbonyl)phenyl-5-[3-(4-methoxyphenyl)propanamido]-1H-indole-3-carboxylate; 4-(Ethoxycarbonyl)phenyl-5-[3-(3,4,5-trimethoxyphenyl)propanamido]-1H-indole-3-carboxylate; 1-(3-(3-{[4-(Ethoxycarbonyl)phenoxy]carbonyl}-1H-indol-5-ylamino)-3-oxopropyl)pyridinium chloride; 3-Carbamoyl-1-(3-(3-{[4-(ethoxycarbonyl)phenoxy]carbonyl}-1H-indol-5-ylamino)-3-oxopropyl)pyridinium chloride; 4-Carbamoyl-1-(3-(3-{[4-(ethoxycarbonyl)phenoxy]carbonyl}-1H-indol-5-ylamino)-3-oxopropyl)pyridinium chloride; (S)-4-(Ethoxycarbonyl)phenyl-5-(2-amino-3-phenylpropanamido)-1H-indole-3-carboxylate; (R)-4-(Ethoxycarbonyl)phenyl-5-(2-amino-3-phenylpropanamido)-1H-indole-3-carboxylate; 4-(Ethoxycarbonyl)phenyl-5-[3-(4-aminophenyl)propanamido]-1H-indole-3-carboxylate; 4-(Ethoxycarbonyl)phenyl-5-[3-(3-aminophenyl)propanamido]-1H-indole-3-carboxylate; 4-(Ethoxycarbonyl)phenyl-5-{3-[3-(4-hydroxybutylamino)phenyl]propanamido}-1H-indole-3-carboxylate; 4-(Ethoxycarbonyl)phenyl-5-(4-hydroxybutylamino)-1H-indole-3-carboxylate; and derivatives thereof.

In one embodiment, the small organic molecule according to the invention is selected in the group comprising or consisting of those described in Wang et al. (2014. Molecules. 19: 15754-67), which is incorporated herein by reference.

In one embodiment, the small organic molecule according to the invention is selected from NAD analogues and derivatives such as for example: 3,5-Di-O-benzoyl-2-deoxy-2-fluoro-2-methyl-1-bromide-D-ribofuranose; 2′-Deoxy-2′-fluoro-2′-methyl-β-nicotinamide ribofuranoside; 2′-Deoxy-2′-fluoro-2′-methyl-β-nicotinamide mononucleotide; 2′-Deoxy-2′-fluoro-2′-methyl-β-nicotinamide adenine dinucleotide; 2′-Deoxy-2′-fluoro-5′-thio-phosphate-arabinofuranoside-β-nicotinamide adenine dinucleotide; P1-(Adenosine)-P3-(2′-deoxy-2′-fluoro-β-nicotinamide arabinofuranoside) triphosphate; 2′-Deoxy-2′-fluoro-arabinosyl-β-nicotinamide hypoxanthine dinucleotide; 2′-Deoxy-2′-fluoro-arabinosyl-β-nicotinamide guanine dinucleotide; 2′-Deoxy-2′-fluoro-arabinosyl-β-nicotinamide 6-O-methyl-hypoxanthine dinucleotide; Bis(2′-deoxy-2′-fluoro-β-nicotinamide-arabinosyl) pyrophosphate; Bis(2′-deoxy-2′-fluoro-β-nicotinamide-arabinosyl)-methylenediphosphonate; Bis(2′-deoxy-2′-fluoro-2′-methyl-β-nicotinamide)-methylenediphosphonate and derivatives thereof.

In one embodiment, the small organic molecule according to the invention is selected in the group comprising or consisting of those described in Swarbrick et al. (2014. J Med Chem. 57(20): 8517-29), which is incorporated herein by reference.

In one embodiment, the small organic molecule according to the invention is selected from the group comprising or consisting of:

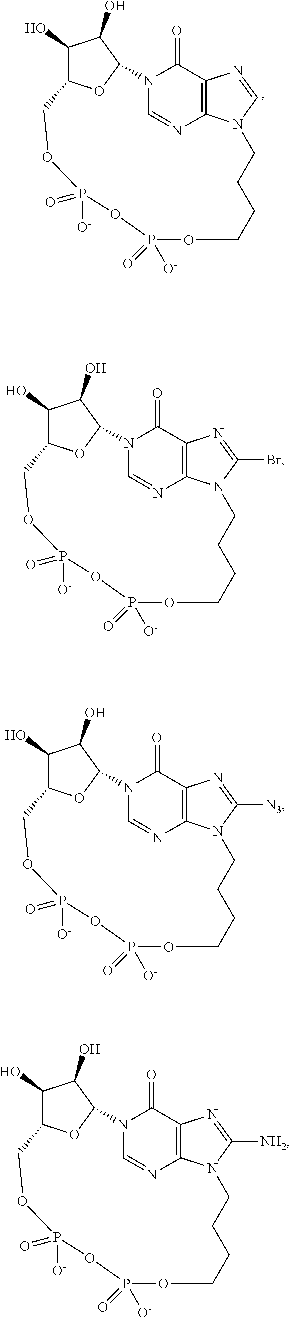 [SMILES: O=c1c2ncn3c2ncn1[C@@H]1O[C@H](COP(=O)([O-])OP(=O)([O-])OCCCC3)[C@@H](O)[C@H]1O ; O=c1c2nc(Br)n3c2ncn1[C@@H]1O[C@H](COP(=O)([O-])OP(=O)([O-])OCCCC3)[C@@H](O)[C@H]1O ; [N-]=[N+]=Nc1nc2c(=O)n3cnc2n1CCCCOP(=O)([O-])OP(=O)([O-])OC[C@H]1O[C@@H]3[C@H](O)[C@@H]1O ; Nc1nc2c(=O)n3cnc2n1CCCCOP(=O)([O-])OP(=O)([O-])OC[C@H]1O[C@@H]3[C@H](O)[C@@H]1O]

and derivatives thereof.

In one embodiment, the small organic molecule according to the invention is selected in the group comprising or consisting of those described in Becherer et al. (2015. J Med Chem. 58(17):7021-56, which is incorporated herein by reference.

In one embodiment, the small organic molecule according to the invention is selected from 4-amino-8quinoline carboxamides such as for example: 8-Bromo-N-[(2,6-dimethylphenyl)methyl]-2-methyl-4-quinolinamine; 4-{[(2,6-Dimethylphenyl)methyl]amino}-2-methyl-8-quinolinecarboxamide; 4-Amino-2-methyl-8-quinolinecarboxamide; N-Benzyl-8-bromo-2-methylquinolin-4-amine; 4-(Benzylamino)-2-methylquinoline-8-carbonitrile; 4-(Benzylamino)-2-methylquinoline-8-carboxamide; 4-Hydroxy-2-methylquinoline-8-carbonitrile; 8-Cyano-2-methylquinolin-4-yl trifluoromethanesulfonate; 2-Methyl-4-(2-methylbenzylamino)quinoline-8-carbonitrile; 2-Methyl-4-(2-methylbenzylamino)quinoline-8-carboxamide; 4-(2-Chlorobenzylamino)-2-methylquinoline-8-carbonitrile; 2-Methyl-4-(2-methylbenzylamino)quinoline-8-carboxamide; 8-Bromo-N-(2-methoxybenzyl)-2-methylquinolin-4-amine; 4-((2-Methoxybenzyl)amino)-2-methylquinoline-8-carboxamide; 8-Bromo-2-methyl-N-(3-methylbenzyl)quinolin-4-amine; 2-Methyl-4-((3-methylbenzyl)amino)quinoline-8-carboxamide; 8-Bromo-N-[(3-chlorophenyl)methyl]-2-methyl-4-quinolinamine; 4-{1[(3-Chlorophenyl)methyl]amino}-2-methyl-8-quinolinecarboxamide; 8-Bromo-N-(3-methoxybenzyl)-2-methylquinolin-4-amine; 4-((3-Methoxybenzyl)amino)-2-methylquinoline-8-carboxamide; 8-Bromo-2-methyl-N-(4-methylbenzyl)quinolin-4-amine; 2-Methyl-4-((4-methylbenzyl)amino)quinoline-8-carboxamide; 4-(4-Chlorobenzylamino)-2-methylquinoline-8-carbonitrile; 4-(4-Chlorobenzylamino)-2-methylquinoline-8-carboxamide; 8-Bromo-N-(4-methoxybenzyl)-2-methylquinolin-4-amine; 4-((4-Methoxybenzyl)amino)-2-methylquinoline-8-carboxamide; 2-Methyl-4-(2-(trifluoromethyl)benzylamino)quinoline-8-carbonitrile; 2-Methyl-4-(2-(trifluoromethyl)benzylamino)quinoline-8-carboxamide; 4-(2-Bromobenzylamino)-2-methylquinoline-8-carbonitrile; 4-(2-Bromobenzylamino)-2-methylquinoline-8-carboxamide; 4-({[2,4-Bis(methyloxy)phenyl]methyl}amino)-2-methyl-8-quinolinecarbonitrile; 4-Amino-2-methyl-8-quinolinecarbonitrile; 4-(2-Cyanobenzylamino)-2-methylquinoline-8-carbonitrile; 4-(2-Carbamoylbenzylamino)-2-methylquinoline-8-carboxamide; 4-(2,3-Dimethylbenzylamino)-2-methylquinoline-8-carbonitrile; (2,3-Dimethylbenzylamino)-2-methylquinoline-8-carboxamide trifluoroacetate; 4-{[(2,3-Dichlorophenyl)methyl]amino}-2-methyl-8-quinolinecarboxamide; 4-(2,4-Dimethylbenzylamino)-2-methylquinoline-8-carbonitrile; 4-(2,4-Dimethylbenzylamino)-2-methylquinoline-8-carboxamide trifluoroacetate; 4-(2,4-Dichlorobenzylamino)-2-methylquinoline-8-carbonitrile; 4-(2,4-Dichlorobenzylamino)-2-methylquinoline-8-carboxamide; 8-Bromo-N-(2,5-dimethylbenzyl)-2-methylquinolin-4-amine; 4-(2,5-Dimethylbenzylamino)-2-methylquinoline-8-carbonitrile; 4-(2,5-Dimethylbenzylamino)-2-methylquinoline-8-carboxamide; 4-(2,5-Dichlorobenzylamino)-2-methylquinoline-8-carbonitrile; 2-Methyl-4-(2-methylbenzylamino)quinoline-8-carboxamide; 4-(2,6-Dichlorobenzylamino)-2-methylquinoline-8-carbonitrile; 4-(2,6-Dichlorobenzylamino)-2-methylquinoline-8-carboxamide; 4-(3,4-Dimethylbenzylamino)-2-methylquinoline-8-carbonitrile; 4-(3,4-Dimethylbenzylamino)-2-methylquinoline-8-carboxamide; 4-(3,4-Dichlorobenzylamino)-2-methylquinoline-8-carbonitrile; 4-(3,4-Dichlorobenzylamino)-2-methylquinoline-8-carboxamide hydrochloride; 8-Bromo-N-(3,5-dimethylbenzyl)-2-methylquinolin-4-amine; 4-(3,5-Dimethylbenzylamino)-2-methylquinoline-8-carbonitrile; 4-(3,5-Dimethylbenzylamino)-2-methylquinoline-8-carboxamide; 4-(3,5-Dichlorobenzylamino)-2-methylquinoline-8-carbonitrile; 4-(3,5-Dichlorobenzylamino)-2-methylquinoline-8-carboxamide; 4-(3-Chloro-2-fluorobenzylamino)-2-methylquinoline-8-carbonitrile; 4-(3-Chloro-2-fluorobenzylamino)-2-methylquinoline-8-carboxamide; 4-(3-Chloro-2-methylbenzylamino)-2-methylquinoline-8-carbonitrile; 4-(3-Chloro-2-methylbenzylamino)-2-methylquinoline-8-carboxamide; 4-(2-Fluoro-3-(trifluoromethyl)benzylamino)-2-methylquinoline-8-carbonitrile; 4-(2-Fluoro-3-(trifluoromethyl)benzylamino)-2-methylquinoline-8-carboxamide; 2-Methyl-4-(naphthalen-1-ylmethylamino)quinoline-8-carbonitrile; 2-Methyl-4-(naphthalen-1-ylmethylamino)quinoline-8-carboxamide; 4-(2,6-Difluorobenzylamino)-2-methylquinoline-8-carbonitrile; 4-(2-Chloro-3,6-difluorobenzylamino)-2-methylquinoline-8-carboxamide; 4-(2-Chloro-6-fluorobenzylamino)-2-methylquinoline-8-carbonitrile; 4-(2-Chloro-6-fluorobenzylamino)-2-methylquinoline-8-carboxamide; 4-(2-Chloro-6-methylbenzylamino)-2-methylquinoline-8-carbonitrile; 4-(2-Chloro-6-methylbenzylamino)-2-methylquinoline-8-carboxamide; 8-Bromo-N-(2-fluoro-6-(trifluoromethyl)benzyl)-2-methylquinolin-4-amine; 4-((2-Fluoro-6-(trifluoromethyl)benzyl)amino)-2-methylquinoline-8-carboxamide; 8-Bromo-2-methyl-N-[(2,3,6-trichlorophenyl)methyl]-4-quinolinamine; 2-Methyl-4-{[(2,3,6-trichlorophenyl)methyl]amino}-8-quinolinecarboxamide; 4-{[(3-Chloro-2,6-difluorophenyl)methyl]amino}-2-methyl-8-quinolinecarbonitrile; 4-{1[(3-Chloro-2,6-difluorophenyl)methyl]amino}-2-methyl-8-quinolinecarboxamide; 4-(2-Chloro-3,6-difluorobenzylamino)-2-methylquinoline-8-carbonitrile; 4-(2-Chloro-3,6-difluorobenzylamino)-2-methylquinoline-8-carboxamide; 8-Bromo-N-[(3,6-dichloro-2-fluorophenyl)methyl]-2-methyl-4-quinolinamine; 4-{[(3,6-Dichloro-2-fluorophenyl)methyl]amino}-2-methyl-8-quinolinecarboxamide; 8-Bromo-N-[(2,3-dichloro-6-fluorophenyl)methyl]-2-methyl-4-quinolinamine; 4-{[(2,3-Dichloro-6-fluorophenyl)methyl]amino}-2-methyl-8-quinolinecarboxamide; 8-Bromo-N-[(2-chloro-6-fluoro-3-methylphenyl)methyl]-2-methyl-4-quinolinamine; 4-{[(2-Chloro-6-fluoro-3-methylphenyl)methyl]amino}-2-methyl-8-quinolinecarboxamide; 8-Bromo-2-methyl-N-{[3-(trifluoromethyl)-2-pyridinyl]methyl}-4-quinolinamine; 2-Methyl-4-({[3-(trifluoromethyl)-2-pyridinyl]methyl}amino)-8-quinolinecarboxamide; 8-Bromo-2-methyl-N-{[4-(trifluoromethyl)-3-pyridinyl]methyl}-4-quinolinamine; 2-Methyl-4-({[4-(trifluoromethyl)-3-pyridinyl]methyl}amino)-8-quinolinecarboxamide; 8-Bromo-N-[(3-chloro-4-pyridinyl)methyl]-2-methyl-4-quinolinamine; 4-{[(3-Chloro-4-pyridinyl)methyl]amino}-2-methyl-8-quinolinecarboxamide; 8-Bromo-2-methyl-N-{[2-(trifluoromethyl)-3-pyridinyl]methyl}-4-quinolinamine; 2-Methyl-4-({[2-(trifluoromethyl)-3-pyridinyl]methyl}amino)-8-quinolinecarboxamide; 8-Bromo-N-[(3-chloro-2-thienyl)methyl]-2-methyl-4-quinolinamine; 4-{[(3-Chloro-2-thienyl)methyl]amino}-2-methyl-8-quinolinecarboxamide; 8-Bromo-N-[(3,5-dimethyl-4-isoxazolyl)methyl]-2-methyl-4-quinolinamine; 4-{[(3,5-Dimethyl-4-isoxazolyl)methyl]amino}-2-methyl-8-quinolinecarboxamide; 8-Bromo-N-(cyclohexylmethyl)-2-methyl-4-quinolinamine; 4-[(Cyclohexylmethyl)amino]-2-methyl-8-quinolinecarboxamide; N-[(1-Acetyl-4-piperidinyl)methyl]-8-bromo-2-methyl-4-quinolinamine; 4-{[(1-Acetyl-4-piperidinyl)methyl]amino}-2-methyl-8-quinolinecarboxamide; 8-Bromo-2-methyl-N-[(1-methyl-4-piperidinyl)methyl]-4-quinolinamine; 2-Methyl-4-{[(1-methyl-4-piperidinyl)methyl]amino}-8-quinolinecarboxamide; 8-Bromo-2-methyl-N-[(1R)-1-(2-methylphenyl)ethyl]-4-quinolinamine; 2-Methyl-4-{[(1R)-1-(2-methylphenyl)ethyl]amino}-8-quinolinecarboxamide; 8-Bromo-N-[(1S)-1-(2,6-dimethylphenyl)ethyl]-2-methyl-4-quinolinamine; 4-{[(1S)-1-(2,6-Dimethylphenyl)ethyl]amino}-2-methyl-8-quinolinecarboxamide; 8-Bromo-2-methyl-N-[(1R)-1,2,3,4-tetrahydro-1-naphthalenyl]-4-quinolinamine; 2-Methyl-4-[(1R)-1,2,3,4-tetrahydro-1-naphthalenylamino]-8-quinolinecarboxamide; 8-Bromo-2-methyl-N-[(1S)-1,2,3,4-tetrahydro-1-naphthalenyl]-4-quinolinamine; 2-Methyl-4-[(1S)-1,2,3,4-tetrahydro-1-naphthalenylamino]-8-quinolinecarboxamide; 8-Bromo-N-(2,6-dichlorobenzyl)-N,2-dimethylquinolin-4-amine; 4-((2,6-Dichlorobenzyl)(methyl)amino)-2-methylquinoline-8-carboxamide; 2,6-Dichloro-N-(8-cyano-2-methyl-4-quinolinyl)benzamide; 4-(2,6-Dichlorobenzamido)-2-methylquinoline-8-carboxamide; 4-(2,6-Dichlorobenzyloxy)-2-methylquinoline-8-carbonitrile; 4-((2,6-Dichlorobenzyl)oxy)-2-methylquinoline-8-carboxamide; 8-Bromo-4-((2,6-dichlorobenzyl)thio)-2-methylquinoline; 4-((2,6-Dichlorobenzyl)thio)-2-methylquinoline-8-carboxamide; 8-Bromo-6-fluoro-2-methylquinolin-4-ol; 8-Bromo-4-chloro-6-fluoro-2-methylquinoline; 8-Bromo-6-fluoro-2-methyl-N-(2,3,6-trichlorobenzyl)quinolin-4-amine; 6-Fluoro-2-methyl-4-((2,3,6-trichlorobenzyl)amino)quinoline-8-carboxamide; Methyl 6-bromo-4-hydroxy-2-methylquinoline-8-carboxylate; Methyl 6-bromo-4-chloro-2-methyl-8-quinolinecarboxylate; Methyl 4-chloro-2,6-dimethyl-8-quinolinecarboxylate; 4-Chloro-2,6-dimethyl-8-quinolinecarboxamide; 4-{[(2,6-Dimethylphenyl)methyl]amino}-2,6-dimethyl-8-quinolinecarboxamide; 6-Bromo-4-chloro-2-methylquinoline-8-carboxamide; 6-Bromo-4-((2,6-dimethylbenzyl)amino)-2-methylquinoline-8-carboxamide; 4-Chloro-2-methyl-6-phenyl-8-quinolinecarboxamide; 4-{[(2,6-Dimethylphenyl)methyl]amino}-2-methyl-6-phenyl-8-quinolinecarboxamide; N-[(2,3-Dichlorophenyl)methyl]-2-methyl-4-quinolinamine; 8-Bromo-N-[(2,3-dichlorophenyl)methyl]-2-methyl-4-quinolinamine; 4-{[(2,3-Dichlorophenyl)methyl]amino}-N,2-dimethyl-8-quinolinecarboxamide; 4-{[(2,3-Dichlorophenyl)methyl]amino}-N,N,2-trimethyl-8-quinolinecarboxamide; Methyl 4-{[(2,3-dichlorophenyl)methyl]amino}-2-methyl-8-quinolinecarboxylate; 4-{[(2,3-Dichlorophenyl)methyl]amino}-2-methyl-8-quinolinecarboxylic acid; (4-{[(2,3-Dichlorophenyl)methyl]amino}-2-methyl-8-quinolinyl)methanol; 8-(Aminomethyl)-N-[(2,3-dichlorophenyl)methyl]-2-methyl-4-quinolinamine; 4-{[(2,3-Dichlorophenyl)methyl]amino}-2-methyl-8-quinolinecarbonitrile; 4-Chloro-2-methyl-8-quinolinesulfonamide; 4-{[(2,3-Dichlorophenyl)methyl]amino}-2-methyl-8-quinolinesulfonamide; and derivatives thereof.

In one embodiment, the small organic molecule according to the invention is selected in the group comprising or consisting of those described in Haffner et al. (2015. J Med Chem. 58(8): 3548-71, which is incorporated herein by reference.

In one embodiment, the small organic molecule according to the invention is selected from

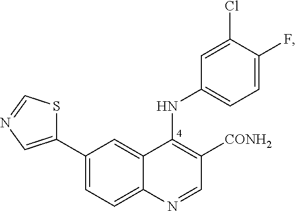 [SMILES: NC(=O)c1cnc2ccc(-c3cncs3)cc2c1Nc1ccc(F)c(Cl)c1]

and derivatives thereof such as for example: 6-Bromo-N-(3-chloro-4-fluorophenyl)-4-quinolinamine; N-(3-Chloro-4-fluorophenyl)-6-(1,3-thiazol-5-yl)-4-quinolinamine; 6-Bromo-N-phenylquinazolin-4-amine; N-phenyl-6-(thiazol-5-yl)quinazolin-4-amine; 2-Amino-5-bromo-3-methoxybenzoic acid; 2-Amino-5-bromo-3-(trifluoromethyl)benzoic acid; 2-Amino-5-iodo-3-(trifluoromethyl)benzoic acid; 6-Bromo-8-methoxyquinazolin-4(3H)-one; 6-Bromo-8-(trifluoromethyl)-4(1H)-quinazolinone; 6-Bromo-8-(trifluoromethyl)-4(1H)-quinazolinone; 6-Bromo-4-chloro-8-methoxyquinazoline; 6-Bromo-4-chloro-8-(trifluoromethyl)quinazoline; 6-Bromo-4-chloro-8-methylquinazoline; 6-Bromo-N-(3-chloro-4-fluorophenyl)-8-methoxyquinazolin-4-amine; 4-{[6-Bromo-8-(methyloxy)-4-quinazolinyl]amino}benzamide; 4-{[6-Bromo-8-(trifluoromethyl)-4-quinazolinyl]amino}benzamide; -Bromo-N-(3-chloro-4-fluorophenyl)-8-(trifluoromethyl)-4-quinazolinamine; 6-Bromo-N-(3-chloro-4-fluorophenyl)-8-methyl-4-quinazolinamine; N-(3-Chloro-4-fluorophenyl)-8-methoxy-6-(thiazol-5-yl)quinazolin-4-amine; 4-{[8-(Methyloxy)-6-(1,3-thiazol-5-yl)-4-quinazolinyl]amino}benzamide; 4-{[6-(1,3-Thiazol-5-yl)-8-(trifluoromethyl)-4-quinazolinyl]amino}benzamide; N-(3-Chloro-4-fluorophenyl)-6-(1,3-thiazol-5-yl)-8-(trifluoromethyl)-4-quinazolinamine; N-(3-Chloro-4-fluorophenyl)-8-methyl-6-(1,3-thiazol-5-yl)-4-quinazolinamine; 6-Bromo-8-methyl-2,4(1H,3H)-quinazolinedione; 4-Hydroxy-6-iodoquinazolin-2(1H)-one; 6-Bromo-2,4-dichloro-8-methylquinazoline; 2,4-Dichloro-6-iodoquinazoline; 6-Bromo-2-chloro-N-(3-chloro-4-fluorophenyl)-8-methyl-4-quinazolinamine; 6-Bromo-2-chloro-N-((1s,4s)-4-methoxycyclohexyl)-8-methylquinazolin-4-amine; 2-Chloro-6-iodo-N-((1s,4s)-4-(2-methoxyethoxy)cyclohexyl)quinazolin-4-amine; 2-Chloro-6-iodo-N-((1s,4s)-4-methoxycyclohexyl)quinazolin-4-amine; 6-Bromo-4-((3-chloro-4-fluorophenyl)amino)-8-methylquinazolin-2(1H)-one; 6-Bromo-4-(((1s,4s)-4-methoxycyclohexyl)amino)-8-methylquinazolin-2(1H)-one; 6-Iodo-4-(((1s,4s)-4-(2-methoxyethoxy)cyclohexyl)amino)quinazolin-2-ol; 6-Iodo-4-(((1s,4s)-4-methoxycyclohexyl)amino)quinazolin-2-ol; 4-((3-Chloro-4-fluorophenyl)amino)-8-methyl-6-(thiazol-5-yl)quinazolin-2(1H)-one; 4-(((1s,4s)-4-Methoxycyclohexyl)amino)-8-methyl-6-(thiazol-5-yl)quinazolin-2(1H)-one; 6-Iodo-4-(((1s,4s)-4-(2-methoxyethoxy)cyclohexyl)amino)-1-methylquinazolin-2(1H)-one; 6-Iodo-4-(((1s,4s)-4-methoxycyclohexyl)amino)-1-methylquinazolin-2(1H)-one; 4-(((1s,4s)-4-(2-Methoxyethoxy)cyclohexyl)amino)-1-methyl-6-(thiazol-5-yl)quinazolin-2(1H)-one; 4-(((1s,4s)-4-Methoxycyclohexyl)amino)-1-methyl-6-(thiazol-5-yl)quinazolin-2(1H)-one; 6-Iodo-2-methyl-8-(trifluoromethyl)quinazolin-4-ol; 6-Bromo-8-methyl-2-(trifluoromethyl)-4H-benzo[d][1,3]oxazin-4-one; 6-Bromo-8-methyl-2-(trifluoromethyl)quinazolin-4-(3H)-one; 4-Chloro-6-iodo-2-methyl-8-(trifluoromethyl)quinazoline; 6-Bromo-4-chloro-2,8-dimethylquinazoline; (1s,4s)-N-Methyl-4-((2-methyl-6-(thiazol-5-yl)-8-(trifluoromethyl)quinazolin-4-yl)amino)cyclohexanecarboxamide; (1s,4s)-4-((6-Bromo-8-methyl-2-(trifluoromethyl)quinazolin-4-yl)amino)-Nmethylcyclohexanecarboxamide; (1s,4s)-N-Methyl-4-((8-methyl-6-(thiazol-5-yl)-2-(trifluoromethyl)quinazolin-4-yl)amino)cyclohexanecarboxamide; (1s,4s)-4-((2,8-Dimethyl-6-(thiazol-5-yl)quinazolin-4-yl)amino)-Nmethylcyclohexanecarboxamide; 4-Iodo-N-methylaniline; 4-Iodo-N,2-dimethylaniline; Methyl 3-((4-iodophenyl)amino)-3-oxopropanoate; Methyl 3-((4-iodo-2-methylphenyl)amino)-3-oxopropanoate; Methyl 3-((4-iodophenyl)(methyl)amino)-3-oxopropanoate; 3-((4-Iodophenyl)amino)-3-oxopropanoic acid; 3-((4-Iodo-2-methylphenyl)amino)-3-oxopropanoic acid; 3-((4-Iodophenyl)(methyl)amino)-3-oxopropanoic acid; 3-((4-Iodo-2-methylphenyl)(methyl)amino)-3-oxopropanoic acid; 6-Iodoquinoline-2,4(1H,3H)-dione; 6-Iodo-8-methylquinoline-2,4-diol; 4-Hydroxy-6-iodo-1-methylquinolin-2(1H)-one; 4-Hydroxy-6-iodo-1,8-dimethylquinolin-2(1H)-one; 2,4-Dichloro-6-iodoquinoline; 2,4-Dichloro-6-iodo-8-methylquinoline; 4-Chloro-6-iodoquinolin-2(1H)-one; 6-Bromo-4-chloro-8-methylquinolin-2(1H)-one; 4-Chloro-6-iodo-8-methylquinolin-2(1H)-one; (1s,4s)-4-((6-Bromo-2-oxo-1,2-dihydroquinolin-4-yl)amino)-N-methylcyclohexanecarboxamide; 6-Bromo-4-((tetrahydro-2H-pyran-4-yl)amino)quinolin-2(1H)-one; 6-Bromo-4-(((1r,4r)-4-(2-methoxyethoxy)cyclohexyl)amino)quinolin-2(1H)-one; 6-Bromo-4-(((1r,4r)-4-(2-methoxyethoxy)cyclohexyl)amino)-8-methylquinolin-2(1H)-one; 4-Chloro-6-iodo-1-methylquinolin-2(1H)-one; -Chloro-6-iodo-1,8-dimethylquinolin-2(1H)-one; 4-Chloro-6-(thiazol-5-yl)quinolin-2(1H)-one; 4-Chloro-8-methyl-6-(thiazol-5-yl)quinolin-2(1H)-one; 4-Chloro-1-methyl-6-(thiazol-5-yl)quinolin-2(1H)-one; 4-Chloro-1,8-dimethyl-6-(thiazol-5-yl)quinolin-2(1H)-one; 4-Chloro-1-ethyl-6-(thiazol-5-yl)quinolin-2(1H)-one; (1s,4s)-N-Methyl-4-((2-oxo-6-(thiazol-5-yl)-1,2-dihydroquinolin-4-yl)amino)cyclohexanecarboxamide; 4-((Tetrahydro-2H-pyran-4-yl)amino)-6-(thiazol-5-yl)quinolin-2(1H)-one; 4-(((1s,4s)-4-(2-Methoxyethoxy)cyclohexyl)amino)-6-(thiazol-5-yl)quinolin-2(1H)-one; (1r,4r)-N-Methyl-4-((8-methyl-2-oxo-6-(thiazol-5-yl)-1,2-dihydroquinolin-4-yl)amino)cyclohexanecarboxamide; 8-Methyl-4-((tetrahydro-2H-pyran-4-yl)amino)-6-(thiazol-5-yl)quinolin-2(1H)-one; 4-(((1s,4s)-4-(2-Methoxyethoxy)cyclohexyl)amino)-8-methyl-6-(thiazol-5-yl)quinolin-2(1H)-one; 4-(((1s,4s)-4-Methoxycyclohexyl)amino)-8-methyl-6-(thiazol-5-yl)quinolin-2(1H)-one; (1r,4r)-N-Methyl-4-((1-methyl-2-oxo-6-(thiazol-5-yl)-1,2-dihydroquinolin-4-yl)amino)cyclohexanecarboxamide; 1-Methyl-4-((tetrahydro-2H-pyran-4-yl)amino)-6-(thiazol-5-yl)quinolin-2(1H)-one; 4-(((1r,4r)-4-(2-Methoxyethoxy)cyclohexyl)amino)-1-methyl-6-(thiazol-5-yl)quinolin-2(1H)-one; 4-(((1r,4r)-4-Methoxycyclohexyl)amino)-1-methyl-6-(thiazol-5-yl)quinolin-2(1H)-one; 4-(((1s,4s)-4-Hydroxycyclohexyl)amino)-1-methyl-6-(thiazol-5-yl)quinolin-2(1H)-one trifluoroacetic acid salt; 4-Amino-1-methyl-6-(thiazol-5-yl)quinolin-2(1H)-one; (1r,4r)-4-((1,8-Dimethyl-2-oxo-6-(thiazol-5-yl)-1,2-dihydroquinolin-4-yl)amino)-Nmethylcyclohexanecarboxamide; 1,8-Dimethyl-4-((tetrahydro-2H-pyran-4-yl)amino)-6-(thiazol-5-yl)quinolin-2(1H)-one; 4-(((1r,4r)-4-(2-Methoxyethoxy)cyclohexyl)amino)-1,8-dimethyl-6-(thiazol-5-yl)quinolin-2(1H)-one; 4-(((1r,4r)-4-Methoxycyclohexyl)amino)-1,8-dimethyl-6-(thiazol-5-yl)quinolin-2(1H)-one; (1r,4r)-4-((1-Ethyl-2-oxo-6-(thiazol-5-yl)-1,2-dihydroquinolin-4-yl)amino)-Nmethylcyclohexanecarboxamide; 1-Ethyl-4-(((1r,4r)-4-(2-methoxyethoxy)cyclohexyl)amino)-6-(thiazol-5-yl)quinolin-2(1H)-one; and derivatives thereof.

In one embodiment, the small organic molecule according to the invention is selected in the group comprising or consisting of those described in Sepehri & Ghavami (2017. J Biomol Struct Dyn. 35(9): 1890-8), which is incorporated herein by reference.

In one embodiment, the small organic molecule according to the invention is selected in the group comprising or consisting of those described in Scully et al. (2017. ACS Med Chem Lett. 8(2): 196-200), which is incorporated herein by reference.

In one embodiment, the small organic molecule according to the invention is selected from

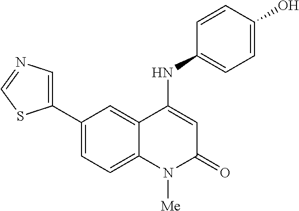

and derivatives thereof such as for example:

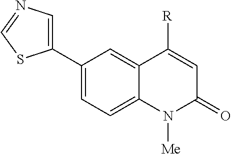

wherein R is selected from:

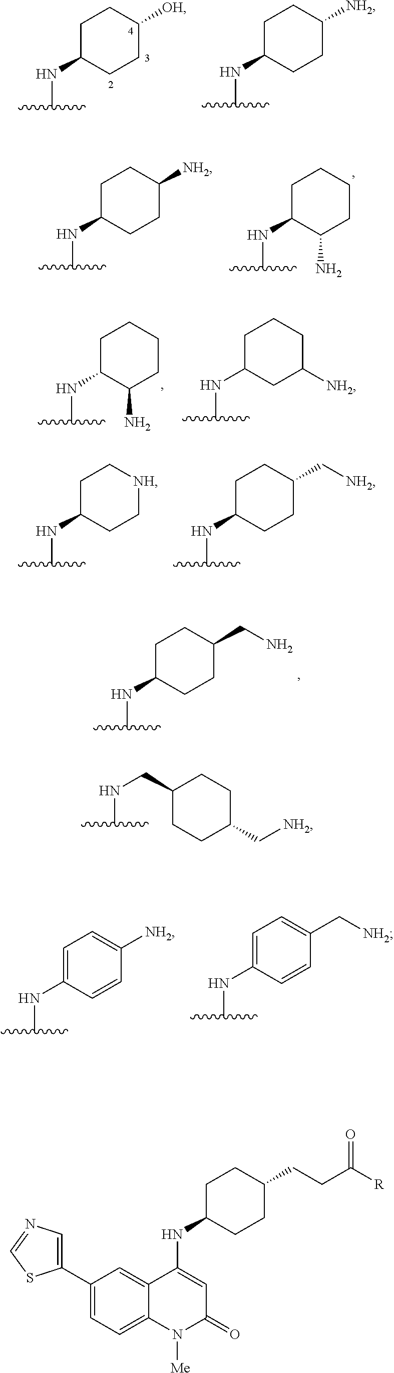

wherein R is selected from

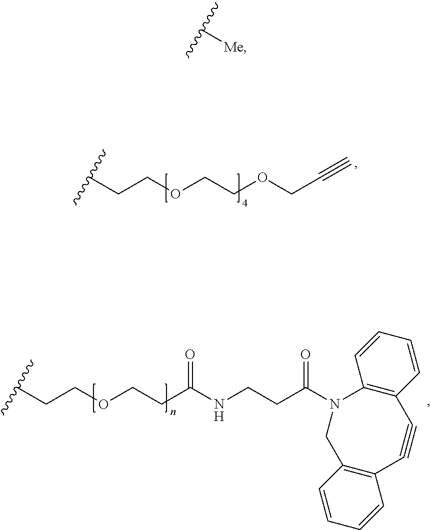

with n being either 5 or 13

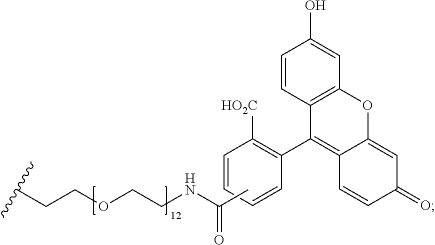

and derivatives thereof.

In one embodiment, the small organic molecule according to the invention is selected in the group comprising or consisting of those described in Deaton et al. (2018. Bioorg Med Chem. 26(8):2107-2150, which is incorporated herein by reference.

In one embodiment, the small organic molecule according to the invention is selected from

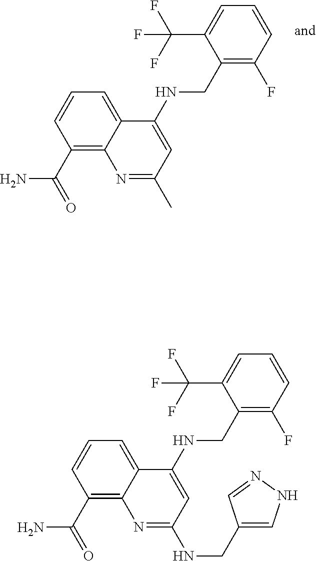 [SMILES: Cc1cc(NCc2c(F)cccc2C(F)(F)F)c2cccc(C(N)=O)c2n1 ; NC(=O)c1cccc2c(NCc3c(F)cccc3C(F)(F)F)cc(NCc3cn[nH]c3)nc12]

derivatives such as for example: 2-(((1H-Pyrazol-4-yl)methyl)amino)-4-((2-fluoro-6-(trifluoromethyl)benzyl)amino)quinazoline-8-carboxamide; 2-(((1H-Pyrazol-5-yl)methyl)amino)-4-((2-fluoro-6-(trifluoromethyl)benzyl)amino)quinazoline-8-carboxamide; 4-((2-Fluoro-6-(trifluoromethyl)benzyl)amino)-2-(((3-methyl-1H-pyrazol-4-yl)methyl)amino)quinazoline-8-carboxamide; 2-(4,6-Dihydropyrrolo[3,4-c]pyrazol-5(1H)-yl)-4-({[2-fluoro-6-(trifluoromethyl)phenyl]methyl}amino)-8-quinazolinecarboxamide; 2-(6,7-Dihydro-1H-pyrazolo[4,3-c]pyridin-5(4H)-yl)-4-((2-fluoro-6-(trifluoromethyl)benzyl)amino)quinazoline-8-carboxamide; 2-(4,5-Dihydro-1H-pyrazolo[3,4-c]pyridin-6(7H)-yl)-4-((2-fluoro-6-(trifluoromethyl)benzyl)amino)quinazoline-8-carboxamide; 2-(5,6-Dihydropyrazolo[3,4-c]azepin-7(1H,4H,8H)-yl)-4-((2-fluoro-6-(trifluoromethyl)benzyl)amino)quinazoline-8-carboxamide; (R)-4-((2-Fluoro-6-(trifluoromethyl)benzyl)amino)-2-(6-methylpyrrolo[3,4-c]pyrazol-5(1H,4H,6H)-yl)quinazoline-8-carboxamide; (S)-4-((2-fluoro-6-(trifluoromethyl)benzyl)amino)-2-(6-methylpyrrolo[3,4-c]pyrazol-5(1H,4H,6H)-yl)quinazoline-8-carboxamide; 4-((2-Fluoro-6-(trifluoromethyl)benzyl)amino)-2-(1-methylpyrrolo[3,4-c]pyrazol-5(1H,4H,6H)-yl)quinazoline-8-carboxamide; 4-((2-Fluoro-6-(trifluoromethyl)benzyl)amino)-2-(2-methylpyrrolo[3,4-c]pyrazol-5(2H,4H,6H)-yl)quinazoline-8-carboxamide; 4-((2-Fluoro-6-(trifluoromethyl)benzyl)amino)-2-(3-methylpyrrolo[3,4-c]pyrazol-5(1H,4H,6H)-yl)quinazoline-8-carboxamide; (S)-4-((2-Fluoro-6-(trifluoromethyl)benzyl)amino)-2-(4-methylpyrrolo[3,4-c]pyrazol-5(1H,4H,6H)-yl)quinazoline-8-carboxamide; (R)-4-((2-Fluoro-6-(trifluoromethyl)benzyl)amino)-2-(4-methylpyrrolo[3,4-c]pyrazol-5(1H,4H,6H)-yl)quinazoline-8-carboxamide; (R)-2-(6-Ethylpyrrolo[3,4-c]pyrazol-5(1H,4H,6H)-yl)-4-((2-fluoro-6-(trifluoromethyl)benzyl)amino)quinazoline-8-carboxamide; (S)-2-(6-ethylpyrrolo[3,4-c]pyrazol-5(1H,4H,6H)-yl)-4-((2-fluoro-6-(trifluoromethyl)benzyl)amino)quinazoline-8-carboxamide; (R)-2-(6-Ethylpyrrolo[3,4-c]pyrazol-5(1H,4H,6H)-yl)-4-((2-fluoro-6-(trifluoromethyl)benzyl)amino)quinazoline-8-carboxamide; (S)-2-(6-ethylpyrrolo[3,4-c]pyrazol-5(1H,4H,6H)-yl)-4-((2-fluoro-6-(trifluoromethyl)benzyl)amino)quinazoline-8-carboxamide; 2-(3-Ethylpyrrolo[3,4-c]pyrazol-5(1H,4H,6H)-yl)-4-((2-fluoro-6-(trifluoromethyl)benzyl)amino)quinazoline-8-carboxamide; 4-((2-Fluoro-6-(trifluoromethyl)benzyl)amino)-2-(3-(trifluoromethyl)pyrrolo[3,4-c]pyrazol-5(1H,4H,6H)-yl)quinazoline-8-carboxamide; 4-((2-Fluoro-6-(trifluoromethyl)benzyl)amino)-2-(3-oxo-2,3-dihydropyrrolo[3,4-c]pyrazol-5(1H,4H,6H)-yl)quinazoline-8-carboxamide trifluoroacetic acid salt; 2-(3-Aminopyrrolo[3,4-c]pyrazol-5(1H,4H,6H)-yl)-4-((2-fluoro-6-(trifluoromethyl)benzyl)amino)quinazoline-8-carboxamide; 2-(3-Carbamoylpyrrolo[3,4-c]pyrazol-5(1H,4H,6H)-yl)-4-((2-fluoro-6-(trifluoromethyl)benzyl)amino)quinazoline-8-carboxamide; (S)-4-((2-Fluoro-6-(trifluoromethyl)benzyl)amino)-2-(3-(1-hydroxyethyl)pyrrolo[3,4-c]pyrazol-5(1H,4H,6H)-yl)quinazoline-8-carboxamide trifluoroacetate; (R)-4-((2-fluoro-6-(trifluoromethyl)benzyl)amino)-2-(3-(1-hydroxyethyl)pyrrolo[3,4-c]pyrazol-5(1H,4H,6H)-yl)quinazoline-8-carboxamide trifluoroacetate; (S)-4-((2-Fluoro-6-(trifluoromethyl)benzyl)amino)-2-(3-(1-hydroxyethyl)pyrrolo[3,4-c]pyrazol-5(1H,4H,6H)-yl)quinazoline-8-carboxamide trifluoroacetate; (R)-4-((2-Fluoro-6-(trifluoromethyl)benzyl)amino)-2-(3-(1-hydroxyethyl)pyrrolo[3,4-c]pyrazol-5(1H,4H,6H)-yl)quinazoline-8-carboxamide trifluoroacetate; (R)-2-(3,6-Dimethylpyrrolo[3,4-c]pyrazol-5(1H,4H,6H)-yl)-4-((2-fluoro-6-(trifluoromethyl)benzyl)amino)quinazoline-8-carboxamide; (S)-2-(3,6-Dimethylpyrrolo[3,4-c]pyrazol-5(1H,4H,6H)-yl)-4-((2-fluoro-6-(trifluoromethyl)benzyl)amino)quinazoline-8-carboxamide; (R)-2-(3-Amino-6-methylpyrrolo[3,4-c]pyrazol-5(1H,4H,6H)-yl)-4-((2-fluoro-6-(trifluoromethyl)benzyl)amino)quinazoline-8-carboxamide; (S)-2-(3-Amino-6-methylpyrrolo[3,4-c]pyrazol-5(1H,4H,6H)-yl)-4-((2-fluoro-6-(trifluoromethyl)benzyl)amino)quinazoline-8-carboxamide; (R)-2-(3-Amino-6-methylpyrrolo[3,4-c]pyrazol-5(1H,4H,6H)-yl)-4-((2-fluoro-6-(trifluoromethyl)benzyl)amino)quinazoline-8-carboxamide; 2-(3-Amino-6,6-dimethylpyrrolo[3,4-c]pyrazol-5(1H,4H,6H)-yl)-4-((2-fluoro-6-(trifluoromethyl)benzyl)amino)quinazoline-8-carboxamide; 2-(30-Amino-10H-spiro[cyclopropane-1,60-pyrrolo[3,4-c]pyrazol]-50(40H)-yl)-4-((2-fluoro-6-(trifluoromethyl)benzyl)amino)quinazoline-8-carboxamide; 2-(30-Amino-10H-spiro[cyclobutane-1,60-pyrrolo[3,4-c]pyrazol]-50(40H)-yl)-4-((2-fluoro-6-(trifluoromethyl)benzyl)amino)quinazoline-8-carboxamide; 2-(30-Amino-10H-spiro[cyclopentane-1,60-pyrrolo[3,4-c]pyrazol]-50(40H)-yl)-4-((2-fluoro-6-(trifluoromethyl)benzyl)amino)quinazoline-8-carboxamide; and derivatives thereof.

In one embodiment, the small organic molecule according to the invention inhibits the NAADP hydrolase activity of CD38 (particularly of human CD38) with an IC₅₀ inferior or equal to 5 μM, in particular inferior or equal to 500 nM, more particularly inferior or equal to 50 nM; or activates the NAADP synthase activity of CD38 (particularly of human CD38) with an EC₅₀ inferior or equal to 5 μM, in particular inferior or equal to 500 nM, more particularly inferior or equal to 50 nM.

In one embodiment, the small organic molecule according to the invention inhibits the ability of CD38, in particular human CD38, to degrade NAADP with an IC₅₀ inferior or equal to 5 μM, in particular inferior or equal to 500 nM, more particularly inferior or equal to 50 nM; or activates the ability of CD38, in particular human CD38, to synthetize NAADP with an EC₅₀ inferior or equal to 5 μM, in particular inferior or equal to 500 nM, more particularly inferior or equal to 50 nM.

Methods for determining the IC₅₀ of a small organic molecule according to the invention that inhibits the ability of CD38, in particular human CD38, to degrade NAADP can be assayed in vitro by using a solution comprising at least NAADP, CD38 (from recombinant CD38 proteins, extracted cellular proteins or whole cells expressing CD38) in the presence or not of the compounds of interest by following NAADP degradation by HPLC as described in Schmid et al. (2011. FEBS Lett. 585(22):3544-8) or by another detection method like ELISA, or by following ADPRP formation.

Methods for determining the EC₅₀ of a small organic molecule according to the invention that activate the ability of CD38, in particular human CD38, to synthetize NAADP can be assayed in vitro by using a solution comprising at least NADP, nicotinic acid, CD38 (from recombinant CD38 proteins, extracted cellular proteins or whole cells expressing CD38) in the presence or not of the compounds of interest by following NAADP synthesis by HPLC as described in Schmid et al. (2011. FEBS Lett. 585(22):3544-8) or by another detection method like ELISA, or by following NADP degradation.

In one embodiment, the small organic molecule according to the invention specifically binds to at least one, in particular at least two, more particularly at least three amino-acid(s) of human CD38 with SEQ ID NO: 1 selected from the group comprising or consisting of glutamic acid 146, aspartic acid 155 and glutamic acid 226 (Graeff et al., 2006. J Biol Chem. 281(39):28951-7).

In particular, the small organic molecule according to the invention specifically binds to glutamic acid 146, aspartic acid 155 and glutamic acid 226 of human CD38 with SEQ ID NO: 1.

As used herein, the term “neurodegenerative disease” refers to any condition susceptible of being improved or prevented by the protection and/or prevention of neurodegeneration.

In particular, the neurodegenerative disease is selected in the group comprising or consisting of Parkinson's disease and related disorders (including, but not limited to, Parkinson's disease, Parkinson-dementia, autosomal recessive PARK2 and PARK6-linked Parkinsonism, atypical parkinsonian syndromes [such as, e.g., progressive supranuclear palsy, corticobasal degeneration syndrome, Lewy bodies dementia, multiple system atrophy], Guadeloupean Parkinsonism and Lytigo-bodig disease); motor neuron diseases (including, but not limited to, amyotrophic lateral sclerosis, frontotemporal dementia, progressive bulbar palsy, pseudobulbar palsy, primary lateral sclerosis, progressive muscular atrophy, spinal muscular atrophy and post-polio syndrome); neuro-inflammatory diseases; Alzheimer's disease and related disorders (including, but not limited to, early stage of an Alzheimer's disorder, mild stage of an Alzheimer's disorder, moderate stage of an Alzheimer's disorder, mild to moderate stage of an Alzheimer's disorder, advanced stage of an Alzheimer's disorder, mild cognitive impairment, vascular dementia, mixed dementia, Pick's disease, argyrophilic grain disease, posterior cortical atrophy, Wernicke-Korsakoff Syndrome); prion diseases (including, but not limited to, Creutzfeld-Jacob disease); lysosomal storage diseases; leukodystrophies; Huntington's disease; Down syndrome; spinal and bulbar muscular atrophy; stroke; traumatic brain injuries; HIV-associated neurocognitive disorder; Tourette syndrome; autosomal dominant spinocerebellar ataxia; multiple sclerosis; Friedreich's ataxia; Charcot-Marie-Tooth disease; dentatorubral pallidoluysian atrophy; myotonic dystrophy; schizophrenia; age-associated memory impairment; autism and autism spectrum disorders; attention-deficit hyperactivity disorder; chronic pain; alcohol-induced dementia; progressive non-fluent aphasia; semantic dementia; spastic paraplegia; fibromyalgia; post-Lyme disease; neuropathies; withdrawal symptoms; Alpers' disease; cerebro-oculo-facio-skeletal syndrome; Wilson's disease; Cockayne syndrome; Leigh's disease; neurodegeneration with brain iron accumulation; opsoclonus myoclonus syndrome; alpha-methylacyl-CoA racemase deficiency; Andermann syndrome; Arts syndrome; Marinesco-Sjögren syndrome; mitochondrial membrane protein-associated neurodegeneration; pantothenate kinase-associated neurodegeneration; polycystic lipomembranous osteodysplasia with sclerosing leukoencephalopathy; riboflavin transporter deficiency neuronopathy; and ataxia telangiectasia.

More particularly, the neurodegenerative disease is selected in the group comprising or consisting of Parkinson's disease; Lewy body dementia; multiple system atrophy; Alzheimer's disease; progressive supranuclear palsy; corticobasal degeneration; Pick's disease; frontotemporal dementia; amyotrophic lateral sclerosis; spinal and bulbar muscular atrophy; stroke; traumatic brain injuries; Huntington's disease; multiple sclerosis; Friedreich's ataxia; Charcot-Marie-Tooth disease; Creutzfeld-Jacob disease and other prion diseases; leukodistrophies; and lysosomal storage disorders.

As used herein, the term “inflammatory diseases” refers to diseases characterized by abnormal inflammation, such as in the case of autoimmune diseases or allergies, or an excess inflammation of tissues leading to chronic pain, redness, swelling, stiffness and/or damage to healthy tissues.

In particular, the inflammatory disease is selected in the group comprising or consisting of neuroinflammatory diseases (including, but not limited to, meningitis, encephalitis and autoimmune neuroinflammatory diseases [multiple sclerosis, Guillain-Barré syndrome, Baló disease, chronic inflammatory demyelinating polyneuropathy, Devic's disease, multifocal motor neuropathy, narcolepsy, neuromyelitis optica, optic neuritis, paraneoplastic cerebellar degeneration and transverse myelitis]); Gaucher's disease; autoimmune diseases (including, but not limited to, achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease, autoimmune myocarditis, autoimmune oophoritis, autoimmune orchitis, autoimmune pancreatitis, autoimmune retinopathy, Baló disease, Behcet's disease, benign mucosal pemphigoid, bullous pemphigoid, Castleman disease, Chagas disease, chronic inflammatory demyelinating polyneuropathy, chronic recurrent multifocal osteomyelitis, Churg-Strauss syndrome, cicatricial pemphigoid, coeliac disease, Cogan's syndrome, cold agglutinin disease, congenital heart block, Coxsackie myocarditis, CREST syndrome, dermatitis herpetiformis, Devic's disease, discoid lupus, Dressler's syndrome, endometriosis, eosinophilic esophagitis, eosinophilic fasciitis, erythema nodosum, essential mixed cryoglobulinemia, Evans syndrome, fibromyalgia, fibrosing alveolitis, giant cell arteritis, giant cell myocarditis, glomerulonephritis, Goodpasture's syndrome, granulomatosis with polyangiitis, Grave's disease, Guillain-Barré syndrome, haemolytic anaemia, Hashimoto's disease, Henoch-Schonlein purpura, herpes gestationis, hidradenitis suppurativa, hypogammaglobulinemia, idiopathic thrombocytopenic purpura, IgA nephropathy, IgG4-related sclerosing disease, immune thrombocytopenic purpura, inclusion body myositis, inflammatory bowel diseases, inflammatory myopathies, interstitial cystitis, juvenile arthritis, juvenile myositis, Kawasaki disease, Lambert-Eaton syndrome, leukocytoclastic vasculitis, lichen planus, lichen sclerosus, ligneous conjunctivitis, linear IgA disease, lupus, Lyme disease chronic, Meniere's disease, microscopic polyangiitis, mixed connective tissue disease, Mooren's ulcer, Mucha-Habermann disease, multifocal motor neuropathy, multiple sclerosis, myasthenia gravis, myositis, narcolepsy, neonatal lupus, neuromyelitis optica, neutropenia, ocular cicatricial pemphigoid, optic neuritis, palindromic rheumatism, paraneoplastic cerebellar degeneration, paroxysmal nocturnal hemoglobinuria, Parry Romberg syndrome, pars planitis, Parsonnage-Turner syndrome, pediatric autoimmune neuropsychiatric disorders associated with streptococcal infections (PANDAS), pemphigus, peripheral neuropathy, perivenous encephalomyelitis, pernicious anemia, POEMS syndrome, polyarteritis nodosa, polyglandular syndromes, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome, subacute bacterial endocarditis, Sucac's syndrome sympathetic ophtalmia, systemic lupus erythematosus, Takayasu's arteritis, temporal arteritis, Tolosa-Hunt syndrome, transverse myelitis, type 1 diabetes, undifferentiated connective tissue disease, uveitis, vasculitis Vogt-Koyanagi-Harada disease, vitiligo and Wegener's granulomatosis); allergy; asthma; hepatitis (including, but not limited to, acute hepatitis, fulminant hepatitis, chronic hepatitis, viral hepatitis, parasitic hepatitis, bacterial hepatitis, alcoholic hepatitis, toxic and drug-induced hepatitis, steatohepatitis, alpha-1-antitrypsin deficiency, hemochromatosis, Wilson's disease ischemic hepatitis, nonalcoholic & alcoholic steatohepatitis); reperfusion injury; type 2 diabetes; and transplant rejection.

More particularly, the inflammatory disease is selected in the group comprising or consisting of neuroinflammatory diseases, including, but not limited to, meningitis, encephalitis, multiple sclerosis, Guillain-Barré syndrome, Baló disease, chronic inflammatory demyelinating polyneuropathy, Devic's disease, multifocal motor neuropathy, narcolepsy, neuromyelitis optica, optic neuritis, paraneoplastic cerebellar degeneration and transverse myelitis.

More particularly, the inflammatory disease is selected in the group comprising or consisting of autoimmune diseases, including, but not limited to, achalasia, Addison's disease, adult Still's disease, agammaglobulinemia, alopecia areata, amyloidosis, ankylosing spondylitis, anti-GBM/anti-TBM nephritis, antiphospholipid syndrome, autoimmune angioedema, autoimmune dysautonomia, autoimmune encephalomyelitis, autoimmune hepatitis, autoimmune inner ear disease, autoimmune myocarditis, autoimmune oophoritis, autoimmune orchitis, autoimmune pancreatitis, autoimmune retinopathy, Baló disease, Behcet's disease, benign mucosal pemphigoid, bullous pemphigoid, Castleman disease, Chagas disease, chronic inflammatory demyelinating polyneuropathy, chronic recurrent multifocal osteomyelitis, Churg-Strauss syndrome, cicatricial pemphigoid, coeliac disease, Cogan's syndrome, cold agglutinin disease, congenital heart block, Coxsackie myocarditis, CREST syndrome, dermatitis herpetiformis, Devic's disease, discoid lupus, Dressler's syndrome, endometriosis, eosinophilic esophagitis, eosinophilic fasciitis, erythema nodosum, essential mixed cryoglobulinemia, Evans syndrome, fibromyalgia, fibrosing alveolitis, giant cell arteritis, giant cell myocarditis, glomerulonephritis, Goodpasture's syndrome, granulomatosis with polyangiitis, Grave's disease, Guillain-Barre syndrome, haemolytic anaemia, Hashimoto's disease, Henoch-Schonlein purpura, herpes gestationis, hidradenitis suppurativa, hypogammaglobulinemia, idiopathic thrombocytopenic purpura, IgA nephropathy, IgG4-related sclerosing disease, immune thrombocytopenic purpura, inclusion body myositis, inflammatory bowel diseases, inflammatory myopathies, interstitial cystitis, juvenile arthritis, juvenile myositis, Kawasaki disease, Lambert-Eaton syndrome, leukocytoclastic vasculitis, lichen planus, lichen sclerosus, ligneous conjunctivitis, linear IgA disease, lupus, Lyme disease chronic, Meniere's disease, microscopic polyangiitis, mixed connective tissue disease, Mooren's ulcer, Mucha-Habermann disease, multifocal motor neuropathy, multiple sclerosis, myasthenia gravis, myositis, narcolepsy, neonatal lupus, neuromyelitis optica, neutropenia, ocular cicatricial pemphigoid, optic neuritis, palindromic rheumatism, paraneoplastic cerebellar degeneration, paroxysmal nocturnal hemoglobinuria, Parry Romberg syndrome, pars planitis, Parsonnage-Turner syndrome, pediatric autoimmune neuropsychiatric disorders associated with streptococcal infections (PANDAS), pemphigus, peripheral neuropathy, perivenous encephalomyelitis, pernicious anemia, POEMS syndrome, polyarteritis nodosa, polyglandular syndromes, polymyalgia rheumatica, polymyositis, postmyocardial infarction syndrome, postpericardiotomy syndrome, primary biliary cirrhosis, primary sclerosing cholangitis, progesterone dermatitis, psoriasis, psoriatic arthritis, pure red cell aplasia, pyoderma gangrenosum, Raynaud's phenomenon, reactive arthritis, reflex sympathetic dystrophy, relapsing polychondritis, restless legs syndrome, retroperitoneal fibrosis, rheumatic fever, rheumatoid arthritis, sarcoidosis, Schmidt syndrome, scleritis, scleroderma, Sjögren's syndrome, sperm and testicular autoimmunity, stiff person syndrome, subacute bacterial endocarditis, Sucac's syndrome sympathetic ophtalmia, systemic lupus erythematosus, Takayasu's arteritis, temporal arteritis, Tolosa-Hunt syndrome, transverse myelitis, type 1 diabetes, undifferentiated connective tissue disease, uveitis, vasculitis Vogt-Koyanagi-Harada disease, vitiligo and Wegener's granulomatosis.

The invention also relates to a compound according to the invention, for use as a medicament, wherein said compound is administered to a non-responder patient to a treatment targeting the NADtdependent pathway and/or the cADPR-dependent pathway.

As used herein, a “medicament” is meant to encompass a composition suitable for administration to a subject or patient, such as a mammal, especially a human. In general, a “medicament” is sterile and is usually free of contaminants that are capable of eliciting an undesirable response within the subject (e.g., the compound(s) in the medicament is pharmaceutical grade). Medicaments can be designed for administration to subjects in need thereof via a number of different routes of administration including oral, buccal, rectal, parenteral, intraperitoneal, intradermal, subcutaneous, intranasal, intrathecal, perispinal and the like. The preferred form depends on the intended mode of administration and therapeutic application. Typical preferred form are oral, injectable or infusible solutions.

The present invention also relates to a composition comprising or consisting of or consisting essentially of the compound according to the present invention.

The present invention also relates to a pharmaceutical composition comprising or consisting of or consisting essentially of the compound according to the present invention and at least one pharmaceutically acceptable carrier.

As used herein, a “pharmaceutically acceptable carrier” is meant to encompass an excipient, diluent, carrier, and adjuvant that are useful in preparing a pharmaceutical composition that are generally safe, non-toxic and neither biologically nor otherwise undesirable, and include an excipient, diluent, carrier, and adjuvant that are acceptable for veterinary use as well as human pharmaceutical use. A “pharmaceutically acceptable carrier” as used herein includes both one and more than one such excipient, diluent, carrier, and adjuvant.

The present invention also relates to a medicament comprising or consisting of or consisting essentially of the compound according to the present invention.

As used herein, the term “consisting essentially of”, with reference to a composition, pharmaceutical composition or medicament, means that the compound according to the invention is the only one agent with a biologic activity within said composition, pharmaceutical composition or medicament.

In particular, the composition, pharmaceutical composition or medicament according to the present invention comprises or consists of, as an active ingredient, a compound according to the invention, supplemented with a pharmaceutically acceptable carrier.

The invention also relates to the use of a compound, which specifically binds to CD38 and activates the opening of NAADP receptors Two Pore Channels TPC1 and/or TPC2 for the preparation of a medicament for the prevention and/or treatment of a neurodegenerative and/or inflammatory disease. Such compounds (antibody or antigen-binding fragment thereof or antigen-binding antibody mimetic or small organic molecule according to the invention) can be present in the medicament according to the invention in a therapeutically amount (active and non-toxic amount).

The invention also relates to a method of treatment of a neurodegenerative and/or inflammatory disease comprising the administration of a compound, which specifically binds to CD38 and activates the opening of NAADP receptors Two Pore Channels TPC1 and/or TPC2 according to the invention in a therapeutically amount to a subject in need thereof.

The term “therapeutically effective amount” refers to level or amount of agent that is aimed at, without causing significant negative or adverse side effects to the target, (1) delaying or preventing the onset of a neurodegenerative and/or inflammatory disease; (2) slowing down or stopping the progression, aggravation, or deterioration of one or more symptoms of a neurodegenerative and/or inflammatory disease; (3) bringing about ameliorations of the symptoms of a neurodegenerative and/or inflammatory disease; (4) reducing the severity or incidence of a neurodegenerative and/or inflammatory disease; or (5) curing a neurodegenerative and/or inflammatory disease.

Such therapeutically amount can be determined by one skilled in the art by routine tests including assessment of the effect of administration of said components on the pathologies and/or disorders which are sought to be prevented and/or to be treated by the administration of said pharmaceutical composition or medicament according to the invention.

For example, such tests can be implemented by analyzing both quantitative and qualitative effect of the administration of different amounts of said aforementioned compounds (antibody or antigen-binding fragment thereof or antigen-binding antibody mimetic or small organic molecules according to the invention) on a set of markers (biological and/or clinical) characteristics of said pathologies and/or of said disorders, in particular from a biological sample of a subject.

A therapeutically effective amount may be administered prior to the onset of a neurodegenerative and/or inflammatory disease, for a prophylactic or preventive action. Alternatively, or additionally, the therapeutically effective amount may be administered after the onset of a neurodegenerative and/or inflammatory disease, for a therapeutic action. In one embodiment, a therapeutically effective amount of the composition is an amount that is effective in reducing at least one symptom of a neurodegenerative and/or inflammatory disease.

In one embodiment, the composition, pharmaceutical composition or medicament according to the present invention is to be administered using an intrathecal route of administration, i.e., intrathecally.

The efficient delivery using an intrathecal route of administration of a compound similar in size to the compound of the present invention, such as for example Rituximab, has been previously exemplified in clinical trial NCT01719159, or Nivolumab, has been previously exemplified in clinical trial NCT03025256.

Sterile medicaments for intrathecal administration can be prepared by incorporating the compound of the present invention in the required amount in the appropriate solvent, followed by sterilization by microfiltration. As solvent or vehicle, there may be used water, saline, phosphate buffered saline, dextrose, glycerol, ethanol, and the like, as well as combination thereof. In many cases, it will be preferable to include isotonic agents, such as sugars, polyalcohols, or sodium chloride in the composition. These medicaments may also contain adjuvants, in particular wetting, isotonizing, emulsifying, dispersing and stabilizing agents.

In one embodiment, the composition, pharmaceutical composition or medicament according to the present invention is to be administered using a subcutaneous route of administration, i.e., subcutaneously.

The efficient delivery using a subcutaneous route of administration of a compound similar in size to the compound of the present invention, such as for example Daclizumab, has been previously exemplified in clinical trial NCT00109161.

In a preferred embodiment, the composition, pharmaceutical composition or medicament according to the present invention is to be administered using an intravenous route of administration, i.e., intravenously.

The efficient delivery using an intravenous route of administration of a compound similar in size to the compound of the present invention, such as for example Aducanumab, has been previously exemplified in clinical trial NCT01677572 and in Sevigny et al. (2016. Nature. 537(7618):50-6).

Sterile medicaments for parenteral administration may also be prepared in the form of sterile solid compositions which may be dissolved at the time of use in sterile water or any other injectable sterile medium.

In one embodiment, the composition, pharmaceutical composition or medicament according to the present invention is to be orally administered. As solid medicaments for oral administration, tablets, pills, powders (gelatine capsules, sachets) or granules may be used. In these medicaments, the active ingredient according to the invention is mixed with one or more inert diluents, such as starch, cellulose, sucrose, lactose or silica, under an argon stream. These medicaments may also comprise substances other than diluents, for example one or more lubricants such as magnesium stearate or talc, a coloring, a coating (sugar-coated tablet) or a glaze. As liquid medicaments for oral administration, there may be used pharmaceutically acceptable solutions, suspensions, emulsions, syrups and elixirs containing inert diluents such as water, ethanol, glycerol, vegetable oils or paraffin oil. These medicaments may comprise substances other than diluents, for example wetting, sweetening, thickening, flavoring or stabilizing products.

In one embodiment, the compound, the composition, pharmaceutical composition or medicament of the invention is to be administered at a dose determined by the skilled artisan and personally adapted to each subject.

It will be understood that the total daily usage of the compound, the composition, pharmaceutical composition or medicament of the invention will be decided by the attending physician within the scope of sound medical judgment. The specific therapeutically or nutraceutically effective amount for any particular subject will depend upon a variety of factors including the disease being treated and the severity of the disease; the specific composition employed, the age, body weight, general health, sex and diet of the subject; the time of administration, route of administration, the duration of the treatment; drugs used in combination or coincidental with the compound, the composition, pharmaceutical composition or medicament of the invention; and like factors well-known in the medical arts. For example, it is well within the skill of the art to start doses of a therapeutic compound at levels lower than those required to achieve the desired therapeutic effect and to gradually increase the dosage until the desired effect is achieved; but, at the opposite, it can be equally useful to start with a loading dose, a manner to reach steady-state plasma concentration more quickly, and then to follow with a maintenance dose calculated to exactly compensate the effect of the elimination process.

In one embodiment, a therapeutically effective amount of the compound, the composition, pharmaceutical composition or medicament of the invention is to be administered at least once a day, at least twice a day, at least three times a day.

In one embodiment, a therapeutically effective amount of the compound, the composition, pharmaceutical composition or medicament of the invention is to be administered every two, three, four, five, six days.

In one embodiment, a therapeutically effective amount of the compound, the composition, pharmaceutical composition or medicament of the invention is to be administered twice a week, every week, every two weeks, once a month.

In one embodiment, a therapeutically effective amount of the compound, the composition, pharmaceutical composition or medicament of the invention is to be administered every month, every two months, every three months, every four months, every five months, every six months, once a year.

In one embodiment, a therapeutically effective amount of the compound, the composition, pharmaceutical composition or medicament of the invention is to be administered for a period of time of about one day, two days, three days, four days, five days, six days, a week, two weeks, three weeks, a month, two months, three months, six months, a year, or over longer periods such as, e.g., for several years or for the rest of the life of the subject. In one embodiment, a therapeutically effective amount of the compound, the composition, pharmaceutical composition or medicament of the invention is to be administered until treatment or alleviation of the neurodegenerative and/or inflammatory disease.

In one embodiment, a therapeutically effective amount of the compound, the composition, pharmaceutical composition or medicament of the invention is to be administered for a chronic treatment. In one embodiment, a therapeutically effective amount of the compound, the composition, pharmaceutical composition or medicament of the invention is to be administered for an acute treatment.

In one embodiment, a therapeutically effective amount of the compound, the composition, pharmaceutical composition or medicament of the invention, preferably when administered using an intrathecal route of administration, is ranging from about 0.1 mg/kg to about 10 mg/kg, preferably is ranging from about 0.1 mg/kg to 5 mg/kg and more preferably is about 1 mg/kg.

In one embodiment, a therapeutically effective amount of the compound, the composition, pharmaceutical composition or medicament of the invention, preferably when administered using an intravenous route of administration, is ranging from about 0.1 mg/kg to about 250 mg/kg, preferably is ranging from about 1 mg/kg to about 200 mg/kg, from about 2.5 mg/kg to 100 mg/kg, from about 5 mg/kg to about 100 mg/kg, more preferably is ranging from about 10 mg/kg to about 30 mg/kg, from about 15 mg/kg to about 20 mg/kg and even more preferably is about 15 mg/kg.

In one embodiment, a therapeutically effective amount of the compound, the composition, pharmaceutical composition or medicament of the invention, preferably when administered using a subcutaneous route of administration, is ranging from about 1 mg to 1000 mg, preferably is ranging from about 10 to 250 mg, and more preferably is about 100 mg.

In one embodiment, the compound of the invention is able to cross the blood-brain barrier (BBB), preferably when using an intravenous or subcutaneous route of administration.

Methods to deliver antibodies injected intravenously across the BBB are known in the art. Indeed, several publications demonstrated that intravenous injection of monoclonal antibodies effectively crossed the blood-brain barrier (BBB). Pharmacokinetic modeling of brain concentration of intravenously injected monoclonal antibody demonstrated that around 0.4% of plasma mAb concentrations are found in the brain (Shah & Bets, 2013. MAbs. 5(2):297-305). Brain/plasma ratio of Aducanumab and ABBV-8E12 were found to reach 1.3% and 0.385% in humans, respectively (Sevigny et al. 2016. Nature. 537(7618):50-6; West et al., 2017. J Prev Alz Dis. 4(4):236-241). Moreover, several strategies have been developed to improve BBB crossing including the use of bispecific antibodies targeting insulin receptor or transferrin receptor (for review see Neves et al., 2016. Trends Biotech. 34(1):36-48) or using ultrasound-induced BBB opening, as described in Konofagou et al., 2012. Curr Pharm Biotechnol. 13(7):1332-45.

The doses depend on the desired effect, the duration of the treatment and the route of administration used; they are generally between 5 mg and 1000 mg per day orally for an adult with unit doses ranging from 0.01 mg to 250 mg of active substance. In general, the doctor will determine the appropriate dosage depending on the age, weight and any other factors specific to the subject to be treated.

The invention also relates to a combination product comprising as active ingredients:
at least one compound according to the invention; and
at least one second therapeutic agent selected from the group comprising or consisting of neuroprotective agents, symptomatic agents, probiotics and antibodies used to neutralize aggregated or aggregation-prone proteins;
   for its use as a medicament in the prevention and/or treatment of a neurodegenerative and/or inflammatory disease, by the opening of NAADP receptors Two Pore Channels TPC1 and/or TPC2, wherein said active ingredients are formulated for separate, simultaneous, or sequential administration.

The invention also relates to a combination product comprising as active ingredients:
at least one compound according to the invention; and
at least one second therapeutic agent selected from the group comprising or consisting of neuroprotective agents, symptomatic agents, probiotics, and antibodies used to neutralize aggregated or aggregation-prone proteins.
   for its separate, simultaneous, or sequential use as a medicament in the prevention and/or treatment of a neurodegenerative and/or inflammatory disease, by the opening of NAADP receptors Two Pore Channels TPC1 and/or TPC2.

As used herein, the term “neuroprotective agent” encompasses any agent having a neuroprotective effect.

As used herein the term “probiotic” refers to any substance which stimulates the growth of and/or contains microorganisms with beneficial properties, such as those of the intestinal flora. For example, said probiotic can contain the strains of S. thermophilus, B. animalis, L. bulgaricus, Lactococcus lactis, Lactobacillus acidophilus, Lactobacillus casei, Bifidobacterium bifidum, and Lactobacillus fermentum. It has been described that the regular intake of such probiotics resulted in an improvement of the cognitive capabilities.

As used herein the term “antibody used to neutralize aggregated or aggregation-prone proteins” refers to any antibody targeting α-synuclein, APP or any APP fragment (including Aβ₁₋₄₂), Tau, prion, or polyglutamine-extended proteins. For example, said antibody used to neutralize aggregated or aggregation-prone proteins can be solanezumab or aducanumab to neutralize Aβ peptides, armanezumab to neutralize Tau, as well as PRX002 or PD0805 to neutralize α-synuclein.

As used herein the term “symptomatic agent” refers to any agent used to improve the quality of life of patients suffering from neurodegenerative diseases without altering the progression of the pathology.

In particular, when the neurodegenerative or inflammatory disease is amyotrophic lateral sclerosis (ALS), said second therapeutic agent can be a neuroprotective agent selected in the group comprising or consisting of Riluzole, Edaravone (free radical scavenger) and masitinib.

In particular, when the neurodegenerative or inflammatory disease is an Alzheimer's disease, said second therapeutic agent can be a symptomatic agent selected in the group comprising or consisting of cholinesterase inhibitors (e.g., donepezil, galantamine, memantine, rivastigmine) and NMDA receptor antagonists (e.g., memantine).

In particular, when the neurodegenerative or inflammatory disease is a Parkinson's disease, said second therapeutic agent can be a symptomatic agent selected in the group comprising or consisting of dopamine mimetics (e.g., Carbidopa, Levodopa, association of both products), dopamine agonists (e.g., apomorphine, bromocriptine, rotigotine, pramipexole, Ropinirole), anticholinergic (e.g., benzatropine, trihexyphenidyl), MAO-B inhibitors (e.g., selegiline, rasagiline), COM-T inhibitors (e.g., entacapone, tolcapone) and amantadine, droxidopa, pimavanersin, rivastigmine.

In particular, when the neurodegenerative or inflammatory disease is a Huntington's disease, said second therapeutic agent can be a symptomatic agent selected in the group comprising or consisting of antipsychotic (e.g., haloperidol, chlorpromazine, risperidone, quetiapine, olanzapine), antidepressant (e.g., citalopram, fluoxetine, olanzapine), mood-stabilizing drugs (e.g., valproate, carbamazepine, lamotrigine) and tetrabenazine, amantadine, levetiracetam, clonazepam.

In particular, when the neurodegenerative or inflammatory disease is spinal muscular atrophy (SMA), said second therapeutic agent can be nusinersen.

In particular, when the neurodegenerative or inflammatory disease is a Friedreich's ataxia, said second therapeutic agent can be a neuroprotective agent selected in the group comprising or consisting of 5-hydroxytryptophan, coenzyme Q, Idebenone.

In particular, when the neurodegenerative or inflammatory disease is a multiple sclerosis, said second therapeutic agent can be an anti-inflammatory or neuroprotective agent selected in the group comprising or consisting of interferon beta-1a, peginterferon beta-1a, interferon beta-1b, glatiramer acetate, daclizumab, teriflunomide, fingolimod, dimethyl fumarate, alemtuzumab, mitoxantrone, ocrelizumab, natalizumab, prednisone, methylprednisolone, baclofen, tizanidine.

In particular, when the neurodegenerative or inflammatory disease is a stroke, said second therapeutic agent can be an anti-clotting agent selected in the group comprising or consisting of anticoagulants (e.g., warfarin), antiplatelet (e.g., aspirin, dipyridamole, clopidogrel), or agents as tissue plasminogen activator, alteplase, statins, angiotensin II receptor blockers, ACE inhibitors, beta-blockers, calcium channel blockers, diuretics.

In particular, when the neurodegenerative or inflammatory disease is a Gaucher's disease, said second therapeutic agent can be an enzyme replacement treatment or a substrate reduction therapy selected in the group comprising or consisting of imiglucerase, velaglucerase, eliglustat, miglustat.

The invention also relates to a method for increasing the level of at least one anti-inflammatory cytokine, in the blood of a subject, comprising administering a compound, which specifically binds to CD38 and activates the opening of NAADP receptors Two Pore Channel TPC1 and/or TPC2 according to the invention in a therapeutically effective amount to said subject in need thereof.

Examples of anti-inflammatory cytokines include but are not limited to, interleukin-10 (IL-10), transforming growth factor 13 (TGF-β), interleukin-1ra (IL-1ra), interleukin-4 (IL-4), interleukin-6 (IL-6), interleukin-11 (IL-11), interleukin-13 (IL-13) and interleukin-22 (IL-22).

Methods to measure blood cytokine level in a subject are known in the art. Such methods include, for example, the use of ELISA test, such as the one described in the Example section bellow.

The invention also relates to a method for increasing interleukin-10 (IL-10) level in the blood of a subject, comprising administering a compound, which specifically binds to CD38 and activates the opening of NAADP receptors Two Pore Channels TPC1 and/or TPC2 according to the invention in a therapeutically effective amount to said subject in need thereof.

Methods to measure IL-10 level in a blood sample from a subject are well-known in the art and include, e.g., the use of an ELISA test.

In one embodiment, the administration of a compound according to the invention increases IL-10 level in the blood of a subject, by at least 100%, 200%, 300%, particularly at least 400% and more particularly at least 500%, as compared to the administration of negative control molecule.

The invention also relates to a method of manufacturing a compound according to the present invention, which comprises the step of selecting a compound which specifically binds to CD38 and which activates the opening of NAADP receptors Two Pore Channels TPC1 and/or TPC2.

In particular, the step of selecting an antibody or an antigen-binding fragment thereof or an antigen-binding antibody mimetic which specifically binds to CD38 according to the invention comprising or consisting of selecting an antibody or an antigen-binding fragment thereof or an antigen-binding antibody mimetic which has a K_{D} value inferior or equal to 10⁻⁷ M, preferably inferior or equal to 10⁻⁸, more preferably inferior or equal to 10⁻⁹ M for CD38, even more preferably inferior or equal to 1.10⁻¹⁹ M, as may be determined by biosensor analysis, particularly by Biacore Analysis.

In particular, the step of selecting a small organic molecule which specifically binds to CD38 according to the invention comprising or consisting of selecting a small organic molecule which has a K_{D} value inferior or equal to 10⁻⁶ M, preferably inferior or equal to 10⁻⁷ M for CD38, more preferably inferior or equal to 1.10⁻⁸M, as may be determined by biosensor analysis, particularly by Biacore Analysis.

In particular, the step of selecting an oligonucleotide which specifically binds to CD38 according to the invention comprising or consisting of selecting a small organic molecule which has a K_{D} value inferior or equal to 200 nM, preferably inferior or equal to 150 nM for CD38, more preferably inferior or equal to 100 nM, as may be determined by biosensor analysis, particularly by Biacore Analysis.

As above indicated, the capacity of a compound to activate the opening of NAADP receptors Two Pore Channels TPC1 and/or TPC2 can be tested by using an inhibitor of TPC1 and/or TPC2 activation, such as NedK and Ned-19, as will be further described in the Examples section below.

In particular, the step of selecting a compound which activates the opening of NAADP receptors Two Pore Channels TPC1 and/or TPC2 comprising or consisting of selecting a compound, which neuroprotective effect is antagonized by at least 20%, 30%, 40%, 50%, particularly at least 60% and more particularly at least 70% in the presence of Ned-19, as compared to a negative control in the absence of Ned-19 in a neuroprotective assay as will be further described in the Examples section below. These neuroprotective assays include, but are not limited to, assays on prevention of spontaneous and progressive dopaminergic (DA) neuron death in midbrain cultures, protection of dopaminergic neurons against the mitochondrial neurotoxin MPP⁺ and protection of cortical neurons from excitotoxicity, in the presence or absence of Ned-19, as will be further described in the Examples section below.

The method of the invention can further comprise a step of selecting a compound which inhibits the NAADP hydrolase activity of CD38 or which activates the NAADP synthase activity of CD38.

The method of the invention can further comprise a step of selecting a compound which inhibits the ability of CD38 to degrade NAADP or which activates the ability of CD38 to synthetize NAADP.

The capacity of a compound to inhibit the NAADP hydrolase activity of CD38 (i.e., the ability of CD38 to degrade NAADP) or to activate the NAADP synthase activity of CD38 (i.e., the ability of CD38 to synthetize NAADP) can be tested using methods described in Schmid et al. (2011. FEBS Lett. 585(22):3544-8) as mentioned above.

The method of the invention can further comprise a step of selecting a compound which increases intracellular NAADP levels, in particular in neurons by inhibiting the ability of CD38 to degrade NAADP (i.e., by inhibiting the NAADP hydrolase activity of CD38) or by activating the ability of CD38 to synthetize NAADP (i.e., by activating the NAADP synthase activity of CD38), preferably by at least 10%, 20%, 30%, 40%, 50%, particularly at least 60% and more particularly at least 70%, as compared to a negative control molecule, in a NAADP level measurement assay.

The method of the invention can further comprise a step of selecting a compound which increase cytosolic calcium levels, in particular in neurons, preferably by at least 10%, 20%, particularly at least 30% and more particularly at least 40%, as compared to a negative control molecule, in an intracellular calcium level measurement assay.

The method of the invention can also comprise a step of selecting an antibody or antigen binding fragment thereof or an antigen-binding antibody mimetic which specifically binds to:
at least one peptide comprising amino acids 220 to 285 of human CD38 with SEQ ID NO: 1; and/or
to cysteine 254 and/or 275 of human CD38 with SEQ ID NO: 1; and/or
to the 5^{th} C-terminal disulfide loop involving cysteine 254 and cysteine 275 of human CD38 with SEQ ID NO: 1.

The method of the invention can also comprise a step of selecting a small organic molecule which inhibits the ability of CD38, in particular human CD38, to degrade NAADP with an IC₅₀ inferior or equal to 5 μM, in particular inferior or equal to 500 nM, more particularly inferior or equal to 50 nM or activates the ability of CD38, in particular human CD38, to synthetize NAADP with an EC₅₀ inferior or equal to 5 μM, in particular inferior or equal to 500 nM, more particularly inferior or equal to 50 nM.

The method of the invention can also comprise a step of selecting a small organic molecule which specifically binds to at least one, in particular at least two, more particularly the three amino-acid(s) of human CD38 with SEQ ID NO: 1 selected from the group comprising or consisting of glutamic acid 146, aspartic acid 155 and glutamic acid 226.

### BRIEF DESCRIPTION OF DRAWINGS

FIG. 1 is a bargraph showing the neuroprotective effect of NAD⁺ (1) and several anti-CD38 antibodies (HB7 (2), AT1 (3), clone 90 (4), AT13/5 (5) or OKT-10 (6)) in a model of midbrain cultures where these neurons degenerate spontaneously, selectively and progressively as they mature. Number of DA (TH⁺) neurons in 10-days in vitro (DIV) cultures chronically exposed to NAD⁺ (3 mM) or several anti-CD38 antibodies (clone 90, AT1, AT13/5, OKT-10 or HB-7; 1 μg/mL) in the presence or not of Ned-19 (2 μM), an inhibitor of NAADP receptor activation. Results are expressed in % of TH⁺ neurons in control cultures at DIV10. $ P<0.05 vs untreated cultures.

FIG. 2 is a bargraph showing the neuroprotective effect of NAD⁺ (1) and several anti-CD38 antibodies (HB7 (2), AT1 (3), clone 90 (4), AT13/5 (5) or OKT-10 (6)) for DA (TH+) neurons in a culture model where these neurons degenerate following GDNF withdrawal. DA cell survival in midbrain cultures initially exposed to 20 ng/mL GDNF for 10 DIV and then deprived of the peptide between 11 and 15 DIV exposed or not to NAD⁺ (3 mM) or several anti-CD38 antibodies (clone 90, AT1, AT13/5, OKT-10 or HB-7; 1 μg/mL) in the presence or not of Ned-19 (2 μM), an inhibitor of NAADP receptor activation. GDNF was used as positive control at a concentration of 20 ng/mL. Results are expressed in % of neurons in the undeprived condition. $ P<0.05 vs untreated cultures; # P<0.05 vs cultures treated with GDNF.

FIG. 3 is a bargraph showing the neuroprotective effect of NAD⁺ (1) and several anti-CD38 antibodies (HB7 (2), AT1 (3), clone 90 (4), AT13/5 (5) or OKT-10 (6)) against MPP⁺-induced DA cell death. DA (TH+) cell survival in midbrain cultures treated with MPP⁺ (3 μM) between 5 and 7 DIV exposed or not to NAD⁺ (3 mM) or several anti-CD38 antibodies (clone 90, AT1, AT13/5, OKT-10 or HB-7; 1 μg/mL) in the presence or not of Ned-19 (2 μM), an inhibitor of NAADP receptor activation. Results are expressed in % of corresponding control cultures. $ P<0.05 vs control treatment; # P<0.05 vs MPP⁺ treatment.

FIG. 4 is a bargraph showing the effect of NAD⁺ (1) and several anti-CD38 antibodies (HB7 (2), AT1 (3), clone 90 (4), AT13/5 (5) or OKT-10 (6)) on microglial cells following MPP⁺ (3 μM) treatment. Increase in the number of microglial cells in midbrain cultures treated with MPP⁺ (3 μM) between 5 and 7 DIV exposed or not to NAD⁺ (3 mM) or several anti-CD38 antibodies (clone 90, AT1, AT13/5, OKT-10 or HB-7; 1 μg/mL) in the presence or not of Ned-19 (2 μM), an inhibitor of NAADP receptor activation. Results are expressed in % of corresponding control cultures. $ P<0.05 vs control treatment; # P<0.05 vs MPP⁺ treatment.

FIG. 5 is a bargraph showing the neuroprotective effect of NAD+(1) and several anti-CD38 antibodies (HB7 (2), AT1 (3), clone 90 (4), AT13/5 (5) or OKT-10 (6)) against excitotoxicity triggered by a prolonged treatment of cortical cultures with glutamate (75 μM). Neuronal cell survival in cortical cultures treated with glutamate (75 μM) between 12 and 14 DIV and exposed to NAD⁺ (3 mM) or several anti-CD38 antibodies (clone 90, AT1, AT13/5, OKT-10 or HB-7; 1 μg/mL) in the presence or not of Ned-19 (2 μM), an inhibitor of NAADP receptor activation. Results are expressed in % of corresponding control cultures. $ P<0.05 vs glutamate treatment; # P<0.05 vs control treatment.

FIG. 6 is a bargraph showing the neuroprotective effect of NAD⁺ (1) and several anti-CD38 antibodies (HB7 (2), AT1 (3), clone 90 (4), AT13/5 (5) or OKT-10 (6)) against oxidative stress triggered by a treatment of cortical cultures with H₂O₂ (75 μM). Neuronal cell survival in cortical cultures treated with H₂O₂ (75 μM) between 12 and 14 DIV and exposed to NAD⁺ (3 mM) or several anti-CD38 antibodies (clone 90, AT1, AT13/5, OKT-10 or HB-7; 1 μg/mL) in the presence or not of Ned-19 (2 μM), an inhibitor of NAADP receptor activation. Results are expressed in % of corresponding control cultures. $ P<0.05 vs H₂O₂ treatment; # P<0.05 vs control treatment.

FIG. 7 is a bargraph showing the effect of HB7 antibody on cytosolic calcium levels in the presence or absence of Ned-19 (2 μM). Increase in cytosolic calcium levels in cortical cultures between 7 and 10 DIV exposed or not to HB-7 (1 μg/mL) in the presence or not of Ned-19 (2 μM), an inhibitor of NAADP receptor activation. Results are expressed in % of corresponding control cultures. $ P<0.05 vs control treatment.

FIG. 8 is a bargraph showing the neuroprotective effect of ara-2′-F-NAD⁺ (ARA-F-NAD, 200 μM) against MPP⁺-induced DA cell death. DA (TH⁺) cell survival in midbrain cultures treated with MPP⁺ (3 μM) between 5 and 7 DIV exposed or not to ARA-F-NAD in the presence or not of Ned-19 (2 μM), an inhibitor of NAADP receptor activation. Results are expressed in % of corresponding control cultures. $ P<0.05 vs control treatment; # P<0.05 vs MPP⁺ treatment.

FIG. 9 is a bargraph showing the neuroprotective effect of anti-CD38 clone HB7 antibody (1), NAD⁺ (2) and cADPR (3) against MPP⁺-induced DA cell death. DA (TH⁺) cell survival in midbrain cultures treated with MPP+(3 μM) between 5 and 7 DIV exposed or not to anti-CD38 clone HB7 antibody (1 μg/mL), NAD⁺ (3 mM) or cADPR (200 μM) in the presence or not of Ned-19 (2 μM), an inhibitor of NAADP receptor activation, the inhibitors of lysosomal maturation and of Ca²⁺-dependent lysosomal exocytosis vacuolin-1 (VAC, 10 μM) and endosidin2 (ENDO, 40 μM), the sirtuin-1 inhibitor Ex-527 (EX527, 100 μM), or the ryanodine receptor antagonist dantrolene (DANT, 30 μM). Results are expressed in % of corresponding control cultures. $ P<0.05 vs reference treatment (HB7, NAD⁺ or cADPR) alone.

FIG. 10 is a cartoon depicting the neuroprotective mechanism of action of anti-CD38 clone HB7 antibody (1), NAD⁺ (2) and cADPR (3). ER: endoplasmic reticulum. RyR: ryanodine receptor.

FIG. 11 is a bargraph showing the effect of anti-CD38 clone HB7 antibody (1) and NAD⁺ (2) on cytosolic calcium levels in the presence or absence of the endocytosis inhibitor jasplakinolide or the lysosomal acidification inhibitor bafilomycinA1. Increase in cytosolic calcium levels in cortical cultures between 7 and 10 DIV exposed or not to anti-CD38 clone HB-7 antibody (1 μg/mL) or NAD⁺ (3 mM) in the presence or not of jasplakinolide (JAS, 10 μM), an inhibitor of endocytosis, or bafilomycinA1 (BAF, 50 nM), an inhibitor of lysosomal acidification. Results are expressed in % of corresponding control cultures. $ P<0.05 vs reference treatment (HB7 or NAD⁺) alone.

FIG. 12 is a bargraph showing the neuroprotective effect of anti-CD38 clone HB7 antibody (1) and NAD⁺ (2) against MPP⁺-induced DA cell death. DA (TH⁺) cell survival in midbrain cultures treated with MPP⁺ (3 μM) between 5 and 7 DIV exposed or not to anti-CD38 clone HB7 antibody (1 μg/mL) or NAD⁺ (3 mM) in the presence or not of jasplakinolide (JAS, 10 μM), an inhibitor of endocytosis. Results are expressed in % of corresponding control cultures. $ P<0.05 vs reference treatment (HB7 or NAD⁺) alone.

FIG. 13 is a bargraph showing the effect of acute (30 minutes) treatment with insulin, anti-CD38 clone HB7 antibody and NAD+ on glucose uptake in cortical neurons. Increase in glucose uptake in cortical cultures at 7 DIV exposed or not to insulin (100 nM), anti-CD38 clone HB7 antibody (1 μg/mL) or NAD⁺ (3 mM). Results are expressed in % of corresponding control cultures. $ P<0.05 vs control treatment.

FIG. 14 is a bargraph showing the effect of three different concentrations of anti-CD38 HB7 antibody (0.1, 0.4 and 1 mg/kg) intracerebrally injected in the in vivo unilateral 6-OHDA mouse model on the number of DA (TH⁺) neurons in the substantia nigra pars compacta (1), on striatal dopamine levels (2), on apomorphine-induced contralateral rotations (3) and on plasma interleukin-10 (IL-10) levels (4). Results are expressed in % of non-lesioned side (1, 2), number of contralateral rotations (3) or plasma IL-10 levels expressed in pg/mL (4). Control group: n=3; 6-OHDA group: n=7; 6-OHDA+HB7 0.1 mg/kg icy (intracerebroventricular injection) group: n=9; 6-OHDA+HB7 0.4 mg/kg icy group: n=14; 6-OHDA+HB7 1 mg/kg icy group: n=9. $ P<0.05 vs control (non-injected) group; # P<0.05 vs 6-OHDA group.

FIG. 15 is a bargraph showing the neuroprotective effect of anti-CD38 HB7 antibody injected either intracerebrally (1 mg/kg icy, concomitantly with 6-OHDA) or intravenously (4 mg/kg iv, injected one day before stereotaxic injection of 6-OHDA) in the in vivo unilateral 6-OHDA mouse model on the number of DA (TH⁺) neurons in the substantia nigra pars compacta. Results are expressed in % of non-lesioned side. PBS group: n=9; 6-OHDA group: n=10; 6-OHDA+HB7 1 mg/kg icy group: n=7; 6-OHDA+HB7 4 mg/kg iv group: n=9. $ P<0.05 vs control (PBS-injected) group; # P<0.05 vs 6-OHDA group.

FIG. 16 is a bargraph showing the effect of anti-CD38 HB7 antibody (1 mg/kg) injected intracerebrally either concomitantly with 6-OHDA (D0), one day (D1) or two days after 6-OHDA lesion in the in vivo unilateral 6-OHDA mouse model on the number of DA (TH⁺) neurons in the substantia nigra pars compacta. Results are expressed in % of non-lesioned side. PBS group: n=14; 6-OHDA group: n=16; 6-OHDA D0+HB7 1 mg/kg icy D0 group: n=18; 6-OHDA D0+HB7 1 mg/kg icy D1 group: n=6; 6-OHDA D0+HB7 1 mg/kg icy D2 group: n=18. $ P<0.05 vs control (PBS-injected) group; # P<0.05 vs 6-OHDA group.

FIG. 17 is a bargraph showing the effect of anti-CD38 HB7 antibody or anti-CD38 OKT10 antibody injected intracerebrally (1 mg/kg icy, concomitantly with 6-OHDA) in the in vivo unilateral 6-OHDA mouse model on the number of DA (TH+) neurons in the substantia nigra pars compacta. Results are expressed in % of non-lesioned side. PBS group: n=9; 6-OHDA group: n=10; 6-OHDA+HB7 1 mg/kg icy group: n=10; 6-OHDA+OKT10 1 mg/kg group: n=10. $ P<0.05 vs control (PBS-injected) group. # P<0.05 vs 6-OHDA group.

FIG. 18 is a picture (1), a bargraph (2) and a point plot (3) showing the effect of anti-CD38 HB7 antibody injected either intracerebrally (1 mg/kg icy, injected one day before the beginning of CBE treatment) or intravenously (4 mg/kg iv, injected one day before the beginning of CBE treatment) on the number (1, 2) and the area (1, 3) occupied by striatal microglial cells in the in vivo CBE mouse model. Results are expressed in % of sham (saline-injected) group. Sham group: n=8; CBE group: n=9; CBE+HB7 1 mg/kg icy group: n=8; CBE+HB7 4 mg/kg iv group: n=7. $ P<0.05 vs sham (saline-injected) group; # P<0.05 vs CBE group.

FIG. 19 is a picture (1) and a point plot (2) showing the effect of anti-CD38 HB7 antibody injected either intracerebrally (1 mg/kg icy, injected one day before the beginning of CBE treatment) or intravenously (4 mg/kg iv, injected one day before the beginning of CBE treatment) on the area occupied by striatal reactive astrocytes in the in vivo CBE mouse model. Results are expressed in % of sham (saline-injected) group. Sham group: n=8; CBE group: n=9; CBE+HB7 1 mg/kg icy group: n=8; CBE+HB7 4 mg/kg iv group: n=7. $ P<0.05 vs sham (saline-injected) group; # P<0.05 vs CBE group.

FIG. 20 is a bargraph showing the effect of anti-CD38 HB7 antibody injected intravenously (15 mg/kg iv, two days before MPTP injections) in the in vivo MPTP mouse model on the number of DA (TH⁺) neurons in the substantia nigra pars compacta. Results are expressed in % of control (PBS-injected) mice. PBS group: n=5; MPTP group: n=6; MPTP+HB7 15 mg/kg iv group: n=8. P<0.05 vs control (PBS-injected) group. # P<0.05 vs MPTP group.

### EXAMPLES

The present invention is further described in the following examples, but the technical scope of the present invention is not limited to these examples.

TABLE 1
summarizes information, in particular, the epitope binding sequence within CD38 (of amino acid sequence SEQ ID NO: 1),
of the antibodies used in the examples.

| | | | | | Effect on | | |
|---|---|---|---|---|---|---|---|
| CD38 | | | | | CD38 NADase | Effect on | |
| antibody | Immunogen | Commercial | Epitope sequence | | (glycohydrolase) | CD38 cyclase | |
| Name | species | reference | binding (*) | Reference | activity | activity | Reference |
| Clone 90 | Mouse | BioLegend | unknown | | None on cell | Inhibit | Hara-Yokoyama et al. |
| | | Ref.: 102702 | | | surface CD38 | | (2008. Int Immunophamacol. |
| | | | | | | | 8(1):59-70) |
| AT1 | Human | Santa Cruz | 5^{th} C-terminal | Ferrero et al. | unknown | unknown | |
| | | Biotechnology | disulfide loop | (2004. BMC | | | |
| | | Ref.: sc-7325 | involving cysteines | Immunol. 5:21) | | | |
| | | | 254 and 275 and | | | | |
| | | | cysteines 254 and 275 | | | | |
| AT13/5 | Human | Santa Cruz | 273-300 | Ellis et al. (1995. J | unknown | none | Ellis et al. (1995. J |
| | | Biotechnology | | Immunol. | | | Immunol. 155(2):925-37); |
| | | Ref.: sc-59028 | | 155(2):925-37) | | | Deckert et al. |
| | | | | | | | (2014. Clin Cancer |
| | | | | | | | Res. 20(17):4574-83) |
| HB7 | Human | BioLegend | 220-285 and | Hoshino et al. | none | none | Deckert et al. (2014. |
| | | Ref.: 356602 | 5^{th} C-terminal | (1997. J Immunol. | | | Clin Cancer Res. |
| | | | disulfide loop | 158(2):741-7); | | | 20(17):4574-83); |
| | | | involving cysteines | Zhao et al. (2011. | | | Hoshino et al. (1997. J |
| | | | 254 and 275 and | J Biol Chem. | | | Immunol. 158(2):741-7) |
| | | | cysteines 254 and 275 | 286(25):22170-7) | | | |
| OKT10 | Human | Anticorps en | 285-300 and | Ferrero et al. | unknown | none | Deckert (Clinic Cancer |
| | | ligne | 6^{th} C-terminal | (2004. BMC | | | Research, 2014, |
| | | Ref.: | disulfide loop | Immunol. 5:21) | | | 20(17):4574-83 |
| | | ABIN2704258 | involving cysteines | | | | |
| | | | 287 and 296 | | | | |

(*) The numbering of amino acids of human CD38 consisting of said “epitope sequence binding” corresponds to the numbering of amino acids of the human CD38 sequence set forth in SEQ ID NO: 1 and referenced by the NP_001766 NCBI accession number.

### Example 1: In Vitro Neuroprotection of Midbrain Dopaminergic Neurons by Anti-CD38 Antibodies

Prevention of Spontaneous and Progressive Dopaminergic (DA) Cell Death in Midbrain Cultures

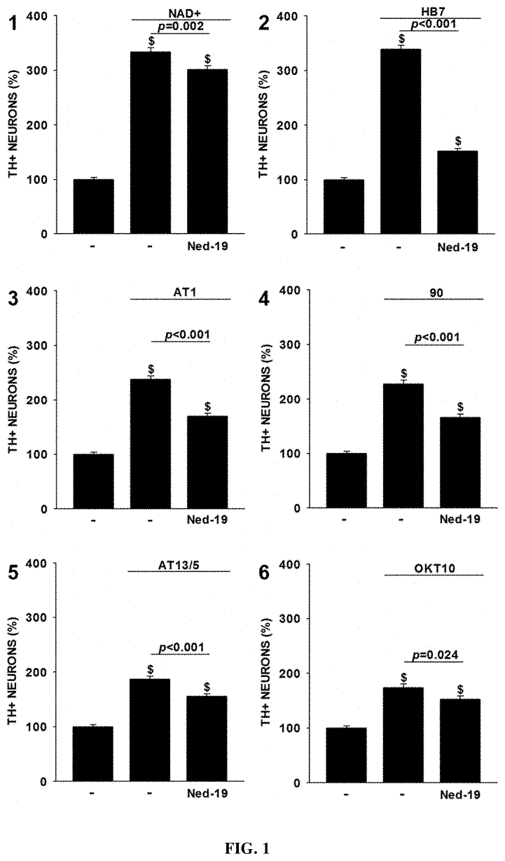

We report here that NAD⁺ (3 mM) and several anti-CD38 antibodies (clone 90, AT1, AT13/5, OKT-10 or HB-7; 1 μg/mL) increased the number of midbrain DA (TH⁺) neurons in culture conditions where these neurons die spontaneously, selectively and progressively as they mature (FIG. 1). Noteworthy, the neuroprotective effect of anti-CD38 antibodies (clone 90, AT1, AT13/5, OKT-10 or HB-7), and slightly but significantly that of NAD⁺, was antagonized in the presence of Ned-19 (2 μM), an inhibitor of NAADP receptor activation.

Protection of DA Neurons that Degenerate after GDNF Deprivation

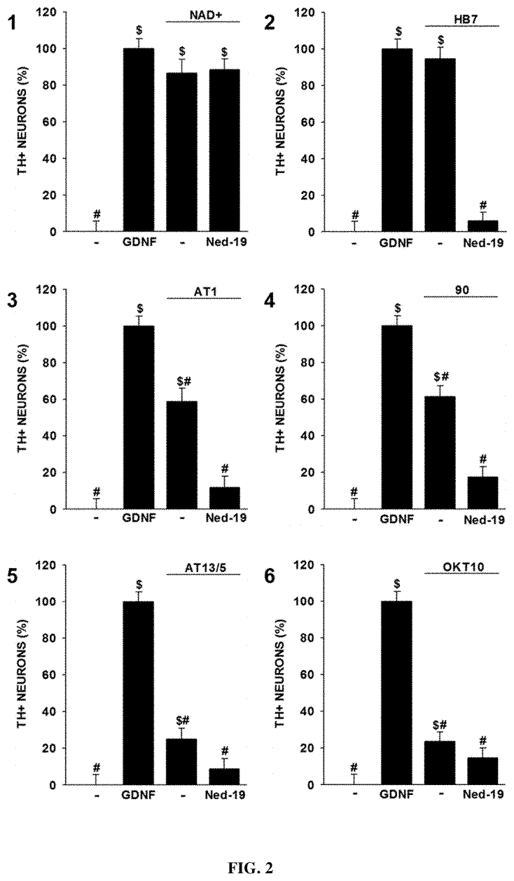

To determine whether the protective effect of NAD⁺ (3 mM) and several anti-CD38 antibodies (clone 90, AT1, AT13/5, OKT-10 or HB-7; 1 μg/mL) remained observable in more mature DA neurons (FIG. 2), we used midbrain cultures in which the spontaneous death process was postponed for 10 days by chronic application of GDNF (20 ng/mL). Ablation of GDNF from these cultures at 11 days in vitro (DIV) led to a massive and selective loss of TH⁺ neurons within the next 5 days in agreement with previous data (Guerreiro et al., 2008. Mol Pharmacol. 74(4):980-9). Interestingly, a large population of these neurons were saved from death by NAD⁺ (3 mM) or anti-CD38 antibodies (clone 90, AT1, AT13/5, or HB-7; 1 μg/mL) but not by clone OKT-10 anti-CD38 antibody (1 μg/mL). The neuroprotective effect of anti-CD38 antibodies clone 90, AT1, AT13/5, or HB-7 but not that of NAD⁺ was abolished in the presence of Ned-19 (2 μM), an inhibitor of NAADP receptor activation.

Protection Against the Mitochondrial Neurotoxin MPP⁺

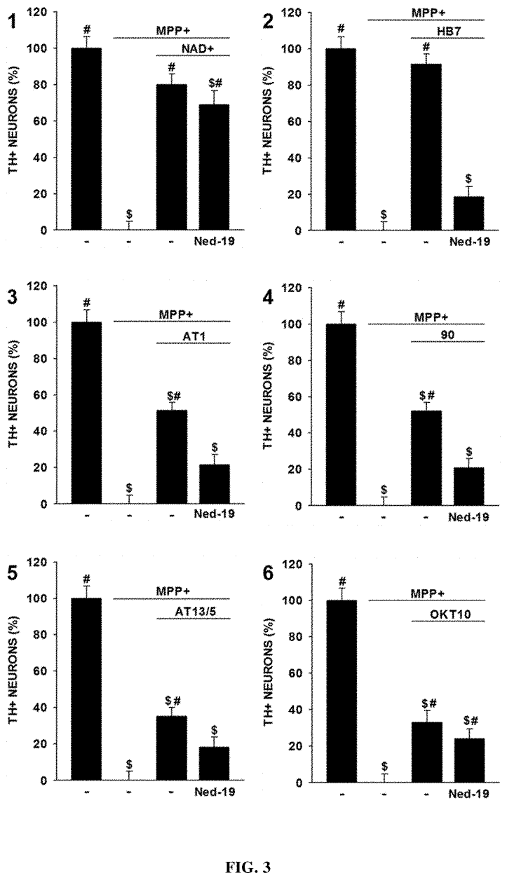

To determine whether NAD⁺ (3 mM) and several anti-CD38 antibodies (clone 90, AT1, AT13/5, OKT-10 or HB-7; 1 μg/mL) remained protective in a situation in which DA cell death was caused by mitochondrial poisoning with MPP+, the active metabolite of the DA neurotoxin MPTP was tested. For this purpose, spontaneously occurring DA cell death was prevented by a treatment combining depolarizing concentrations of K⁺ (30 mM) and MK801 (5 μM), a glutamate receptor antagonist used to prevent unwanted excitotoxic insult. The cultures were then exposed to 3 μM MPP⁺ between 5 and 7 DIV to achieve a loss of approximately 50% of DA neurons. When the cultures were exposed to NAD⁺ (3 mM) or anti-CD38 antibodies (clone 90, AT1, AT13/5, OKT-10 or HB-7; 1 μg/mL) during the intoxication period, MPP⁺-induced DA cell loss was either partially or almost totally prevented by these compounds (FIG. 3). The neuroprotective effect of anti-CD38 antibodies clone 90, AT1, AT13/5, or HB-7 but not that of NAD⁺ or clone OKT-10 anti-CD38 antibody (1 μg/mL) was abolished in the presence of Ned-19 (2 μM), an inhibitor of NAADP receptor activation.

Also note that these effects are true, direct neuroprotective effects and are not due to a possible interference with CD38 localized on immune cells (as illustrated in Hara-Yokoyama et al., 2008. Int Immunopharmacol. 8(1):59-70) since there are no peripheral immune cells in this in vitro cell culture setting.

### Example 2: In Vitro Anti-Inflammatory Effect of Anti-CD38 Antibodies

Limitation of the Number of Microglial Cells

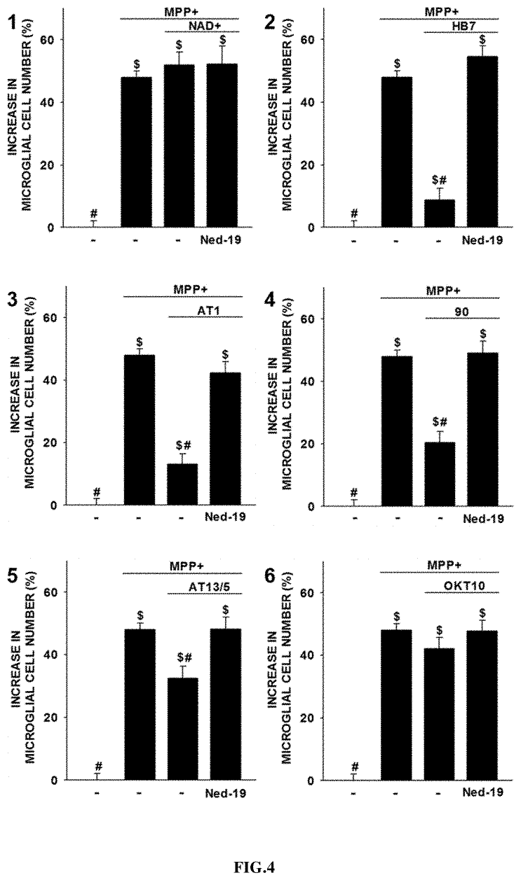

Neuro-inflammation has been repeatedly implicated in neurodegeneration and microglial cells, the resident innate immune cells in the brain, may play a key role in this process. In vitro, MPP⁺ was shown to induce an increase in microglial cells number (Henze et al., 2005. J Neurochem. 95(4):1069-77). We tested whether NAD⁺ (3 mM) and several anti-CD38 antibodies (clone 90, AT1, AT13/5, OKT-10 or HB-7; 1 μg/mL) were able to reduce the increase in the number of microglial cells observed following MPP⁺ application in vitro. We observed that anti-CD38 antibodies (clone 90, AT1, AT13/5 or HB-7; 1 μg/mL) but not NAD⁺ (3 mM) or clone OKT-10 anti-CD38 antibody (1 μg/mL) reduced the number of microglial cells in 7 DIV midbrain cultures (FIG. 4) and that this effect was suppressed when Ned-19 (2 μM) was concomitantly added to the cultures.

### Example 3: In Vitro Neuroprotection of Cortical Neurons by Anti-CD38 Antibodies

Protection from Excitotoxicity

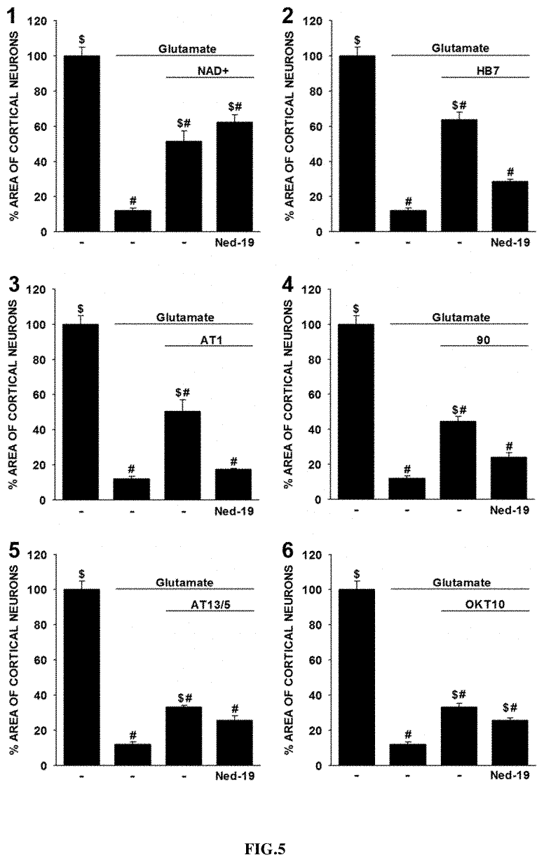

Excitotoxicity occurring through an over-activation of glutamate receptors is thought to play a key role in neurodegeneration (Lewerenz and Maher, Front Neurosci, 2015, 9:469). To determine whether NAD⁺ (3 mM) and several anti-CD38 antibodies (clone 90, AT1, AT13/5, OKT-10 or HB-7; 1 μg/mL) protected neurons from excitotoxic insults, we tested these compounds in a model in which neurodegeneration is induced in cortical cultures following glutamate (75 μM) exposure. We observed that cultures exposed to NAD⁺ (3 mM) or anti-CD38 antibodies (clone 90, AT1, AT13/5, OKT-10 or HB-7; 1 μg/mL) were protected from excitotoxicity (FIG. 5). The neuroprotective effect of anti-CD38 antibodies clone 90, AT1, and HB7 but not that of NAD⁺ or anti-CD38 antibodies clone AT13/5 or OKT-10 (1 μg/mL) was abolished in the presence of Ned-19 (2 μM), an inhibitor of NAADP receptor activation.

Protection from Oxidative Stress

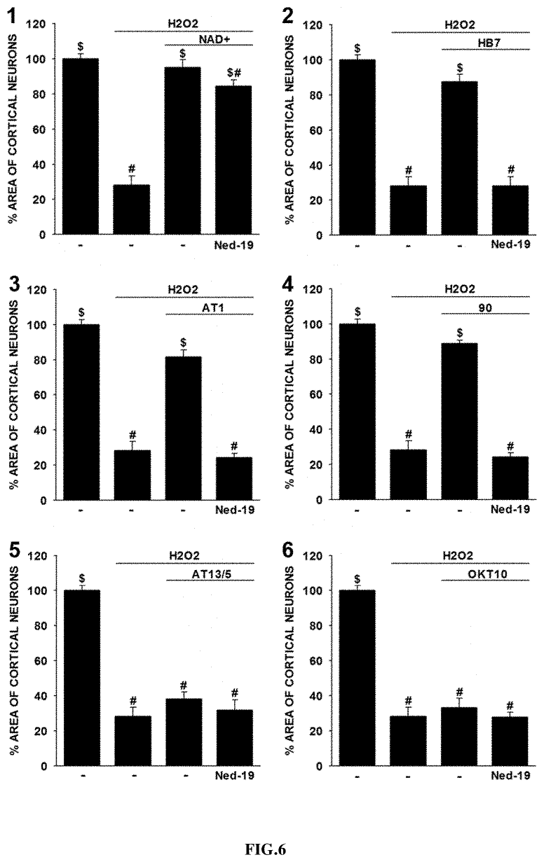

To determine whether NAD⁺ (3 mM) and several anti-CD38 antibodies (clone 90, AT1, AT13/5, OKT-10 or HB-7; 1 μg/mL) protected neurons from oxidative stress insults, we tested these compounds in a model in which neurodegeneration is induced in cortical cultures following H₂O₂ (75 μM) exposure. We observed that cultures exposed to NAD⁺ (3 mM) or anti-CD38 antibodies (clone 90, AT1 or HB-7; 1 μg/mL) but not those exposed to anti-CD38 antibodies clone AT13/5 or OKT-10 (1 μg/mL) were protected from oxidative stress (FIG. 6). The neuroprotective effect of anti-CD38 antibodies clone 90, AT1, and HB7 but not that of NAD⁺ was abolished in the presence of Ned-19 (2 μM), an inhibitor of NAADP receptor activation.

Neuroprotection is Associated with an Increase in Intracellular Free Calcium Levels

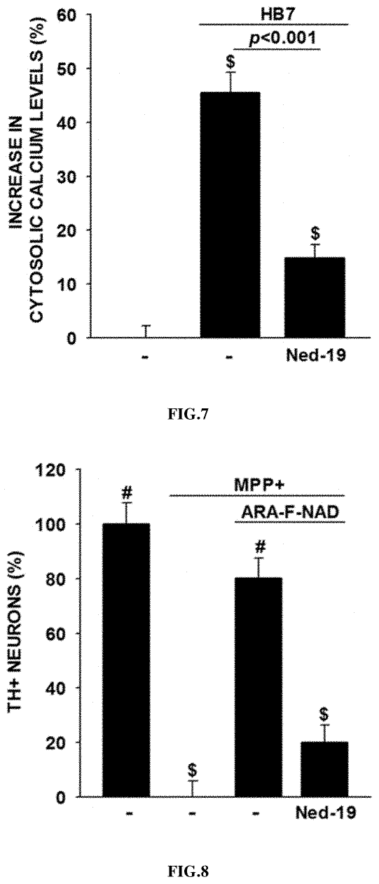

The NAADP receptors TPC1 and TPC2 are known to release calcium from lysosomal compartment, leading to an increase in cytoplasmic calcium (Pitt et al., 2016. J Physiol. 594(15):4171-9). To determine whether cytosolic calcium levels are increased following anti-CD38 clone HB7 antibody (1 μg/mL) treatment through the recruitment of NAADP receptors, we measured cytosolic calcium levels of cortical neurons in the presence or not of HB7 and/or the NAADP receptor antagonist Ned-19 (2 μM) (FIG. 7). We observed that anti-CD38 clone HB7 antibody increased cytosolic calcium levels, and that this effect is abrogated in the presence of the NAADP receptor antagonist Ned-19 (2 μM).

### Example 4: In Vitro Neuroprotection of Midbrain Dopaminergic Neurons by Small Organic Molecule CD38 Inhibitor

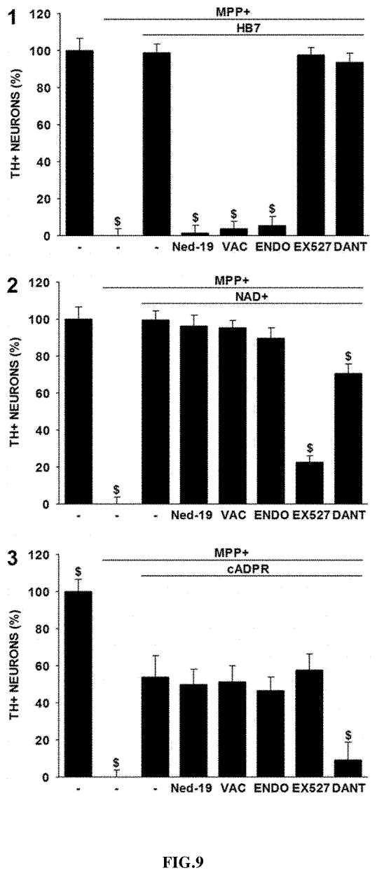

To determine whether small organic molecule inhibitor of CD38 enzymatic activity were neuroprotective, we tested the effect of ara-2′-F-NAD+ (ARA-F-NAD), a slow-binding and selective inhibitor of CD38 (Muller-Steffner et al., 1992. J Biol Chem. 267(14):9606-11; Berthelier et al., 1998. Biochem J. 330(Pt 3):1383-90), in the MPP⁺ in vitro model. When the cultures were exposed to ARA-F-NAD (200 μM) during the intoxication period, MPPtinduced DA cell loss was almost totally prevented by this CD38 inhibitor (FIG. 8). The neuroprotective effect induced by ARA-F-NAD was abolished in the presence of Ned-19 (2 μM), an inhibitor of TPC 1 and/or 2 activation. This experiment demonstrates that this small organic molecule CD38 inhibitor is able to activate the opening of NAADP receptors TPC1 and/or TPC2, leading to protection of neurons.

### Example 5: Comparative Analysis of the Neuroprotective Effect of Anti-CD38 Clone HB7, NAD⁺ and cADPR

CD38 enzymatic activity is very complex and can lead to the production or the degradation of NAADP, NAD⁺ and cyclic Adenosine DiPhosphate Ribose (cADPR) (Malavasi et al., 2008. Physiol Rev. 88(3):841-86). We previously observed that the effects of anti-CD38 clone HB7 were antagonized in the presence of an inhibitor of TPC 1 and/or 2 activation, Ned-19 (2 μM), while the effects of NAD⁺ were not suggesting that the neuroprotective effect of anti-CD38 clone HB7 did not involve increases in NAD⁺ levels. To deeper investigate whether the neuroprotective effect of anti-CD38 clone HB7 indeed involved increased levels of NAD⁺ or cADPR or not, we studied the mechanism of action of neuroprotection of each of these compounds.

Neuroprotection by Anti-CD38 Clone HB7 does not Involve NAD⁺ or cADPR

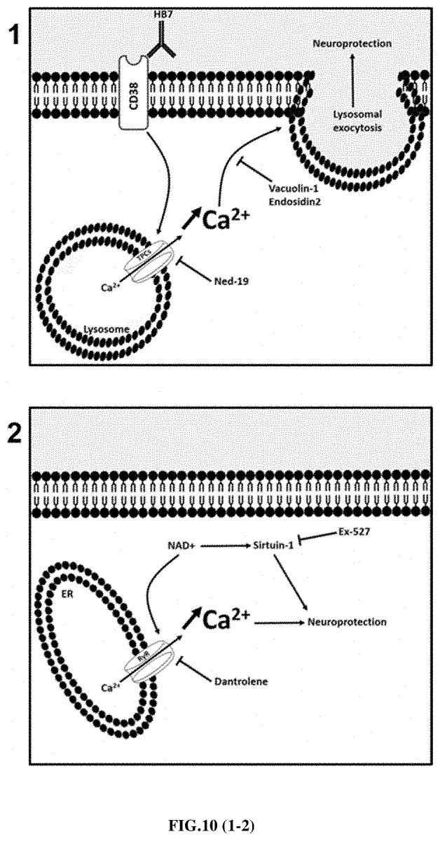

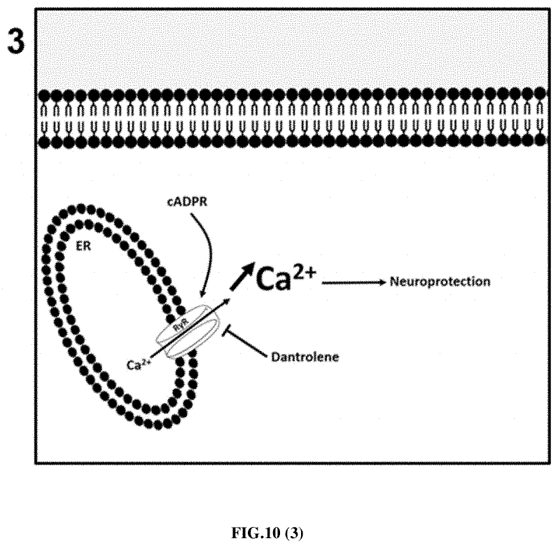

We first studied the intracellular mechanism of action of neuroprotection of anti-CD38 clone HB7 (1 μg/mL), NAD⁺ (3 mM) or cADPR (200 μM) in the MPP⁺ in vitro model in the presence or the absence of the inhibitor of TPC 1 and/or 2 activation Ned-19, the inhibitors of lysosomal maturation and of Ca²⁺-dependent lysosomal exocytosis vacuolin-1 and endosidin2, the sirtuin-1 inhibitor Ex-527, or the ryanodine receptor antagonist dantrolene (FIG. 9). We observed that the neuroprotective effect of anti-CD38 clone HB7 antibody (1 μg/mL) was fully antagonized in the presence of Ned-19 (2 μM), vacuolin-1 (10 μM) or endosidin2 (40 μM) while the neuroprotective effect of NAD⁺ (3 mM) was only antagonized in the presence of Ex-527 (100 μM) or dantrolene. The neuroprotective effect of cADPR (200 μM) was only antagonized in the presence of dantrolene. These results suggest that the neuroprotective effect of cADPR depends on the opening of Ryanodine receptors as previously described (Ogunbayo et al., 2011. J Biol Chem. 286(11):9136-40), while the neuroprotective effect of NAD involves the recruitment of Sirtuin-1 and Ryanodine receptors as previously described (Ng et al., 2015. Front Neurosci. 9: 64). Contrarily, we found that the neuroprotective effect of anti-CD38 clone HB7 antibody triggered a complex cascade of events involving TPCs opening and subsequent calcium efflux from the lysosome to the cytosol that will lead to lysosomal exocytosis (FIG. 10). Altogether, these results indicated that neuroprotection by anti-CD38 clone HB7 antibody did not involve NAD⁺ or cADPR since it is not antagonized in the presence of Ex-527 or dantrolene.

Neuroprotection and Increase in Cytosolic Ca²⁺ Following Anti-CD38 Clone HB7 Requires CD38 Internalization and Lysosomal Acidic Environment

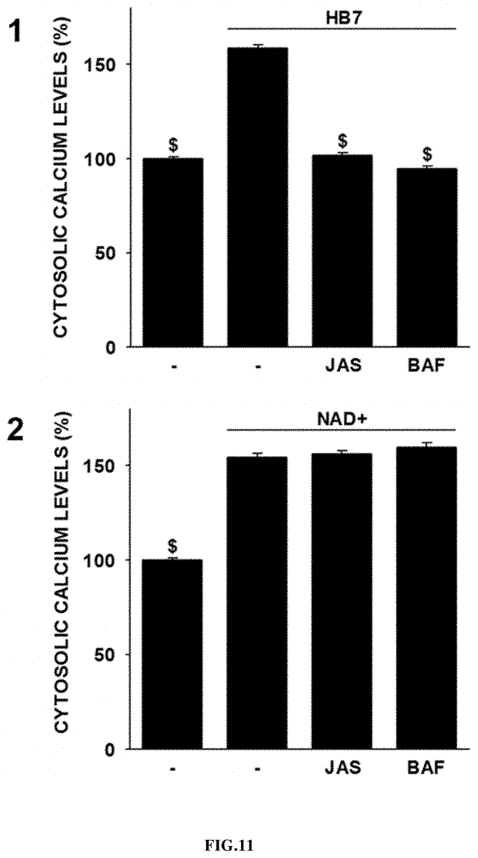

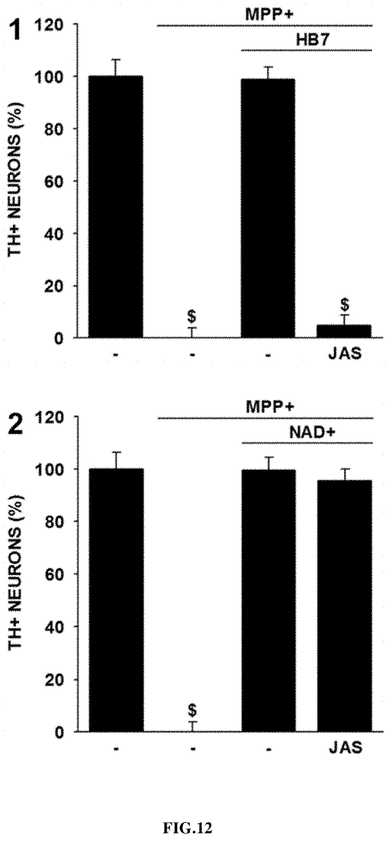

Several articles demonstrated that CD38 binding with certain antibodies triggered CD38 internalization and relocalization to the lysosome (Funaro et al., 1998. J Immunol. 160(5):2238-47). Moreover, NAADP synthesis by CD38 only occurs at acidic pH (Aarhus et al., 1995. J Biol Chem. 270(51):30327-33). Such pH conditions can only be found intracellularly in the lysosomal compartment. To determine whether the increase in cytosolic Ca²⁺ levels observed following anti-CD38 clone HB7 or NAD⁺ required CD38 internalization and/or lysosomal acidic environment, we monitored cytosolic Ca²⁺ levels in the presence of the inhibitor of endocytosis jasplakinolide (10 μM) or the inhibitor of lysosomal acidification bafilomycinA1 (50 nM) (FIG. 11). We observed that the increase in cytosolic Ca²⁺ levels obtained following anti-CD38 clone HB7 treatment (1 μg/mL) was fully antagonized in the presence of jasplakinolide or bafilomycinA1, while the effect of NAD⁺ (3 mM) was not. Moreover, the neuroprotective effect of anti-CD38 clone HB7 in the MPP⁺ model was also antagonized in the presence of jasplakinolide, while the neuroprotective effect of NAD⁺ was not (FIG. 12). These results suggest that anti-CD38 clone HB7 neuroprotection and increase in cytosolic Ca²⁺ levels
(i) is different from that of NAD⁺ and
(ii) are mediated by triggering CD38 internalization, redirection to the lysosome, and synthesis of NAADP under acidic pH conditions.

This mechanism of action has been recently described elsewhere (Fang et al., 2018. J Biol Chem. 293(21):8151-8160) but not associated to neuroprotection.

Acute Treatment with Anti-CD38 Clone HB7 Antibody Strongly Increased Glucose Uptake in Cortical Cultures

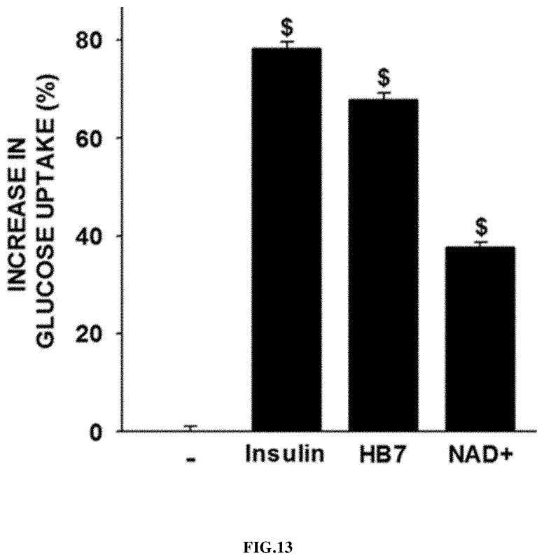

The NAADP-CD38 axis was previously shown to control glucose uptake in adipocytes (Song et al., 2012. Cell Rep. 2(6):1607-19). To demonstrate whether this effect could also be observed in neurons, we evaluated the increase in glucose uptake induced following acute treatment (30 min) with insulin, anti-CD38 clone HB7 antibody or NAD+ using the fluorescent glucose analogue 2-NBDG in cortical neuron cultures. We observed that acute treatment with anti-CD38 clone HB7 antibody (1 μg/mL) increased glucose uptake to the same extent that insulin (100 nM) did (FIG. 13). NAD⁺ (3 mM) treatment only modestly increased glucose uptake in cortical cultures.

Of note, glucose hypometabolism has been shown to be a prominent feature in the brain of subjects affected with neurodegenerative diseases (Li et al., 2012. Biochem Biophys Res Commun. 421(4):727-30; Hassan et al., 2014. CNS Neurol Disord Drug Targets. 13(7):1232-45; Niccoli et al., 2016. Curr Biol. 26(17):2291-300).

The results herein therefore strongly support the effect of anti-CD38 antibodies for the treatment of neurodegenerative diseases, through regulation of the glucose metabolism.

### Example 6: In Vivo Neuroprotection of Midbrain Dopaminergic Neurons in the Unilateral 6-OHDA Mouse Model

Dose Response Study of Anti-CD38 Clone HB7 Treatment in the Unilateral 6-OHDA Mouse Model

To determine whether anti-CD38 clone HB7 antibody was neuroprotective in vivo against neurodegeneration induced by oxidative stress, we intracerebrally injected different concentrations of this antibody (0.1, 0.4 or 1 mg/kg) in a mouse model in which dopaminergic neurons of the right substantia nigra pars compacta degenerate following administration of 6-hydroxydopamine (6-OHDA) in the right striatum. Since 6-OHDA is an analogue of dopamine, it is transported in dopaminergic neurons through the dopamine transporter, resulting in a specific cell death of dopaminergic population. It has been used extensively as a test system for novel symptomatic agents and for assessment of neuroprotective and neurorepair strategies (Galindo et al., 2014. In Kostrzewa (Ed.), Handbook of neurotoxicity (1st Ed., pp. 639-651). New York: Springer-Verlag). Since 6-OHDA is injected unilaterally in the right striatum, each mouse is its own control since dopaminergic neurons of the left substantia nigra are not affected by the toxin.

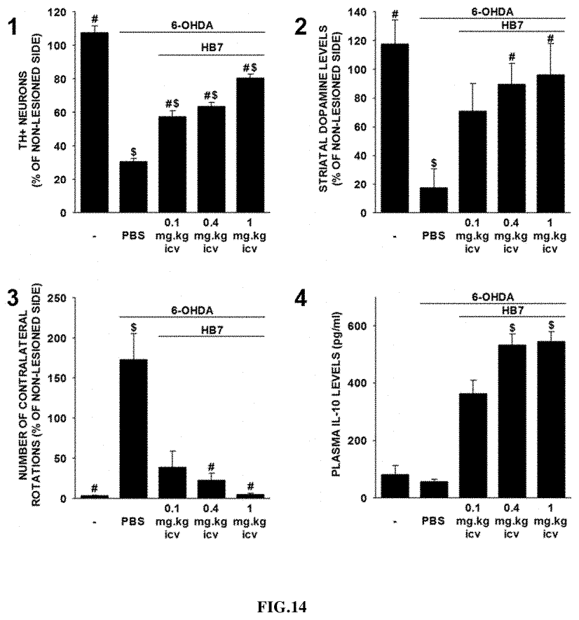

We observed that anti-CD38 clone HB7 antibody dose-dependently protected dopaminergic neurons of the substantia nigra pars compacta (FIG. 14). Moreover, this neuroprotective effect was accompanied by a rescue of dopamine levels in the striatum, thus meaning that anti-CD38 clone HB7 antibody not only protected dopaminergic neurons but also preserved their functionality. The same dose-dependent effect was observed at the behavioral level in the apomorphine-induced contralateral rotation test.

Finally, treatment with anti-CD38 clone HB7 antibody was also found to dose-dependently increase plasmatic levels of interleukin-10 (IL-10), the main anti-inflammatory cytokine, suggesting that this antibody possessed not only strong neuroprotective but also strong anti-inflammatory properties in vivo.

Differential Effect of Intracerebral or Intravenous Injection of Anti-CD38 Clone HB7 in the Unilateral 6-OHDA Mouse Model

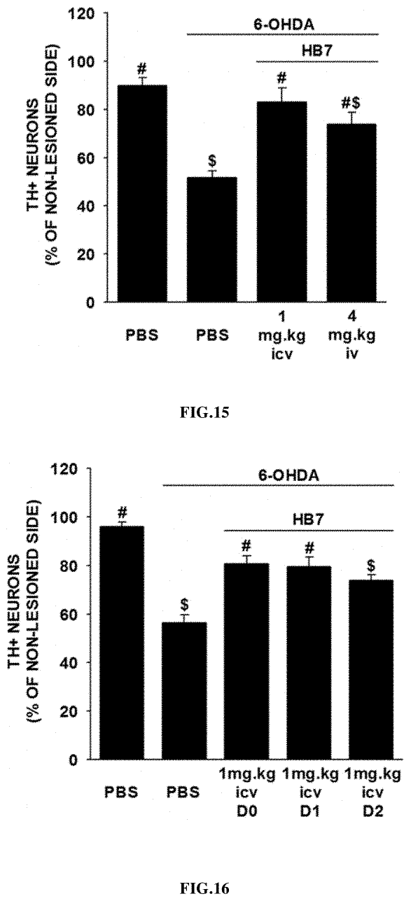

To determine whether anti-CD38 clone HB7 antibody was neuroprotective in vivo following intravenous administration, we compared the neuroprotective effect of anti-CD38 clone HB7 antibody injected either intracerebrally (1 mg/kg) or intravenously (4 mg/kg) in the unilateral 6-OHDA mouse model. We found that anti-CD38 clone HB7 antibody protected dopaminergic neurons of the substantia nigra pars compacta to the same level when injected using either an intravenous or an intracerebral route of administration (FIG. 15).

Effect of Delayed Treatment with Anti-CD38 Clone HB7 in the Unilateral 6-OHDA Mouse Model

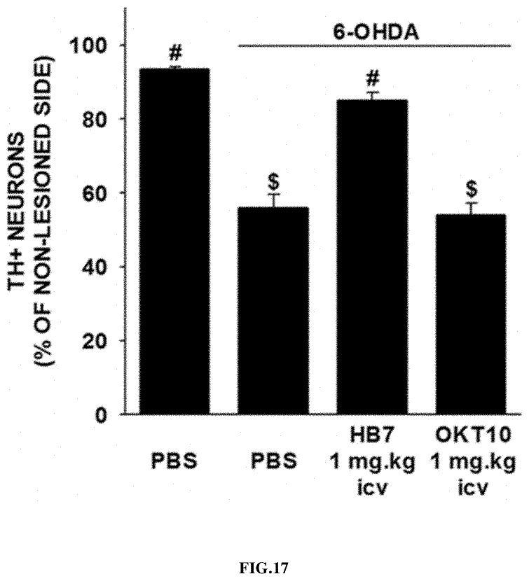

To determine whether anti-CD38 clone HB7 antibody was neuroprotective in vivo even when administered while the neurodegenerative process was ongoing, we compared the neuroprotective effect of anti-CD38 clone HB7 antibody injected intracerebrally (1 mg/kg) either concomitantly to 6-OHDA, one day or two days following 6-OHDA administration in the unilateral 6-OHDA mouse model. We found that anti-CD38 clone HB7 antibody significantly protected dopaminergic neurons of the substantia nigra pars compacta when injected either concomitantly with 6-OHDA or one day following 6-OHDA administration, suggesting that anti-CD38 antibody is still effective in protecting neurons while the neurodegenerative mechanism is already ongoing (FIG. 16).

Of note, when anti-CD38 clone HB7 antibody was injected two days after 6-OHDA injection, the effect was not statistically different from 6-OHDA-treated mice, but a strong trend was observed.

Comparative Effect Between Intracerebral Injection of Anti-CD38 Clone HB7 or Clone OKT10 Antibody in the Unilateral 6-OHDA Mouse Model

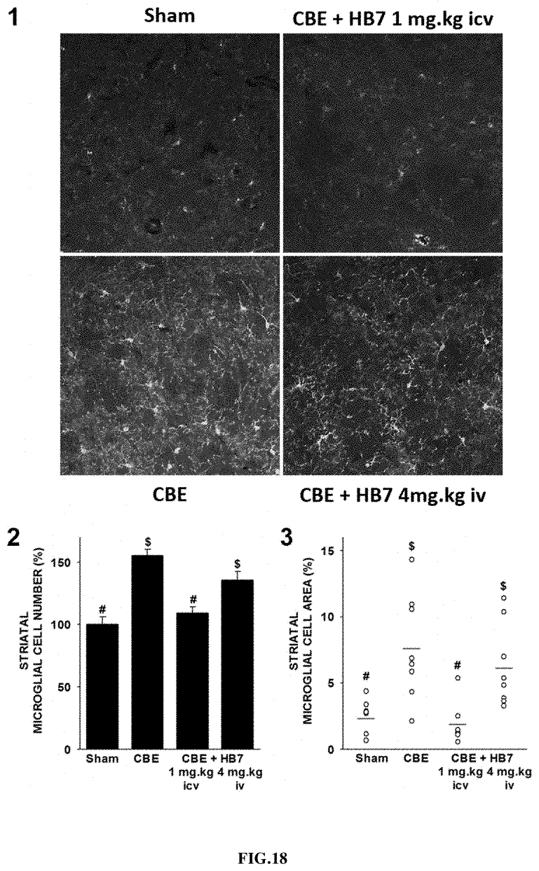

To determine whether anti-CD38 clone OKT10 antibody was neuroprotective in vivo in the unilateral 6-OHDA mouse model, we compared the neuroprotective effect of intracerebral injection (1 mg/kg) of anti-CD38 clone HB7 antibody with that of anti-CD38 clone OKT10 antibody. We observed that anti-CD38 clone OKT10 antibody failed to protect dopaminergic neurons of the substantia nigra pars compacta in the unilateral 6-OHDA mouse model, suggesting that only anti-CD38 antibodies that bind specific epitopes are neuroprotective in vivo (FIG. 17).

Beyond its neuroprotective effect, the dose-dependent increase of the plasmatic level of IL-10, the main anti-inflammatory cytokine, observed upon treatment with the anti-CD38 clone HB7 antibody, indicates its strong anti-inflammatory properties in vivo. This observation suggests a therapeutic interest for anti-CD38 clone HB7 antibodies in the treatment of inflammatory diseases.

### Example 7: In Vivo Anti-Inflammatory Effect in the CBE Mouse Model

Gaucher disease (GD) is an autosomal recessive inborn error of metabolism caused by mutations in the GBA gene coding for the enzyme glucocerebrosidase (GCase). Abnormal GCase function results in the accumulation of glucosylceramide in lysosomes, leading to a variety of systemic manifestations, including organomegaly, anemia, thrombocytopenia, and bone disease. Moreover, different lines of evidence led to the recognition of an unanticipated association between GBA mutations and the development of Parkinson's disease and other Lewy body disorders. Indeed, mutations in the GBA gene constitute numerically the most important predisposing risk factor for developing Parkinson's disease. Both homozygous and heterozygous GBA mutant carrier confer a 20- to 30-fold increased risk for the development of Parkinson's disease, and it is estimated that approximately 5-10% of Parkinson's disease patients have a GBA mutation (Sidransky et al., 2009. N Engl J Med. 361(17):1651-61; Bultron et al., 2010. J Inherit Metab Dis. 33(2):167-73).

An in vivo, chemically-induced mouse model of GD has been developed based on the use of Conduritol β epoxide (CBE), an irreversible inhibitor of GCase that binds its catalytic site (Grabowski et al., 1986. J Biol Chem. 261(18):8263-9), to mimic the loss of mutant GCase enzymatic activity observed in GD patients (Lu et al., 2010. Proc Natl Acad Sci USA. 107(50):21665-70). Of high interest, CBE injected mice displayed strong microglial cells and astrocytes activation (Manning et al., 2009. Neurotoxicology. 30(6):1127-32; Rocha et al., 2015. Antioxid Redox Signal. 23(6):550-642015) due to the lysosomal dysfunctions induced by GCase inhibition.

Effect of Intracerebral or Intravenous Injection of Anti-CD38 Clone HB7 on Striatal Microglial Cells in the CBE Mouse Model.

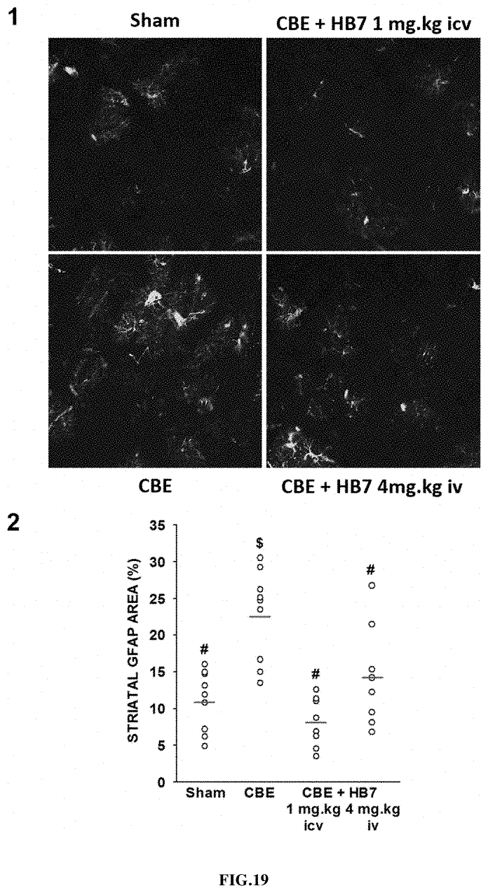

To determine whether anti-CD38 clone HB7 antibody was effective in vivo in counteracting striatal microglial cell activation induced following injection of CBE (100 mg/kg) for 9 consecutive days, animals were injected either intracerebrally (1 mg/kg) or intravenously (4 mg/kg) with anti-CD38 clone HB7 antibody one day prior the first CBE injection. We observed that anti-CD38 clone HB7 antibody intracerebrally injected (1 mg/kg) totally prevented the increase in the number and the area occupied by microglial cells observed following CBE injection alone, while intravenous administration (4 mg/kg) slightly, but not statistically significantly, prevented these effects (FIG. 18).

Effect of Intracerebral or Intravenous Injection of Anti-CD38 Clone HB7 on Striatal Reactive Astrocytes in the CBE Mouse Model.

To determine whether anti-CD38 clone HB7 antibody was effective in vivo in counteracting striatal reactive astrocytes activation induced following injection of CBE (100 mg/kg) for 9 consecutive days, animals were injected either intracerebrally (1 mg/kg) or intravenously (4 mg/kg) with anti-CD38 clone HB7 antibody one day prior the first CBE injection. We observed that anti-CD38 clone HB7 antibody either intracerebrally (1 mg/kg) or intravenously injected totally prevented the increase in the area occupied by reactive astrocytes observed following CBE injection alone (FIG. 19).

### Example 8: In Vivo Neuroprotection of Midbrain Dopaminergic Neurons in the Acute MPTP Mouse Model

To determine whether anti-CD38 clone HB7 antibody was neuroprotective in vivo against neurodegeneration induced by mitochondrial inhibition, we intravenously injected a 15 mg/kg dose of this antibody in a mouse model in which dopaminergic neurons of the substantia nigra pars compacta degenerate following repeated intraperitoneal injection of MPTP. MPTP has the ability to cross the blood brain barrier and is converted into MPP⁺ by the astrocytes. MPP⁺, which is an inhibitor of mitochondrial complex I, is preferentially transported in dopaminergic neurons through the dopamine transporter, resulting in a specific cell death of dopaminergic population. It has been used extensively as a test system for assessment of neuroprotective and neurorepair strategies (Dauer & Przedborski, 2003. Neuron. 39(6): 889-909).

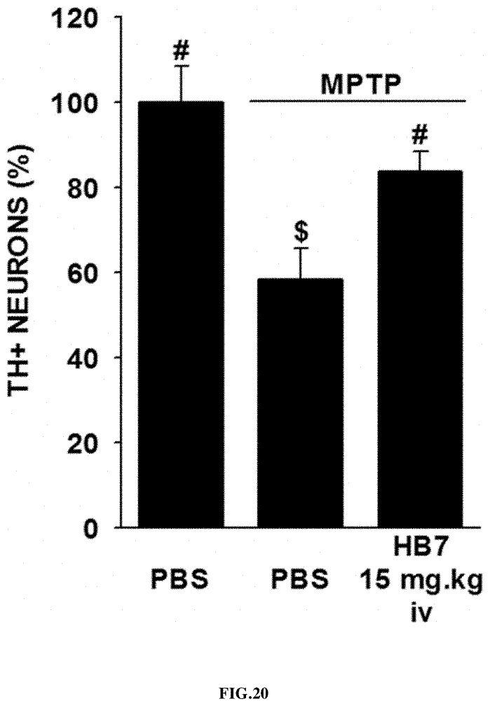

We observed that intravenous injection of anti-CD38 clone HB7 antibody at a 15 mg/kg dose strongly protected dopaminergic neurons of the substantia nigra pars compacta from MPTP-induced neurodegeneration (FIG. 20).

Materials and Methods

In Vitro Experiments

Midbrain Cell Cultures

Animals were treated in accordance with the Guide for the Care and Use of Laboratory Animals (National Research Council, 1996), European Directive 86/609, and the guidelines of the local institutional animal care and use committee. Cultures were prepared from the ventral mesencephalon of gestational age 15.5 Wistar rat embryos (Janvier Breeding Center, Le Genest St Isle, France). Dissociated cells in suspension obtained by mechanical trituration of midbrain tissue pieces were seeded at a density of 1.2-1.5 10⁵ cells/cm² onto tissue culture supports precoated with 1 mg/mL polyethylenimine diluted in borate buffer pH 8.3 as described (Michel et al., 1997. J Neurochem. 69(4):1499-507). The cultures were then maintained in N5 medium supplemented with 5 mM glucose, 5% horse serum, and 0.5% fetal calf serum, except for the first 3 DIV, when the concentration of fetal calf serum was 2.5% to favor initial maturation of the cultures (Guerreiro et al., 2008. Mol Pharmacol. 74(4):980-9). They were fed daily by replacing 70% of the medium. Routinely, mesencephalic cultures were established on Nunc 24-well culture plates (Thermofischer Scientific, Rochester, N.Y.). Note that these cultures contain tyrosine hydroxylase (TH)⁺ neurons that were exclusively dopaminergic (Traver et al., 2006. Mol Pharmacol. 70(1):30-40). TH⁺ neurons represented approximately 1-2% of the total number of neuronal cells present in these cultures. The evaluation of the survival of DA neurons was performed by counting cells immunopositive for TH as described (Toulorge et al., 2011. Faseb J. 25(8):2563-73).

Culture Systems Used to Model Midbrain Dopaminergic Neuron Cell Death

Spontaneous Dopaminergic (DA) Cell Death Model

We used a model system in which DA cell loss was spontaneous. The demise of DA neurons was progressive after plating to reach approximately 65-70% after 10 DIV (Guerreiro et al., 2008. Mol Pharmacol. 74(4):980-9).

GDNF Withdrawal Model

To evaluate the efficacy of our compounds in a more mature population of DA neurons, we also used a variation of the previous model system. More precisely, spontaneous DA cell death was prevented up to 10 DIV by a chronic treatment with GDNF (20 ng/mL) to favor the maturation of healthy DA neurons. Then, DA cell death was triggered by total withdrawal of GDNF for the next 5 days.

MPP+ Intoxication Model

Treatments with MPP⁺ were performed in cultures where the spontaneous death process was prevented by long-term exposure to depolarizing concentrations of K⁺ (30 mM), in the presence of the glutamate receptor antagonist MK801 (5 μM) to prevent unwanted excitotoxic insults as described previously (Douhou et al., 2001. J Neurochem. 78(1):163-74). Treatments with MPP⁺ and potential neuroprotective molecules were carried out between 5 and 7 DIV. The number of microglial cells was evaluated in this same model since MPP⁺ is known to induce microglial proliferation (Henze et al., 2005. J Neurochem. 95(4):1069-77).

Cortical Cell Cultures

Animals were treated in accordance with the Guide for the Care and Use of Laboratory Animals (National Research Council, 1996), European Directive 86/609, and the guidelines of the local institutional animal care and use committee. Cultures were prepared from the cortex of gestational age 15.5 Wistar rat embryos (Janvier Breeding Center, Le Genest St Isle, France). Dissociated cells in suspension obtained by mechanical trituration of cortical tissue pieces were seeded onto tissue culture supports precoated with 1 mg/mL polyethylenimine diluted in borate buffer pH 8.3 according to the protocol previously described for mesencephalic cultures. The cultures were then maintained in N5 medium supplemented with 5 mM glucose, 5% horse serum, and 0.5% fetal calf serum, except for the first 3 DIV, when the concentration of fetal calf serum was 2.5% to favor initial maturation of the cultures (Guerreiro et al., 2008. Mol Pharmacol. 74(4):980-9). They were fed daily by replacing 70% of the medium. Routinely, cortical cultures were established on Nunc 24-well culture plates (Thermofischer Scientific, Rochester, N.Y.).

Culture System Used to Model Cortical Neuron Cell Death

Excitotoxicity Model

Glutamate (75 μM) was added to cortical cultures between 12 and 14 DIV in the presence or the absence of potential neuroprotective treatments. The cultures were then fixed in formaldehyde and immunostained using anti-MAP-2 antibody. The area occupied by MAP-2⁺ neurons was assessed using ImageJ.

Oxidative Stress Model

H₂O₂ (75 μM) was added to cortical cultures between 12 and 14 DIV in the presence or the absence of potential neuroprotective molecules. The cultures were then fixed in formaldehyde and immunostained using anti-MAP-2 antibody. The area occupied by MAP-2′ neurons was assessed using ImageJ.

Quantification of Neuronal Survival and Microglial Cells Number

The cultures were fixed for 12 min using 4% formaldehyde in Dulbecco's phosphate-buffered saline (PBS), then washed twice with PBS before an incubation step at 4° C. for 24 h with the following antibodies. A monoclonal anti-TH antibody diluted 1/5000 (ImmunoStar, Inc., Hudson, Wis.) or a polyclonal anti-TH antibody diluted 1/1000 (US Biologicals, Salem, Mass.) was used to assess the survival of DA neurons. A monoclonal anti-MAP2 antibody diluted at 1/250 (clone AP20, Sigma-Aldrich) was used to assess the survival of cortical neurons. Microglial cells were characterized using a mouse anti-Ibal antibody (1/50; clone MRC OX-42; Pharmingen, BD Biosciences, Le Pont-de-Claix, France). All antibodies were diluted in PBS containing 0.2% Triton X-100, except the mouse anti-Ibal which was diluted in PBS only. Detection of the primary antibodies was performed with an Alexa Fluor-488 conjugate of an anti-mouse IgG antibody or with an Alexa Fluor-555 conjugate of an anti-rabbit antibody (1:500).

Measurement of Cytosolic Ca²⁺ Levels

Cytoplasmic free calcium levels were measured in individual cortical neurons using Cal-520 (AAT Bioquest). In brief, cultures grown for 7 to 8 days were incubated with 10 μM Cal-520. for 30 min at 37° C., washed twice with serum-free glucose-supplemented N5 medium to remove excess indicator, and then left to recover in the presence of test compounds for 30 min before assessment. The fluorescent signal (excitation, 480 nm; emission, 510 nm) was quantified using the Simple-PCI software from C-Imaging Systems and a Nikon (Tokyo, Japan) TE-300 inverted microscope equipped with an ORCA-ER digital camera from Hamamatsu (Bridgewater, N.J.). The average pixel intensity over the surface of each cell body was determined under the different experimental conditions. Background fluorescence was subtracted from raw data, and the results were expressed as a percentage of mean fluorescence intensity per cell under control conditions. A minimum of 180 cells were analysed under each test condition.

Measurement of Glucose Uptake

Glucose uptake was measured in individual cortical neurons using the fluorescent glucose analogue 2-NBDG (Abcam). In brief, cultures grown for 7 days were incubated with 2-NBDG (300 μM) for 30 min at 37° C. in the presence of test compounds in glucose-free N5 medium, washed twice to remove excess indicator, and then left to recover for 30 min before assessment. The fluorescent signal (excitation, 465 nm; emission, 540 nm) was acquired using an Arrayscan (ThermoFischer Scientific) and quantified using ImageJ. The average pixel intensity over the surface of each cell body was determined under the different experimental conditions. Background fluorescence was subtracted from raw data, and the results were expressed as a percentage of mean fluorescence intensity per cell under control conditions. 100 cells were analysed under each test condition.

Statistical Analysis

Simple comparisons between two groups were performed with Student's t test. Multiple comparisons against a single reference group were made by one-way analysis of variance followed by Dunnett's test when possible. When all pairwise comparisons were required, the Student-Newman-Keuls test was used. S.E.M. values were derived from at least three independent experiments.

In Vivo Experiments

Animals

Animals were treated in accordance with the Guide for the Care and Use of Laboratory Animals (National Research Council, 1996), with the European Directive 2010/63/EU, and the guidelines of the local institutional animal care and use committee. For all studies, 8 to 10 weeks-old male C57Bl/6 mice (Janvier, France) were used. They were maintained on a 12:12 h light/dark cycle with lights on at 8 a.m. The room temperature was kept at 20° C., with free access to standard diet and tap water.

Experimental Design

Three in vivo mouse models were used: the 6-OHDA mouse model, the CBE mouse model and the MPTP mouse model. For 6-OHDA mouse model, the intoxication protocol was based on the unilateral stereotaxic intrastriatal injection of 6-OHDA in the right striatum. Intravenous injections of treatments were made the day before the surgical stereotaxic procedure Animals were sacrificed 8 days after the surgical stereotaxic procedure.

For the CBE mouse model, animal received each day for 9 consecutive days i.p. injection of CBE (100 mg/kg, Toronto Chemical, Canada). Intracerebral or intravenous injections of treatments were made the day before the first injection of CBE Animals were sacrificed one day after the last CBE injection.

For the MPTP mouse model, 4 injections of MPTP (20 mg/kg) were made. Intravenous injection of anti-CD38 clone HB7 antibody were done 2 days before MPTP injection. Mice were sacrificed 7 days after MPTP injections.

Surgical Stereotaxic Procedure

For unilateral 6-OHDA mouse model and intracerebral injection of HB7 antibody in the CBE mouse model, animals were anesthetized using Chloral hydrate (400 mg/kg, i.p.) and placed into a stereotactic frame adapted for mice. The injection was performed using a Hamilton syringe at the following coordinates: AP: +0.85 cm, ML: ±2 cm, DV: −3.4 cm (corresponding to the atlas of Franklin and Paxinos, 1997). A total volume of 2.5 μL containing or not 5 μg of 6-OHDA diluted in PBS in the presence or the absence of test treatments was injected. The needle was left in place for 10 min after the injection before retraction.

Apomorphine-Induced Contralateral Rotations

The unilateral striatal injection of 6-OHDA leads to an ipsilateral lesion of the dopaminergic neurons in the substantia nigra. The degree of damage can be estimated by a behavioral test: the apomorphine-induced rotation test. Apomorphine is an agonist of the dopaminergic receptor D1 and D2 leading to a stereotypical contralateral rotational activity of severely lesioned mice. The more important the lesion, the more important the number of rotations.

The mice were injected i.p. with 1 mg/kg of apomorphine and placed in a transparent Plexiglas tube. The rotations were recorded with cameras during 30 minutes and counted. This test was performed 8 days after the injection of 6-OHDA, just before euthanasia of the animals

Tissue Preparation

Mice were sacrificed by cervical dislocation, and beheaded to collect brain and blood samples. The right and the left striata were dissected on an ice-cold plastic dish, weighed, and transferred into 200 μl 0.2 N perchloric acid The remaining part of the brain was fixed in a paraformaldehyde solution (4%) during 1 day before being soaked in sucrose (30%) during 2 days and then froze at −80° C. for further analysis.

Striatal Dopamine Levels Quantification

Brain tissue pieces immersed in perchloric acid (0.2N) were sonicated for 10 s and the resulting homogenates were centrifuged at 13,000 g for 20 min at 4° C. Supernatants were filtered through a 0.2 μm membrane and filtrates were stored at −80° C. until further analysis. Before injection into high-performance liquid chromatography (HPLC), 100 μL of the samples were mixed with 10 μL phosphate buffer (2 M, pH 7.5) and 5 μL ascorbic acid (0.3 mg/mL). The potential for electrochemical detection was set at +0.65 V and the column temperature was maintained at 19° C. The mobile phase (a buffered aqueous solution containing 15.9% methanol, 1.25 mM octane-1 sulfonic acid sodium salt and 76% of a buffer containing 0.7 M KH₂PO₄, 1 mM EDTA, 31 mM triethylamine, at pH 3) was delivered onto a reversed phase C18 column (250×4.6 mm, bonded silica, Sunfire, Waters, Guyancourt, France).

IL-10 ELISA Assay

Blood samples collected in collection tube (Microvette® 500 EDTA-K3, SARSTEDT) were centrifuged at 3000 rotations per minute during 15 minutes. Supernatants (plasma) were collected and transferred into tubes and froze at −80° C. for further analysis. IL-10 levels in plasma samples were assayed using an ELISA IL-10 kit (BioLegend) according to the indications for use specified by the manufacturer.

Brain Slicing, Immunofluorescence and Neurons Enumeration

Each brain was sliced using a freezing microtome at −30° C. The thickness of each slice was 20 μm. The slicing was done around the striatum (50 slices from slice 21 to slice 30 according to Allen Mouse Brain) and the substantia nigra (80 slices from slice 51 to 63 according to Allen Mouse Brain).

Slices were then washed out in PBS and incubated with either anti-Tyrosine Hydroxylase to stain dopaminergic neurons (Abcam), anti-Iba-1 (Abcam) to stain microglial cells, or anti-GFAP (Abcam) to stain reactive astrocytes, during 2 days, at 4° C., with agitation. Slices were incubated with corresponding secondary antibody in the presence of DAPI (Sigma Aldrich) to stain cell's nuclei during 2 hours at room temperature, and mounted on gelatin-coated slides.

The slices were imaged using a Nikon TE2000 U equipped with a Hamamatsu ORCE-ER camera or with a Zeiss Axio Vert.A1 equipped with an axiocam 503 mono camera. Image analysis was done using Image J software or Zen (Zeiss).

Statistical Analysis

The statistical analyses and figures will be made on the software Sigma Plot. Different tests may be performed depending on the data: One-way ANOVA and post hoc tests, if the conditions of application are respected. If not, Kruskal-Wallis tests will be performed.

1-17. (canceled)

18. A method for preventing and/or treating a neurodegenerative disease and/or an inflammatory disease in a subject in need thereof, said method comprising administering to said subject a compound which specifically binds to CD38 and activates the opening of NAADP receptors Two Pore Channels TPC1 and/or TPC2.

19. The method according to claim 18, wherein said compound is selected from the group consisting of:
an antibody, an antigen-binding fragment thereof or an antigen-binding antibody mimetic; and
a small organic molecule.

20. The method according to claim 18, wherein said compound increases intracellular NAADP levels in neurons and/or immune cells, by inhibiting the NAADP hydrolase activity of CD38 or by activating the NAADP synthase activity of CD38.

21. The method according to claim 18, wherein said compound is an anti-CD38 antibody, an antigen binding fragment thereof or an antigen-binding antibody mimetic, which specifically binds to a peptide comprising amino acids 220 to 285 of SEQ ID NO: 1.

22. The method according to claim 18, wherein said compound is an anti-CD38 antibody, an antigen binding fragment thereof or an antigen-binding antibody mimetic, which specifically binds to cysteine 254 and/or cysteine 275 of SEQ ID NO: 1.

23. The method according to claim 18, wherein said compound is an anti-CD38 antibody or an antigen binding fragment thereof or an antigen-binding antibody mimetic, which specifically binds to the 5^{th} C-terminal disulfide loop involving cysteine 254 and cysteine 275 of SEQ ID NO: 1.

24. The method according to claim 18, wherein said compound induces CD38 internalization.

25. The method according to claim 18, wherein said compound is an anti-CD38 antibody, an antigen binding fragment thereof or an antigen-binding antibody mimetic, which specifically binds to human CD38 with a K_{D} inferior or equal to 10⁻⁷, as may be determined by biosensor analysis.

26. The method according to claim 18, wherein said compound is a humanized monoclonal antibody.

27. The method according to claim 18, wherein said compound is a small organic molecule which inhibits the NAADP hydrolase activity of CD38 with an IC₅₀ inferior or equal to 5 μM or activates the NAADP synthase activity of CD38 with an EC₅₀ inferior or equal to 5 μM.

28. The method according to claim 18, wherein said compound is a small organic molecule, which specifically binds to at least one amino acid of human CD38 with SEQ ID NO: 1 selected from the group comprising glutamic acid 146, aspartic acid 155 and glutamic acid 226.

29. The method according to claim 18, wherein the neurodegenerative disease is selected from the group consisting of Parkinson's disease and related disorders including Parkinson's disease, Parkinson-dementia, autosomal recessive PARK2 and PARK6-linked Parkinsonism, atypical parkinsonian syndromes, including, progressive supranuclear palsy, corticobasal degeneration syndrome, Lewy bodies dementia, multiple system atrophy, Guadeloupean Parkinsonism and Lytigo-bodig disease; motor neuron diseases including amyotrophic lateral sclerosis, frontotemporal dementia, progressive bulbar palsy, pseudobulbar palsy, primary lateral sclerosis, progressive muscular atrophy, spinal muscular atrophy and post-polio syndrome; neuro-inflammatory diseases; Alzheimer's disease and related disorders including early stage of an Alzheimer's disorder, mild stage of an Alzheimer's disorder, moderate stage of an Alzheimer's disorder, mild to moderate stage of an Alzheimer's disorder, advanced stage of an Alzheimer's disorder, mild cognitive impairment, vascular dementia, mixed dementia, Pick's disease, argyrophilic grain disease, posterior cortical atrophy, Wernicke-Korsakoff Syndrome; prion diseases; lysosomal storage diseases; leukodystrophies; Huntington's Disease; multiple sclerosis; Down syndrome; spinal and bulbar muscular atrophy; HIV-Associated Neurocognitive Disorder; Tourette Syndrome; autosomal dominant spinocerebellar ataxia; Friedreich's Ataxia; Dentatorubral pallidoluysian atrophy; myotonic dystrophy; schizophrenia; age associated memory impairment; autism and autism spectrum disorders; attention-deficit hyperactivity disorder; chronic pain; alcohol-induced dementia; progressive non-fluent aphasia; semantic dementia; spastic paraplegia; fibromyalgia; post-Lyme disease; neuropathies; withdrawal symptoms; Alpers' disease; cerebro-oculo-facio-skeletal syndrome; Wilson's disease; Cockayne syndrome; Leigh's disease; neurodegeneration with brain iron accumulation; opsoclonus myoclonus syndrome; alpha-methylacyl-CoA racemase deficiency; Andermann syndrome; Arts syndrome; Marinesco-Sjögren syndrome; mitochondrial membrane protein-associated neurodegeneration; pantothenate kinase-associated neurodegeneration; polycystic lipomembranous osteodysplasia with sclerosing leukoencephalopathy; riboflavin transporter deficiency neuronopathy; and ataxia telangiectasia.

30. The method according to claim 18, wherein the neurodegenerative disease is selected from the group consisting of Parkinson's disease, Lewy body dementia, multiple system atrophy, Alzheimer's disease, progressive supranuclear palsy, corticobasal degeneration syndrome, frontotemporal dementia, amyotrophic lateral sclerosis, spinal and bulbar muscular atrophy, stroke, traumatic brain injuries, Huntington's disease, multiple sclerosis, Friedreich's ataxia, Charcot-Marie-Tooth disease, Creutzfeld-Jacob disease and other prion diseases, leukodistrophies, and lysosomal storage disorders.

31. The method according to claim 18, wherein the inflammatory disease is selected from the group consisting of neuroinflammatory diseases, Gaucher's disease, autoimmune diseases, allergy, asthma, hepatitis, reperfusion injury, type 2 diabetes and transplant rejection.

32. The method according to claim 18, further comprising administering to said subject at least one second therapeutic agent selected from the group consisting of neuroprotective agents, symptomatic agents, probiotics and antibodies used to neutralize aggregated and aggregation-prone proteins, wherein the compound and the at least one second therapeutic agent are formulated for separate, simultaneous, or sequential administration.

33. A method of manufacturing a compound which specifically binds to CD38 and activates the opening of NAADP receptors Two Pore Channels TPC1 and/or TPC2, which comprises the step of selecting a compound which specifically binds to SEQ ID NO: 1 and which activates the opening of NAADP receptors Two Pore Channels TPC1 and/or TPC2.

34. The method according to claim 33, wherein said method further comprises a step of selecting a compound which inhibits the NAADP hydrolase activity of CD38 or which activates the NAADP synthase activity of CD38.
The method according to claim 33, wherein said method further comprises a step of selecting a compound which induces CD38 internalization.
